SEQUENCE LISTING (6 sequences)

SEQ ID NO: 1 - protein, 314 aa, Artificial Sequence
  MAP HAAH - Construct Ia
DRAMAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 2 - protein, 176 aa, Artificial Sequence
  MAP HAAH - Construct II
LDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDP

SEQ ID NO: 3 - protein, 238 aa, Artificial Sequence
  MAP HAAH - Construct III
GTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETRTWEEGKVLIFDDSFEHEVWQDASSFRLIFIVDVWHPELTPQQRRSLPAI

SEQ ID NO: 4 - protein, 324 aa, Artificial Sequence
  GpD-HAAH-1lambda fusion
HMTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIVGGSGPVGPGGSGASSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 5 - protein, 199 aa, Artificial Sequence
  MAP HAAH - Construct I
STSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 6 - protein, 14 aa, Artificial Sequence
  linker
GGSGPVGPGGSGAS

(19) United States
(12) Patent Application Publication
(10) Pub. No.: US 2020/0121773 A1
(43) Pub. Date: Apr. 23, 2020

(54) COMPOSITIONS AND METHODS FOR MANUFACTURING BACTERIOPHAGE CANCER VACCINES AND USES THEREOF

(71) Applicant: SENSEI BIOTHERAPEUTICS, INC., Gaithersburg, MD (US)

(72) Inventors: Solomon S. Stewart, Gaithersburg, MD (US); Samindhi M. Wu, Gaithersburg, MD (US); Steven A. Fuller, Gaithersburg, MD (US); Hossein A. Ghanbari, Potomac, MD (US); IIdiko Csiki, Blue Bell, PA (US)

(21) Appl. No.: 16/657,366

(22) Filed: Oct. 18, 2019

Related U.S. Application Data
(60) Provisional application No. 62/827,485, filed on Apr. 1, 2019.
(60) Provisional application No. 62/757,445, filed on Nov. 8, 2018.
(60) Provisional application No. 62/748,127, filed on Oct. 19, 2018.

(51) Int. Cl. A61K 39/00; C12N 1/20

(52) U.S. Cl. CPC A61K 39/001154; C12N 1/20; A61K 2039/55555; B82Y 5/00; A61K 2039/884; A61K 2039/545; A61K 2039/804

(57) ABSTRACT

Disclosed herein are methods and compositions for manufacturing nanoparticle bacteriophage-based vaccines that are useful for anti-cancer treatments. Also disclosed herein are methods of using bacteriophage-based vaccines expressing aspartyl (asparaginyl) β-hydroxylase for treating cancer.

### CROSS-REFERENCE TO RELATED APPLICATIONS

This application claims priority to U.S. Provisional Patent Application No. 62/827,485, filed on Apr. 1, 2019, U.S. Provisional Patent Application No. 62/757,445, filed on Nov. 8, 2018 and U.S. Provisional Patent Application No. 62/748,127, filed on Oct. 19, 2018. The disclosure of each of these applications is incorporated herein by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SEBI_001_001US_SeqList_ST25.txt, date recorded: Oct. 18, 2019, file size˜11,860 bytes).

### FIELD OF THE DISCLOSURE

The disclosure relates to the fields of human cancer vaccine therapies, nanoparticle vaccines and methods of manufacturing same.

### BACKGROUND

Bacteriophage preparations produced in Gram-negative bacteria are often contaminated with endotoxin (also known as lipopolysaccharide or LPS). There is a need for methods of manufacturing such bacteriophage preparations that have reduced levels of endotoxin contamination and that are safe for therapeutic use. In some cases, such bacteriophage preparations may express a cancer antigen (e.g., human aspartyl (asparaginyl) β-hydroxylase (HAAH), also known as aspartate β-hydroxylase (ASPH)) and may be used in methods for treating cancer.

### SUMMARY

In some embodiments, the disclosure provides a method of purifying and concentrating a bacterial lysate comprising a lambda-phage expressing a cancer antigen or a fragment thereof to produce a nanoparticle vaccine, the method comprising:

i) performing tangential flow filtration (TFF) on the bacterial lysate comprising a lambda-phage expressing a cancer antigen or a fragment thereof to produce a concentrated bacterial lysate;
ii) adding 100% ethanol to the concentrated bacterial lysate to produce a bacterial lysate and ethanol mixture having a 25% ethanol concentration;
iii) performing TFF on the bacterial lysate and ethanol mixture to produce a concentrated ethanol-treated bacterial lysate;
iv) diluting the ethanol-treated bacterial lysate and treating the ethanol-treated bacterial lysate with ultraviolet (UV) light to produce a UV-treated, ethanol-treated bacterial lysate;
v) performing TFF on the UV-treated, ethanol-treated bacterial lysate to produce a nanoparticle vaccine.

In some cases, the TFF in any of the steps described above is performed at a feed flow rate of about 400 mL/minute and a permeate flow rate of about 100 mL/minute. In some cases, the TFF is performed at a Feed pressure (Fp) of about 5.5, a Retentate pressure (Rp) of about 3.5, a Permeate pressure (Pp) of about 2.0 and a Transmembrane pressure (TMP) of about 2.5.

In some embodiments, step ii) of the method described above comprises the steps of (a) adding 200 proof dehydrated alcohol at 42.85 mL per 100 mL of concentrated bacterial lysate to a final concentration of 30% ethanol and stirring the mixture for about 2.5 hours at room temperature; (b) incubating the mixture produced in step (a) overnight at room temperature to allow a precipitate and a clear ethanol-lysate phase to form; (c) separating the clear ethanol-lysate phase from the precipitate; and (d) adjusting the ethanol concentration of the ethanol-lysate phase to 25%.

In some aspects, step ii) of the method described above reduces a level of endotoxin in the concentrated bacterial lysate.

In some embodiments, step iii) of the method described above comprises concentrating the ethanol-treated bacterial lysate to about 50 mL.

In some aspects, step iv) of the method described above comprises using a UV water purifier system with UV monitor to treat the concentrated bacterial lysate and ethanol mixture. In some cases, step iv) of the method described above inactivates lambda-phage in the concentrated bacterial lysate and ethanol mixture.

In some embodiments, a level of endotoxin in the nanoparticle vaccine is below about 10 EU/10¹⁰ particles, below about 1.5 EU/10¹⁰ particles, below about 1.2 EU/10¹⁰ particles or below about 1.0 EU/10¹⁰ particles.

In some cases, the level of endotoxin in the nanoparticle vaccine is reduced about 25%, about 30%, about 35%, about 40%, about 45%, about 50%, about 75%, about 80%, about 90% or about 99% compared to the level of endotoxin in the bacterial lysate.

In some embodiments, the cancer antigen expressed by a lambda-phage used in the methods and compositions described herein is expressed on human cancer cells. In some cases, the cancer antigen is human aspartyl (asparaginyl) β-hydroxylase (HAAH). In some examples, the lambda-phage expresses amino acids 113-311 from the N-terminal region of HAAH fused at the C-terminus of the lambda-phage head decoration protein D (gpD).

The disclosure also provides a nanoparticle vaccine produced by any of the methods described herein.

The disclosure further provides a method for eliciting an antibody response, the method comprising administering to a subject an effective amount of the nanoparticle vaccine described herein. The disclosure also provides a method of treating a symptom of or ameliorating cancer in a subject, the method comprising administering to the subject an effective amount of the nanoparticle vaccine described herein. In some embodiments, the nanoparticle vaccine comprises lambda-phage expressing or comprising a protein comprising the amino acid sequence of SEQ ID NO:4, and the nanoparticle vaccine is administered at a dose from about 2×10¹⁰ particles up to about 3×10¹¹ particles. In some embodiments, the nanoparticle vaccine is administered at a dose of about 2×10¹⁰ particles, about 1×10¹¹ particles or about 3×10¹¹ particles.

In some embodiments, up to 15 cycles of the nanoparticle vaccine are administered, and each cycle comprises a treatment period and a rest period. In some embodiments, the treatment period is about 1 day, and the rest period is about 20 days. In some embodiments, the treatment period is about 1 day, and the rest period is about 41 days. In some embodiments, the treatment period is about 1 day, and the rest period is about 71 days. In some embodiments, four cycles are administered. In some embodiments, six cycles are administered. In some embodiments, the nanoparticle vaccine is administered until the subject exhibits disease progression or toxicity. In some embodiments, the nanoparticle vaccine is administered for up to 24 months if the subject does not exhibit disease progression.

In some embodiments, the subject has prostate, liver, bile duct, brain, breast, colon, lung, head-and-neck, ovarian or pancreatic cancer or a hematological malignancy. In some embodiments, the cancer is an HAAH-expressing cancer. In some examples, the subject has a biochemical recurrence of prostate cancer. In some embodiments, the subject has chronic myelomonocytic leukemia or myelodysplastic syndrome.

### BRIEF DESCRIPTION OF THE DRAWINGS

FIG. 1 is a schematic depicting the SPIRIT platform for generating tumor specific antigen (TSA) immunotherapies.

FIG. 2 is a schematic depicting the development of the SNS-301 (HAAH Nanoparticle Vaccine, HAAH-1λ) immunotherapy from the SPIRIT platform.

FIG. 3 is a table describing the characteristics of human aspartyl (asparaginyl) β-hydroxylase (ASPH), the tumor specific antigen (TSA) targeted by SNS-301. FIG. 3 also shows images of the embryonic expression of ASPH in mice, the immunohistochemical staining of ASPH in prostate tissue and a diagram of the modification of Notch protein by ASPH. ¹ Ince, et al. Cancer Res, (2000); ² de la Monte, et al., J. Hepatol. (2006); ³ Dinchuk, et al. J. Biol. Chem. (2002); ⁴ Patel, et al. Amer. J. Hum. Genetics (2014); ⁵ Data on file; ⁶ Aihara et al, Hepatology (2015); ⁷ Luu et al, Hum Pathol (2009); ⁸ Wang, Hepatology (2010); ⁹ Dinchuk et al. J of Bio Chem (2002); ¹⁰ Gao, Am J Cancer Res (2017).

FIG. 4 is a schematic depicting the Phase 1 study design for SNS-301 in ASPH+ Prostate Cancer Patients with Biochemical Recurrence (BRPC).

FIG. 5 is a table summarizing adverse events (AE) in the Phase 1 study for SNS-301 immunotherapy in ASPH+ Prostate Cancer Patients with Biochemical Recurrence (BRPC).

FIG. 6 is a graph showing that ASPH-specific antibody titers increased as serum ASPH decreased in a representative patient (patient 001-001) after treatment with SNS-301 immunotherapy.

FIG. 7 is a bar graph showing T-cell responses (% CD4⁺ IFNγ) in patient 001-001 after treatment with SNS-301 immunotherapy compared to responses in an unvaccinated subject.

FIG. 8 is a bar graph showing levels of ASPH-specific B-cell levels in representative patients after treatment with SNS-301 immunotherapy.

FIG. 9A is a table showing that SNS-301 immunotherapy leads to increased prostate-specific antigen (PSA) doubling time as PSA velocity is decreased in patients treated with SNS-301 immunotherapy. FIG. 9B depicts two graphs showing the PSA response to SNS-301 immunotherapy in two representative patients.

FIG. 10 depicts a diagram of an SNS-301 bacteriophage vector displaying 300-400 copies of bacteriophage gpD-ASPH fusion protein. FIG. 10 also depicts a diagram of the effects of SNS-301 on various immune system components.

FIG. 11 is a series of plots showing innate immune responses in patients after treatment with SNS-301 immunotherapy. Natural Killer (NK) cells were detected by flow cytometry. PBMCs were stained with antibodies against CD45, CD3, CD16 and CD56. NK cells were CD45⁺, CD3⁻, CD16⁺ and CD56⁺. Dot plots are representative data from single individuals. Scatter plot includes multiple data points per patient all subsequent to multiple treatment cycles.

FIG. 12 is a bar graph showing anti-ASPH antibody (Ab) titers in patients after treatment with SNS-301 immunotherapy. Anti-ASPH antibody levels were measured every 3 weeks at dosing using a tumor cell-based immunoassay. Low and Mid dose cohorts (n=3); High dose cohort (n:==6). The X-axis labels indicate the timing of the measurements in relation to SNS-301 administration, where “C” refers to the cycle, and “D” refers to the day. Thus, “C1D1” refers to “Cycle 1, Day 1”. At each time point, the bars for the cohorts are presented in the following order from left to right: “low dose”, “mid dose” and “high dose”.

FIG. 13 is a bar graph showing ASPH-specific B-cell responses in patients after treatment with SNS-301 immunotherapy. ASPH-specific B-cells were assessed by flow cytometry using fluorescently labeled recombinant ASPH protein. Assessments were from PBMCs collected every 3 weeks at dosing. Low and Mid dose cohorts (n=3); High dose cohort (n=6). The X-axis labels indicate the timing of the measurements in relation to SNS-301 administration, where “C” refers to the cycle, and “D” refers to the day. Thus, “CID1” refers to “Cycle 1, Day 1”. At each time point, the bars for the cohorts are presented in the following order from left to right: “low dose”, “mid dose” and “high dose”.

FIG. 14 is a series of scatter plots and FIG. 15 is a bar graph showing ASPH-specific B-cell responses in a representative patient (patient 003-002) after treatment with SNS-301 immunotherapy. ASPH-specific B-cells from patient 003-002 (mid dose) were assessed by flow cytometry using fluorescently labeled recombinant ASPH protein. B-cells were selected using CD19 coated beads and gated as CD45⁺, CD19⁺, CD20⁺ cells. The labels across the top of FIG. 14 and the X-axis labels in FIG. 15 indicate the timing of the measurements in relation to SNS-301 administration, where “C” refers to the cycle, and “D” refers to the day. Thus, “C1D1” refers to “Cycle 1, Day 1”.

FIG. 16A and FIG. 16B are bar graphs showing ASPH-specific CD4⁺ (FIG. 16A) and CD8⁺ (FIG. 16B) T-cell responses in patients after treatment with SNS-301 immunotherapy. ASPH-specific T-cells were assessed by flow cytometry by ex vivo stimulation of mixed lymphocyte cultures with SNS-301 and rASPH for 1-7 days. IFNγ was trapped on the cell surface and used to isolate activated T-cells which were gated using based on CD4⁺ and CD8⁺ expression, counted by flow cytometry and compared to counts of total CD4⁺ or CD8⁺ T-cells. Low and Mid dose cohorts (n=3); High dose cohort (n=1). The X-axis labels indicate the timing of the measurements in relation to SNS-301 administration, where “C” refers to the cycle, and “D” refers to the day. Thus, “C1D1” refers to “Cycle 1, Day 1”. At each time point, the bars for the cohorts are presented in the following order from left to right: “low dose”, “mid dose” and “high dose”.

FIG. 17 is a series of scatter plots and FIG. 18 is a bar graph showing ASPH-specific T-cell responses in a representative patient (patient 003-002) after treatment with SNS-301 immunotherapy. ASPH-specific CD4⁺ and CD8⁺ T-cells were assessed by flow cytometry by ex vivo stimulation of mixed lymphocyte cultures with SNS-301 and rASPH for 1-7 days. IFNγ was trapped on the cell surface and used to isolate activated T-cells which were subsequently counted by flow cytometry and compared to counts of total CD4⁺ and CD8⁺ T-cells. The right-side labels in FIG. 17 and the X-axis labels in FIG. 18 indicate the timing of the measurements in relation to SNS-301 administration, where “C” refers to the cycle, and “D” refers to the day. Thus, “C2D22” refers to “Cycle 2, Day 22”. In FIG. 18, at each time point, the bars for the T-cell type are presented in the following order from left to right: “CD4⁺” and “CD8⁺”.

FIG. 19 is a line graph showing anti-ASPH antibody titers in three cohorts of patients after treatment with SNS-301 immunotherapy. Anti-ASPH antibody levels were measured every 3 weeks at dosing using a tumor cell-based immunoassay. “Anti-ASPH Lo” refers to patients administered 2×10¹⁰ particles every 21 days for 3 doses (n=3). “Anti-ASPH Mid” refers to patients administered 1×10¹¹ particles every 21 days for 3 doses (n=3). “Anti-ASPH Hi” refers to patients administered 3×10¹¹ particles every 21 days for 3 doses (n=6). Arrows indicate peak antibody titers. The X-axis labels indicate the timing of the measurements in relation to SNS-301 administration, where “C” refers to the cycle, and “D” refers to the day. Thus, “CID1” refers to “Cycle 1, Day 1”.

FIG. 20 is a line graph showing ASPH-specific B-cell responses in three cohorts of patients after treatment with SNS-301 immunotherapy. ASPH-specific B-cells were assessed by flow cytometry using fluorescently labeled recombinant ASPH protein. Assessments were from PBMCs collected every 3 weeks at dosing. Percentage of ASPH-specific B-cells is shown on the y-axis. “B-cell Lo” refers to patients administered 2×10¹⁰ particles every 21 days for 3 doses (n=3). “B-cell Mid” refers to patients administered 1×10¹¹ particles every 21 days for 3 doses (n=3). “B-cell Hi” refers to patients administered 3×10¹¹ particles every 21 days for 3 doses (n=6). Arrows indicate peak B-cell responses. The X-axis labels indicate the timing of the measurements in relation to SNS-301 administration, where “C” refers to the cycle, and “D” refers to the day. Thus, “C1D1” refers to “Cycle 1, Day 1”.

FIG. 21 is a line graph showing anti-phage antibody titers in three cohorts of patients after treatment with SNS-301 immunotherapy. Anti-phage antibody levels were measured every 3 weeks at dosing using a tumor cell-based immunoassay. “Anti-Phage Lo” refers to patients administered 2×10¹⁰ particles every 21 days for 3 doses (n=3). “Anti-Phage Mid” refers to patients administered 1×10¹¹ particles every 21 days for 3 doses (n=3). “Anti-Phage Hi” refers to patients administered 3×10¹¹ particles every 21 days for 3 doses (n=6). The X-axis labels indicate the timing of the measurements in relation to SNS-301 administration, where “C” refers to the cycle, and “D” refers to the day. Thus, “C1D1” refers to “Cycle 1, Day 1”.

FIG. 22 shows the amino acid sequence (SEQ ID NO: 4) of the GpD-HAAH-1 fusion protein. The sequence portions shown in N-terminus to C-terminus order are: (1) GpD sequence, (2) linker sequence; and (3) HAAH sequence.

FIG. 23 shows a timeline of the dosing and administration of SNS-301 in a proposed Phase 2 clinical trial. “C” refers to the cycle, and “Q” stands for “every.”

### DETAILED DESCRIPTION

Disclosed herein are methods for manufacturing bacteriophage-based anti-cancer vaccines that express tumor specific antigens or immunogenic fragments thereof. In some aspects, the methods reduce a level of endotoxin (also known as lipopolysaccharide or LPS) present in the bacterial lysate used to produce the bacteriophage-based vaccine material.

In some embodiments, the bacteriophage used in the methods and compositions disclosed herein is lambda-phage. In some embodiments, the bacterial lysate used in the methods and compositions of the invention is Gram-negative (for example, Escherichia coli) bacterial lysate.

Thus, in some aspects, the disclosure provides a method of purifying and concentrating a bacterial lysate comprising a bacteriophage (e.g., lambda-phage) expressing a cancer antigen or a fragment thereof to produce a nanoparticle vaccine, the method comprising

i) performing tangential flow filtration (TFF) on the bacterial lysate comprising a lambda-phage expressing a cancer antigen or a fragment thereof to produce a concentrated bacterial lysate;
ii) adding 100% ethanol to the concentrated bacterial lysate to produce a bacterial lysate and ethanol mixture having an about 25% ethanol concentration;
iii) performing TFF on the bacterial lysate and ethanol mixture to produce a concentrated ethanol-treated bacterial lysate;
iv) diluting the ethanol-treated bacterial lysate and treating the ethanol-treated bacterial lysate with ultraviolet (UV) light to produce a UV-treated bacterial lysate and ethanol mixture;
v) performing TFF on the UV-treated bacterial lysate and ethanol mixture to produce a nanoparticle vaccine.

In any method steps requiring performing TFF, the TFF may be performed at a feed flow rate of about 400 mL/minute and a permeate flow rate of about 100 mL/minute. Furthermore, in any method steps requiring performing TFF, the TFF may be performed at a Feed pressure (Fp) of about 5.5, a Retentate pressure (Rp) of about 3.5, a Permeate pressure (Pp) of about 2.0 and a Transmembrane pressure (TMP) of about 2.5.

In some aspects, the step of “adding 100% ethanol to the concentrated bacterial lysate to produce a bacterial lysate and ethanol mixture having a 25% ethanol concentration” may itself comprise multiple steps. For example, this step may comprises the steps of (a) adding 200 proof dehydrated alcohol at about 42.85 mL per 100 mL of concentrated bacterial lysate to a final concentration of 30% ethanol and stirring the mixture for about 2.5 hours at room temperature; (b) incubating the mixture produced in step (a) overnight at room temperature to allow a precipitate and a clear ethanol-lysate phase to form; (c) separating the clear ethanol-lysate phase from the precipitate; and (d) adjusting the ethanol concentration of the ethanol-lysate phase to about 25%. In some embodiments, the about 25% ethanol-lysate mixture is filtered through a glass fiber filter before proceeding with subsequent steps of the method.

In some cases, the step of “performing TFF on the bacterial lysate and ethanol mixture to produce a concentrated bacterial lysate and ethanol mixture” (e.g., step iii) comprises concentrating the ethanol-treated bacterial lysate to about 50 mL.

In some cases, the UV light treatment step (e.g., step iv) comprises using a UV water purifier system with UV monitor to treat the ethanol-treated bacterial lysate.

In some cases, the UV light treatment step (e.g., step iv) inactivates lambda-phage in the ethanol-treated bacterial lysate.

Any of the manufacturing or production methods described herein may further comprise a step of inoculating a bacterial stock with a bacteriophage, incubating the infected bacteria for a suitable time and then preparing a bacteriophage-containing bacterial lysate that is used in subsequent purification and concentration steps.

In some cases, the ethanol treatment in the methods described herein reduces a level of endotoxin in the concentrated bacterial lysate. In some embodiments, a level of endotoxin in the nanoparticle vaccine is below about 10 EU/10¹⁰ particles, below about 1.5 EU/10¹⁰ particles, below about 1.2 EU/10¹⁰ particles or below about 1.0 EU/10¹⁰ particles. In some embodiments, the level of endotoxin in the nanoparticle vaccine is reduced about 25%, about 30%, about 35%, about 40%, about 45%, about 50%, about 75%, about 80%, about 90% or about 99% compared to the level of endotoxin in the bacterial lysate.

In some aspects, a bacteriophage used in the methods and compositions disclosed herein expresses a cancer antigen that is expressed on human cancer cells. Such an antigen may also be referred to as a tumor-specific antigen (TSA). In some embodiments, the cancer antigen is human aspartyl (asparaginyl) β-hydroxylase (alternatively abbreviated as HAAH or ASPH). “rASPH” refers to “recombinant ASPH”. In some embodiments, the cancer antigen or a portion of the cancer antigen is fused to the lambda-phage head decoration protein D (gpD).

In some aspects, a bacteriophage (e.g., lambda-phage) comprises a fusion protein comprising a portion of the HAAH protein fused to gpD or a portion of gpD. In some embodiments, a portion of the HAAH protein is fused at the C-terminus of gpD. In some embodiments, the fusion protein comprises a linker sequence between the gpD sequence and the HAAH sequence. In some embodiments, a linker sequence comprises or consists of GGSGPVGPGGSGAS (SEQ ID NO:6). In some embodiments, a bacteriophage comprises a fusion protein comprising a gpD-encoding sequence and an antigenic fragment of at least 9 amino acids, at least 15 amino acids, at least 20 amino acids, at least 25 amino acids, at least 30 amino acids, at least 35 amino acids, at least 40 amino acids, at least 45 amino acids, at least 50 amino acids, at least 75 amino acids or at least 100 amino acids from any one of SEQ ID NO: 1, SEQ ID NO:2, SEQ ID NO:3 and SEQ ID NO:5. In some embodiments, a fusion protein comprising a portion of the HAAH protein fused to gpD does not comprise any sequence from the HAAH amino acid sequence having homology to human Junctin protein or human Humbug protein.

In some embodiments, a bacteriophage (e.g., lambda-phage) expresses an HAAH construct described in U.S. Pat. No. 9,744,223 or U.S. Patent Application Publication No. 2017/0072034 A1. In some embodiments, a lambda-phage expresses one, two, three or four of the HAAH constructs shown in Table 9. In some cases, a bacteriophage (e.g., lambda-phage) expresses amino acids 113-311 from the N-terminal region of HAAH fused at the C-terminus of gpD. SEQ ID NO:5 consists of amino acids 113-311 from the N-terminal region of HAAH. In some embodiments, a bacteriophage (e.g., lambda-phage) comprises a protein comprising or consisting of the amino acid sequence of SEQ ID NO:4. In some embodiments, a bacteriophage (e.g., lambda-phage) comprises a protein comprising or an amino acid sequence at least about 95% identical, at least about 96% identical, at least about 97% identical, at least about 98% identical or at least about 99% identical to the amino acid sequence of SEQ ID NO:4.

In some embodiments, a bacteriophage (e.g., lambda-phage) displays at least about 200, at least about 300 or at least about 400 copies of an extracellular domain of the HAAH protein or a portion of an extracellular domain of the HAAH protein on the bacteriophage's coat.

The disclosure further encompasses the nanoparticle vaccine (e.g., bacteriophage-based vaccine) produced by any of the methods described herein. In some embodiments, the disclosure provides the SNS-301 (HAAH Nanoparticle Vaccine, HAAH-1λ) produced by any of the methods described herein. SNS-301 expresses the HAAH construct I shown in Table 9. In some embodiments, a nanoparticle vaccine produced by or used in any of the methods described herein does not comprise an adjuvant (e.g., does not comprise an exogenous adjuvant). In some embodiments, the nanoparticle vaccine is formulated for intradermal administration.

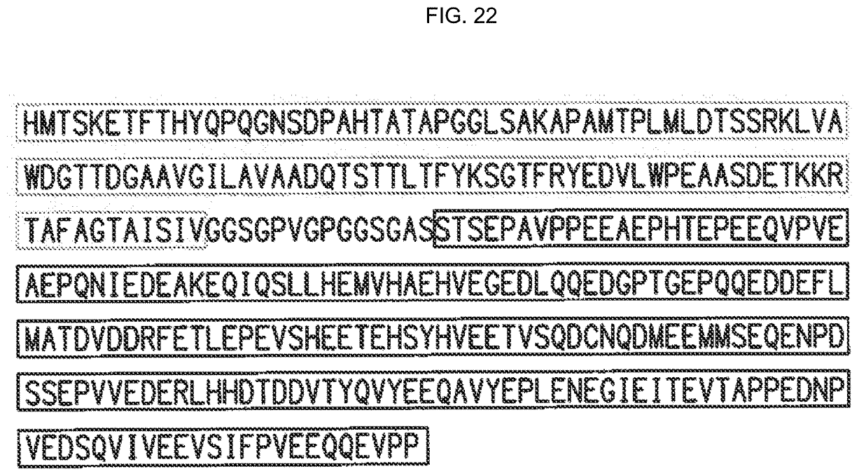

In some embodiments of the methods disclosed herein, the nanoparticle vaccine is SNS-301. SNS-301 is also referred to as HAAH Nanoparticle Vaccine or HAAH-1λ. SNS-301 is composed of lambda-phage that displays portions of the HAAH protein sequence as a fusion protein with the phage gpD head protein. Specifically, the lambda-phage in SNS-301 displays or comprises a protein comprising or consisting of SEQ ID NO:4 (FIG. 22).

In some embodiments, the SNS-301 (HAAH Nanoparticle Vaccine, HAAH-1λ) drug product is formulated in sterile phosphate-buffered saline (10 mM NaPO₄, 0.15 M NaCl), pH 7.4. The vaccine may be filled to a 1 mL volume in a single-use Type 1 glass cartridge sealed with a latex free butyl rubber stopper and a crimp cap with a butyl rubber septum. The vaccine may be delivered intradermally using the 3M hollow microstructured transdermal system (hMTS) device. The drug product may be stored at 2-8° C.

The disclosure also provides a method for eliciting an immune response, the method comprising administering to a subject an effective amount of the nanoparticle vaccine described herein (or produced by the methods described herein). For example, the immune response may be an innate immune response, an antibody response and/or a T-cell response. In some embodiments, the immune response is an increase in the number of natural killer cells in a subject. In some examples, the immune response may be specific for the cancer antigen expressed by the bacteriophage-based vaccine. For example, administration of the nanoparticle vaccine may increase the percentage of cancer antigen-specific (e.g., HAAH-specific) T-cells producing IFNγ (interferon gamma) and/or the percentage of cancer antigen-specific (e.g., HAAH-specific) B-cells. In some embodiments, the cancer antigen-specific B-cells are CD45⁺, CD19⁺ and CD20⁺ cells. In some embodiments, an immune response may be elicited in a subject who has cancer (e.g., an HAAH-expressing cancer).

The disclosure further provides a method of treating a symptom of or ameliorating cancer in a subject, the method comprising administering to the subject an effective amount of the nanoparticle vaccine described herein (or produced by the methods described herein). The disclosure further provides a method of reducing progression of cancer in a subject, the method comprising administering to the subject an effective amount of the nanoparticle vaccine described herein (or produced by the methods described herein).

In any of the methods described herein, the nanoparticle vaccine (e.g., SNS-301) may be administered to a subject at one of the following doses: (1) about 2×10¹⁰ particles; (2) about 1×10¹¹ particles; or (3) about 3×10¹¹ particles. In any of the methods described herein, the nanoparticle vaccine (e.g., SNS-301) may be administered to a subject at one of the following dosage regimens: (1) about 2×10¹⁰ particles every 21 days for 3 doses; (2) about 1×10¹¹ particles every 21 days for 3 doses; or (3) about 3×10¹¹ particles every 21 days for 3 doses. In any of the methods described herein, the nanoparticle vaccine (e.g., SNS-301) may be administered to a subject at one of the following dosage regimens: (1) 2×10¹⁰ particles every 21 days for 3 doses; (2) 1×10¹¹ particles every 21 days for 3 doses; or (3) 3×10¹¹ particles every 21 days for 3 doses.

As defined herein, the term “particles” describes UV-inactivated bacteriophage. In some embodiments, the concentration of the particles and the size of the particles is determined. In some embodiments, the size of the nanoparticles is equivalent to the diameter of the lambda phage head. In some embodiments, the size of the particles is between 48 and 65 nm in diameter. In some embodiments, the lambda phage head exhibits a diameter between 48 and 65 nm. In some embodiments, the number of particles is measured using the Malvern NanoSight NS 300 particle counter. In some embodiments, the Malvern Nanosight NS300 particle counter counts particles which exhibit a diameter between 10 and 300 nm.

In some embodiments, SNS-301 is administered to a subject as a cycle. As defined herein, a cycle comprises a treatment period and a rest period. During the treatment period, one or more drugs is administered. In some embodiments, the one or more drugs are anti-cancer drugs. The rest period is a length of time that the patient does not receive one or more anti-cancer drugs. The rest period may enable the patient to recover from treatment.

In some embodiments, the nanoparticle vaccine (e.g., SNS-301) is administered in a regimen that has a cycle length of 7 days, 14 days, 21 days, 28 days, 35 days, 42 days or more. The regimen may be repeated for any number of cycles to treat cancer, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more.

In some embodiments, the cycle has a rest period. During the rest period, no SNS-301 and/or other therapeutic agent is administered. In some embodiments, no SNS-301 is administered during the rest period, but another therapeutic agent may be administered. In some embodiments, the length of the rest period is about 1 day per cycle, about 2 days per cycle, about 3 days per cycle, about 4 days per cycle, about 5 days per cycle, about 6 days per cycle, about 7 days per cycle, about 8 days per cycle, about 9 days per cycle, about 10 days per cycle, about 11 days per cycle, about 12 days per cycle, about 13 days per cycle, about 14 days per cycle, about 15 days per cycle, about 16 days per cycle, about 17 days per cycle, about 18 days per cycle, about 19 days per cycle, about 20 days per cycle, about 21 days per cycle, or more. In some embodiments, the rest period is one week or two weeks or three weeks or four weeks or five weeks, or six weeks, or seven weeks, or eight weeks, or nine weeks, or ten weeks, or eleven weeks, or twelve weeks, or thirteen weeks, or more. In some embodiments, the rest period is 20 days. In some embodiments, the rest period is 41 days. In some embodiments, the rest period is 71 days.

In some embodiments, the cycle has a treatment period. During the treatment period, SNS-301 and/or one or more therapeutic agents are administered. In some embodiments, the length of the treatment period is about 1 day per cycle, about 2 days per cycle, about 3 days per cycle, about 4 days per cycle, about 5 days per cycle, about 6 days per cycle, about 7 days per cycle, about 8 days per cycle, about 9 days per cycle, about 10 days per cycle, about 11 days per cycle, about 12 days per cycle, about 13 days per cycle, about 14 days per cycle, about 15 days per cycle, about 16 days per cycle, about 17 days per cycle, about 18 days per cycle, about 19 days per cycle, about 20 days per cycle, about 21 days per cycle, or more. In some embodiments, during the treatment period SNS-301 is administered every day. In some embodiments, during the treatment period, SNS-301 is administered every other day. In some embodiments, during the treatment period, SNS-301 is administered every third day. In some embodiments, during the treatment period, SNS-301 is administered every fourth day. In some embodiments, during the treatment period SNS-301 is administered one time per week, two times per week, three times per week, four times per week, five times per week, six times per week, or seven times per week. In some embodiments, during the treatment period, SNS-301 is administered once. In some embodiments, during the treatment period, SNS-301 is administered twice. In some embodiments, during the treatment period, SNS-301 is administered three times. In some embodiments, during the treatment period, SNS-301 is administered four times or more.

In some embodiments, the nanoparticle vaccine (e.g., SNS-301) is administered to a subject in a regimen, wherein a dose of 1×10¹¹ particles is administered every 3 weeks (+3 days) until week 12 (i.e., 4 doses) then every 6 weeks for 6 more doses (until week 45). Thereafter, the nanoparticle vaccine may be administered every 12 weeks until confirmed disease progression or unacceptable toxicity, or up to 24 months in patients without disease progression.

In any of the methods or uses described herein, the subject may be human.

In any of the methods or uses described herein, the vaccine may be administered intradermally.

In some cases, the subject treated by the methods or compositions described herein may have cancer. In some embodiments, the cancer is prostate, lung, head-and-neck, liver, bile duct, brain, breast, colon, ovarian or pancreatic cancer. In some embodiments, the cancer is HAAH-expressing cancer. In some embodiments, the cancer is HAAH-expressing head-and-neck, lung, colon, pancreatic or prostate cancer.

In some embodiments, a subject is screened for HAAH expression (e.g., by a serum-based immunoassay or by immunohistochemical staining of previously resected tissue) and treated by the methods or the compositions described herein if (or when) the subject is positive for HAAH expression. In some embodiments, a subject has measurable HAAH expression in blood or fresh bone marrow aspirate as measured, for example, by flow cytometry.

In some embodiments, the subject has a biochemical recurrence of prostate cancer. In some embodiments, the subject has a biochemical recurrence of prostate cancer with no evidence of metastases.

In some embodiments, the subject treated by the methods or compositions described herein may have a hematological malignancy. A hematological malignancy is a cancer of the blood. In some embodiments, the hematological malignancy is an HAAH-expressing cancer. Non-limiting examples of hematological malignancies include chronic myelomonocytic leukemia (CMML), Non-Hodgkin lymphoma, Hodgkin lymphoma, chronic lymphocytic leukemia, acute myeloid leukemia, acute lymphoblastic leukemia, multiple myeloma, acute myelogenous leukemia, acute nonlymphocytic leukemia, acute myeloblastic leukemia and acute granulocytic leukemia.

In some embodiments, the subject treated by the methods or compositions described herein has chronic myelomonocytic leukemia (CMML). In some embodiments, the subject with CMML has “high risk CMML” that satisfies the World Health Organization (WHO) criteria for CMML-2, characterized by peripheral blasts of 5% to 19%, and 10% to 19% bone marrow blasts and/or presence of Auer rods. In some embodiments, a subject with CMML has been treated with at least one prior anti-CMML therapy (e.g., hydroxyurea, etoposide or a hypomethylating agent (HMA)). In some embodiments, a subject with CMML has relapsed or is refractory/intolerant of HMAs.

In some embodiments, the subject treated by the methods or compositions described herein may have a myelodysplastic syndrome (MDS). MDSs are a group of cancers in which immature blood cells in the bone marrow do not mature into healthy blood cells. In some embodiments, the subject with MDS has anemia, neutropenia, and/or thrombocytopenia. In some embodiments, the subject with MDS has developed acute myelogenous leukemia (AML). In some embodiments, the subject with MDS has “high risk MDS” that satisfies the Revised International Prognostic Scoring System (IPSS-R) criteria for categorization≥Intermediate Risk-3 (IR-3).

In some embodiments, the subject treated by the methods or compositions described herein has lung cancer. In some embodiments, the lung cancer is a small cell lung cancer or a non-small cell lung cancer. Non-limiting examples of non-small cell lung cancers include squamous cell carcinoma, adenocarcinoma, and large cell anaplastic carcinomas. In some embodiments, tests used to diagnose lung cancer include x-rays, sputum cytology, and biopsy.

In some embodiments, the subject treated by the methods or compositions described herein has head-and-neck cancer. Head-and-neck cancer is a term used to describe cancers that develops in the mouth, throat, nose, salivary glands, oral cancers, or cancer that arises in other areas of the head and neck. In some embodiments, the head-and-neck cancer is a squamous cell carcinoma. Head-and-neck cancer is diagnosed using techniques including biopsy, imaging tests, and endoscopy.

The term “about” when immediately preceding a numerical value means ±0% to 10% of the numerical value, ±0% to 10%, ±0% to 9%, ±0% to 8%, ±0% to 7%, ±0% to 6%, ±0% to 5%, ±0% to 4%, ±0% to 3%, ±0% to 2%, ±0% to 1%, ±0% to less than 1%, or any other value or range of values therein. For example, “about 40” means ±0% to 10% of 40 (i.e., from 36 to 44).

### Numbered Embodiments

The following numbered embodiments are also included within the scope of the instant disclosure.

1. A method of purifying and concentrating a bacterial lysate comprising a lambda-phage expressing a cancer antigen or a fragment thereof to produce a nanoparticle vaccine, the method comprising:

i) performing tangential flow filtration (TFF) on the bacterial lysate comprising a lambda-phage expressing a cancer antigen or a fragment thereof to produce a concentrated bacterial lysate;
ii) adding 100% ethanol to the concentrated bacterial lysate to produce a bacterial lysate and ethanol mixture having an about 25% ethanol concentration;
iii) performing TFF on the bacterial lysate and ethanol mixture to produce a concentrated ethanol-treated-bacterial lysate;
iv) diluting the ethanol-treated bacterial lysate and treating the ethanol-treated bacterial lysate with ultraviolet (UV) light to produce a UV-treated, ethanol-treated bacterial lysate;
v) performing TFF on the UV-treated, ethanol-treated bacterial lysate to produce a nanoparticle vaccine.

2. The method of embodiment 1, wherein the TFF is performed at a feed flow rate of about 400 mL/minute and a permeate flow rate of about 100 mL/minute.

3. The method of embodiment 1 or 2, wherein the TFF is performed at a Feed pressure (Fp) of about 5.5, a Retentate pressure (Rp) of about 3.5, a Permeate pressure (Pp) of about 2.0 and a Transmembrane pressure (TMP) of about 2.5.

4. The method of any one of embodiments 1-3, wherein step ii) comprises the steps of

(a) adding 200 proof dehydrated alcohol at 42.85 mL per 100 mL of concentrated bacterial lysate to a final concentration of 30% ethanol and stirring the mixture for about 2.5 hours at room temperature;
(b) incubating the mixture produced in step (a) overnight at room temperature to allow a precipitate and a clear ethanol-lysate phase to form;
(c) separating the clear ethanol-lysate phase from the precipitate; and
(d) adjusting the ethanol concentration of the ethanol-lysate phase to 25%.

5. The method of any one of embodiments 1-4, wherein step ii) reduces a level of endotoxin in the concentrated bacterial lysate.

6. The method of any one of embodiments 1-5, wherein step iii) comprises concentrating the ethanol-treated bacterial lysate to about 50 mL.

7. The method of any one of embodiments 1-6, wherein step iv) comprises using a UV water purifier system with UV monitor to treat the ethanol-treated bacterial lysate.

8. The method of any one of embodiments 1-7, wherein step iv) inactivates lambda-phage in the ethanol-treated bacterial lysate.

9. The method of any one of embodiments 1-8, wherein a level of endotoxin in the nanoparticle vaccine is below about 10 EU/10¹⁰ particles, below about 1.5 EU/10¹⁰ particles, below about 1.2 EU/10¹⁰ particles or below about 1.0 EU/10¹⁰ particles.

10. The method of any one of embodiments 1-9, wherein the level of endotoxin in the nanoparticle vaccine is reduced about 25%, about 30%, about 35%, about 40%, about 45%, about 50%, about 75%, about 80%, about 90% or about 99% compared to the level of endotoxin in the bacterial lysate.

11. The method of any one of embodiments 1-10, wherein the cancer antigen is expressed on human cancer cells.

12. The method of any one of embodiments 1-11, wherein the cancer antigen is human aspartyl (asparaginyl) β-hydroxylase (HAAH).

13. The method of any one of embodiments 1-11, wherein the lambda-phage expresses amino acids 113-311 from the N-terminal region of HAAH fused at the C-terminus of the lambda-phage head decoration protein D (gpD).

14. The method of any one of embodiments 1-11, wherein the lambda-phage expresses or comprises a protein comprising the amino acid sequence of SEQ ID NO:5 fused at the C-terminus of the lambda-phage head decoration protein D (gpD).

15. The method of any one of embodiments 1-11, wherein the lambda-phage expresses or comprises a protein comprising the amino acid sequence of SEQ ID NO:4.

16. The nanoparticle vaccine produced by the method of any one of embodiments 1-15.

17. A method for eliciting an antibody response in a subject, the method comprising administering to the subject an effective amount of the nanoparticle vaccine of embodiment 16.

18. The method of embodiment 17, wherein the subject has head-and-neck, lung, prostate, liver, bile duct, brain, breast, colon, ovarian or pancreatic cancer or a hematological malignancy.

19. A method for treating a symptom of or ameliorating cancer in a subject, the method comprising administering to the subject an effective amount of the nanoparticle vaccine of embodiment 16.

20. The method of embodiment 17, wherein the cancer is head-and-neck, lung, prostate, liver, bile duct, brain, breast, colon, ovarian or pancreatic cancer or a hematological malignancy.

21. The method of embodiment 18 or 20, wherein the subject has a biochemical recurrence of prostate cancer.

22. The method of embodiment 18 or 20, wherein the hematological malignancy is chronic myelomonocytic leukemia or myelodysplastic syndrome.

23. The method of any one of embodiments 18-22, wherein the cancer is HAAH-expressing cancer.

24. The method of any one of embodiments 17-23, wherein the nanoparticle vaccine is administered at a dose from about 2×10¹⁰ particles up to about 3×10¹¹ particles.

25. The method of any one of embodiments 17-24, wherein the nanoparticle vaccine is administered at a dose of about 1×10¹¹ particles.

26. The method of any one of embodiments 17-25, wherein up to 15 cycles of the nanoparticle vaccine are administered, and wherein each cycle comprises a treatment period and a rest period.

27. The method of embodiment 26, wherein the treatment period is about 1 day, and the rest period is about 20 days.

28. The method of embodiment 26, wherein the treatment period is about 1 day, and the rest period is about 41 days.

29. The method of embodiment 26, wherein the treatment period is about 1 day, and the rest period is about 71 days.

30. The method of any one of embodiments 26-29, wherein four cycles are administered.

31. The method of any one of embodiments 26-29, wherein six cycles are administered.

32. The method of embodiment 25, wherein a dose of about 1×10¹¹ particles is administered every 3 weeks until week 12; and then a dose of about 1×10¹¹ particles is administered every 6 weeks until week 45.

33. The method of any one of embodiments 26-32, wherein the nanoparticle vaccine is administered until the subject exhibits disease progression or toxicity.

34. The method of embodiment 29, wherein the nanoparticle vaccine is administered for up to 24 months if the subject does not exhibit disease progression.

35. A method for eliciting an antibody response in a subject, the method comprising administering to the subject an effective amount of a nanoparticle vaccine comprising lambda-phage expressing or comprising a protein comprising the amino acid sequence of SEQ ID NO:4, wherein the nanoparticle vaccine is administered at a dose from about 2×10¹⁰ particles up to about 3×10¹¹ particles.

36. The method of embodiment 35, wherein the subject has head-and-neck, lung, prostate, liver, bile duct, brain, breast, colon, ovarian or pancreatic cancer or a hematological malignancy.

37. A method for treating a symptom of or ameliorating cancer in a subject, the method comprising administering to the subject an effective amount of a nanoparticle vaccine comprising lambda-phage expressing or comprising a protein comprising the amino acid sequence of SEQ ID NO:4, wherein the nanoparticle vaccine is administered at a dose from about 2×10¹⁰ particles up to about 3×10¹¹ particles.

38. The method of embodiment 37, wherein the cancer is head-and-neck, lung, prostate, liver, bile duct, brain, breast, colon, ovarian or pancreatic cancer or a hematological malignancy.

39. The method of embodiment 36 or 38, wherein the subject has a biochemical recurrence of prostate cancer.

40. The method of embodiment 36 or 38, wherein the hematological malignancy is chronic myelomonocytic leukemia or myelodysplastic syndrome.

41. The method of any one of embodiments 36-40, wherein the cancer is HAAH-expressing cancer.

42. The method of any one of embodiments 35-41, wherein the nanoparticle vaccine is administered at a dose of about 2×10¹⁰ particles, about 1×10¹¹ particles or about 3×10¹¹ particles.

43. The method of any one of embodiments 35-42, wherein up to 15 cycles of the nanoparticle vaccine are administered, and wherein each cycle comprises a treatment period and a rest period.

44. The method of embodiment 43, wherein the treatment period is about 1 day, and the rest period is about 20 days.

45. The method of embodiment 43, wherein the treatment period is about 1 day, and the rest period is about 41 days.

46. The method of embodiment 43, wherein the treatment period is about 1 day, and the rest period is about 71 days.

47. The method of any one of embodiments 35-46, wherein four cycles are administered.

48. The method of any one of embodiments 35-46, wherein six cycles are administered.

49. The method of embodiment 42, wherein a dose of about 1×10¹¹ particles is administered every 3 weeks until week 12; and then a dose of about 1×10¹¹ particles is administered every 6 weeks until week 45.

50. The method of any one of embodiments 35-49, wherein the nanoparticle vaccine is administered until the subject exhibits disease progression or toxicity.

51. The method of embodiment 46, wherein the nanoparticle vaccine is administered for up to 24 months if the subject does not exhibit disease progression.

52. A nanoparticle vaccine comprising lambda-phage expressing or comprising a protein comprising the amino acid sequence of SEQ ID NO:4, wherein the nanoparticle vaccine comprises a dose from about 2×10¹⁰ particles up to about 3×10¹¹ particles, for use in the treatment of cancer.

53. The nanoparticle vaccine for use according to embodiment 52, wherein the cancer is head-and-neck, lung, prostate, liver, bile duct, brain, breast, colon, ovarian or pancreatic cancer or a hematological malignancy.

54. The nanoparticle vaccine for use according to embodiment 53, wherein the subject has a biochemical recurrence of prostate cancer.

55. The nanoparticle vaccine for use according to embodiment 53, wherein the hematological malignancy is chronic myelomonocytic leukemia or myelodysplastic syndrome.

56. The nanoparticle vaccine for use according to any one of embodiments 52-55, wherein the cancer is HAAH-expressing cancer.

57. The nanoparticle vaccine for use according to any one of embodiments 52-56, wherein the nanoparticle vaccine comprises a dose of about 2×10¹⁰ particles, about 1×10¹¹ particles or about 3×10¹¹ particles.

58. The nanoparticle vaccine for use according to any one of embodiments 52-57, wherein up to 15 cycles of the nanoparticle vaccine are administered, and wherein each cycle comprises a treatment period and a rest period.

59. The nanoparticle vaccine for use according to embodiment 58, wherein the treatment period is about 1 day, and the rest period is about 20 days.

60. The nanoparticle vaccine for use according to embodiment 58, wherein the treatment period is about 1 day, and the rest period is about 41 days.

61. The nanoparticle vaccine for use according to embodiment 58, wherein the treatment period is about 1 day, and the rest period is about 71 days.

62. The nanoparticle vaccine for use according to any one of embodiments 52-61, wherein four cycles are administered.

63. The nanoparticle vaccine for use according to any one of embodiments 52-61, wherein six cycles are administered.

64. The nanoparticle vaccine for use according to embodiment 57, wherein a dose of about 1×10¹¹ particles is administered every 3 weeks until week 12; and then a dose of about 1×10¹¹ particles is administered every 6 weeks until week 45.

65. The nanoparticle vaccine for use according to any one of embodiments 52-64, wherein the nanoparticle vaccine is administered until the subject exhibits disease progression or toxicity.

66. The nanoparticle vaccine for use according to embodiment 61, wherein the nanoparticle vaccine is administered for up to 24 months if the subject does not exhibit disease progression.

### Examples

### Example 1: Production of Lambda-Phage Based Cancer Vaccine Targeting Human Aspartyl (Asparaginyl) β-Hydroxylase

A therapeutic cancer vaccine based on and targeting the tumor marker human aspartyl (asparaginyl) β-hydroxylase (abbreviated as HAAH or ASPH) was produced. A portion of the HAAH protein sequence (˜25 kDa in size) was presented on the surface of bacteriophage lambda as a fusion protein with the phage head decoration protein D (gpD). HAAH-1λ contains 199 amino acids (amino acids 113-311; SEQ ID NO:5) from the N-terminal region of HAAH fused at the C-terminus of the gpD head protein. The entire fusion protein has the amino acid sequence of SEQ ID NO:4. The design of the HAAH-1λ construct is described in U.S. Pat. No. 9,744,223 and U.S. Patent Application Publication No. 2017/0072034 A1. The recombinant bacteriophage carry 200-300 copies of the gpD protein on their heads and thus display many copies of the HAAH fragment on their surface.

A HAAH-1λ phage lysate was produced as follows. First, an inoculum was prepared. Six liters of LB-broth with 10 mM MgSO₄ were prepared by using a 10 mL pipette to add 10 mL of MgSO₄ to each of six 1 L bottles of Luria-Bertani medium. Using a 10 mL pipette, 10 mL of LB-broth with 10 mM MgSO₄ were transferred to each of six 50 mL centrifuge tubes. Each centrifuge tube was inoculated with a loop scraping of E. coli W3110 sup-bacterial stock. Each aliquot was mixed gently with a 10 ml pipette. The tubes were transferred to a 37° C. incubator equipped with shaker set at 200 rpm, and incubated overnight.

The inoculum was then used to prepare a phage lysate. 1.3 L of LB-broth with 10 mM MgSO₄ were transferred to each of four 4 L autoclaved glass Erlenmeyer flasks. The contents of the six 50 mL centrifuge tubes were resuspended using a 10 mL pipette. The contents were then pooled by transferring to a 250 mL media bottle. Using a 25 mL pipette, each 4 L flask was inoculated with 13 mL of the pooled overnight culture. The inoculated flasks were incubated at 37° C. in a shaker incubator set at 200 rpm.

One mL samples of the cultures were obtained using a 1 mL pipette every 10 minutes starting at 70 minutes of incubation. OD₆₀₀ was measured. Incubation was continued until the OD reached 0.12±0.02. Each flask culture was infected with HAAH-1λ Working Stock at a MOI of 0.05±0.01 (for example, each flask was infected with 0.9 ml of 5.94×10⁹ pfu/mL of phage) using a 1 mL pipette. Infected flasks were incubated at 37° C. in a shaker incubator set at 250 rpm for 2.5 hours. Using a pipettor, approximately 20 U/mL of Benzonase (for example, 100 μL of 250 U/μL of Benzonase) was added to each flask. Incubation was continued for another 2 hours.

The culture medium was transferred to 500 mL centrifuge bottles. These bottles were centrifuged at 8000 rpm (approximately 11,000×g) at 2-8° C. for 10 minutes in a Sorvall centrifuge using a GS-3 rotor. The supernatant was collected into an autoclaved 4 L Erlenmeyer flask. Filtration in the next step was conducted as each set of bottles from a centrifugation was available. Using bottle top filters, the supernatant was serially filtered through a 0.45μ CA membrane and a 0.22μ PES membrane into a 5 L Corning 1395 bottle. A second HAAH-1λ phage lysate was produced by an identical method. The two lysates were pooled, labeled as “HAAH-1λ lysate” and stored at 2-8° C.

Tangential flow filtration (TFF) was performed on the HAAH-1λ lysate.

Set Up the TFF System:

One Pellicon 88 cm² & 0.11M² Cassette Holder containing 4 Pellicon 2 Ultracel 300 KDa Mini Cassettes was prepared. Two Masterflex pumps and Masterflex tubing were connected to the Pellicon Cassette Holder with inlet tubing and outlet tubing to form a fluid path as follows:
The inlet tubing from the sample reservoir connects through pump 1 to the feed port and the outlet tubing connects to the retentate port and flows back to the sample reservoir.
The filtrate tubing from the permeate port connects through one of a dual rotor of pump 2 into the filtrate reservoir.
The tubing from the dialysis buffer connects through the second rotor of pump 2 into the sample reservoir.

All processes were performed using the following pressure and flow rate specifications:

Feed pressure (Fp)˜5.5, Retentate pressure (Rp)˜3.5, Permeate pressure (Pp)˜2.0, Transmembrane pressure (TMP)˜2.5

Feed flow rate=400 mL/minute, Permeate flow rate=100 mL/minute

Cleaning the TFF System:

The TFF system was flushed with 2 L of deionized water with all ports open, then drained. The system was flushed with 2 L of 0.5 M NaOH with all ports open, followed by recirculation of 1 L of 0.5 M NaOH for 30 minutes, then drained. 1 L of 0.1 M NaOH was recirculated through the system for approximately 5 minutes. The system was shut down with the filters stored in 0.1 M NaOH at room temperature.

Concentration and Diafiltration of HAAH-1λ Lysate:

The 5.0-10.5 L HAAH-1λ lysate was retrieved from 2-8° C. storage and allowed to sit at room temperature for 16-18 hours. The TFF system was retrieved from storage and set up as described in the previous section. The 0.1 M NaOH was drained from the TFF system and flushed with 2 L of Water for Injection (WFI), then drained. One liter of WFI was recirculated through the system for at least 5-10 minutes, then drained. One liter of phosphate-buffered saline (PBS) was recirculated through the system while calibrating to the operational concentration/diafiltration pressure and flow settings listed above. The tubing from the feed and recirculate ports was placed into the vessel containing the HAAH-1λ lysate, which was being mixed slowly on a magnetic stirrer. The tubing from the permeate port was placed into a 10 L vessel to collect the filtrate. The lysate was concentrated to approximately 500 mL. The concentrated lysate was transferred to a 1 L DURAN® bottle. The 10 L vessel was rinsed with approximately 500 mL PBS and added to the concentrated lysate in the DURAN® bottle.

The feed tubing was placed into the 1 L DURAN® bottle and concentrated to approximately 300 mL with slow mixing of the concentrate. The permeate port was closed, and the holdup volume was pumped from the TFF system into the lysate concentrate bottle. Two sequential 5 minute recirculate washes of the TFF system were performed using approximately 200 mL PBS per wash and pooled with the lysate in the 1 L DURAN® bottle. The concentrated lysate was transferred into a 1 L glass graduated cylinder and the volume was adjusted to 1 L with PBS. The concentrated lysate was transferred into a 2 L DURAN® bottle. The 1 L bottle and the 1 L cylinder were rinsed with 100 mL PBS to recover residual lysate and added to the 1 L volume in the 2 L bottle. The TFF system was cleaned as described above.

Ethanol Treatment of HAAH-1λ Concentrated Lysate:

The concentrated lysate 2 L DURAN® bottle was placed on a magnetic stirrer and mixed at moderate speed. 200 proof dehydrated alcohol (100% ethanol) was added slowly at 42.85 mL per 100 mL of concentrated lysate (final concentration of ethanol is 30%). The ethanol-lysate mixture was stirred at 2.5 hours at room temperature. The mixture was divided equally into two 1 L DURAN® bottles and incubated overnight (16-24 hours) at room temperature to allow precipitate to form. The clear upper ethanol-lysate phase was transferred carefully from each 1 L bottle into a 2 L DURAN® bottle. The ethanol concentration was adjusted to 25% by adding PBS at 20 mL per 100 mL. The 25% ethanol-lysate mixture was filtered through a 0.22μ 1 L PES filter unit equipped with a glass fiber prefilter into a 2 L DURAN® bottle.

Concentration and diafiltration of 25% ethanol-lysate: The 0.1 M NaOH was drained from the TFF system and flushed with 2 L of WFI, then drained. One liter of WFI was recirculated through the system for at least 5-10 minutes, then drained. One liter of PBS+25% ethanol was recirculated through the system while calibrating to the operational concentration/diafiltration pressure and flow settings listed above. The 25% ethanol-lysate was concentrated to approximately 200 mL, then transferred to a 250 mL DURAN® bottle and the concentration was continued to approximately 90 mL. The concentrated 25% ethanol-lysate was diafiltered with 2 L of PBS+25% ethanol, followed by diafiltration with 2 L PBS.

The ethanol-treated lysate was concentrated to approximately 50 mL, and the holdup volume was drained into the 250 mL bottle. A 1 mL aliquot was collected for analysis. The permeate port was closed, and 5 L of PBS was recirculated in a 5 L DURAN® bottle through the TFF system for 5-10 minutes. The holdup volume was drained into the bottle with the 5 L PBS recirculate wash. The concentrated/diafiltered ethanol-treated lysate was added to the 5 L DURAN® bottle and stirred slowly to mix.

Ultraviolet (UV) Inactivation of HAAH-1λ Ethanol-Treated Lysate:

A MIGHTY PURE® UV water purifier system with UV monitor (Atlantic Ultraviolet Corporation, Hauppauge, N.Y.) was set up. The drain port was closed. The feed, outlet and drain tubing was placed into a 5 L DURAN® bottle containing 4 L of WFI. A peristaltic pump was used to fill the UV system chamber through the feed port with the WFI until the water drains back into the container through the outlet port. The drain port was opened, and the 4 L of WFI was recirculated through the system for at least 5 minutes. The system was drained. The UV lamp was turned on. The peristaltic pump was used to fill the unit with PBS to just overflowing and allowed to recirculate while the lamp warmed up (15-30 minutes). The UV monitor was adjusted to detect 100% UV intensity in PBS with the lower trip setting adjusted to 90%. The inlet tubing was placed from the UV unit into the container with the 5 L of HAAH-1λ ethanol-treated lysate. The outlet tubing and drain tubing was placed into a 10 L collection bottle. The drain port was closed. The peristaltic pump was used to pump the HAAH-1λ ethanol-treated lysate through the inlet port into the PBS-filled unit at approximately 1 L per minute (515 rpm). The outflow was collected into the 10 L collection bottle. 100% UV detected is desirable during the entire run for complete inactivation of the HAAH-1λ. When the HAAH-1λ ethanol-treated lysate has been completely pumped into the UV unit, the feed tubing was immediately transferred to a bottle containing 3 L of PBS and continued to pump through the UV unit to flush residual HAAH-1λ ethanol-treated lysate into the 10 L collection bottle. The drain port was opened, and the outlet port was closed. The remaining liquid from the UV unit was pumped into the 10 L collection bottle. A 1 mL aliquot was collected for the plaque assay for residual bacteriophage. The UV light was turned off. Five liters of 0.5 M NaOH was recirculated through the UV unit for 10-20 minutes. The NaOH solution was drained and discarded. Five liters of deionized water was recirculated through the UV unit for 5 minutes, then drained. The UV unit was flushed with at least 5 L of deionized water, drained and secured for storage.

Preparation of TFF System for Concentration/Diafiltration of Post-UV-Treated HAAH-1λ Nanoparticles:

The TFF system was cleaned as described above. Then, the TFF system was flushed with 2 L of WFI with all ports open and drained. One liter of WFI was recirculated through the TFF system for 5-10 minutes, then drained. One liter of PBS was recirculated through the TFF system while calibrating to the operational concentration/diafiltration pressure and flow settings listed above.

TFF Concentration of HAAH-1λ Nanoparticles:

Using the TFF unit as prepared in the section above, the HAAH-1λ nanoparticles were concentrated to approximately 500 mL, then transferred to a 500 mL DURAN® bottle. The nanoparticles were concentrated further to approximately 50 mL. The permeate port was closed, and the retentate was recirculated for 5 minutes. The holdup volume was pumped into the retentate bottle. 115 mL of PBS was recirculated through the TFF system for 5 minutes. This solution was collected into a separate bottle as a recirculate wash. The volume of the retentate and recirculate wash materials was measured as they were transferred to the filter units. The retentate and the recirculate wash were filtered separately through 0.22μ PES filter units into sterile 250 mL bottles. The retentate was labeled as “HAAH-1λ Bulk Drug Substance”. A 2 mL aliquot was removed for QC (quality control) testing.

Results of analysis of the HAAH-1λ Bulk Drug Substance are shown in Table 1. The testing was conducted in compliance with cGMP.

TABLE 1
Results of analysis of the HAAH-1λ, Bulk Drug Substance

| Test | Test Method | Specification | Result | |
|---|---|---|---|---|
| Appearance | SOP ANL002 | Clear, Colorless Liquid | Clear, Colorless Liquid | |
| pH | SOP ANL003 | 7.0-7.7 | 7.2 | |
| Identity by Dot Blot | SOP ANL004 | Reactive with HAAH- | Reactive | |
| | | 1 [symbol] -specific antibody | | |
| Endotoxin | SOP ANL005 | Report Result | 1.1 | EU/10¹⁰ Particles |
| Host Cell Protein | SOP ANL006 | Report Result | 141.2 | ng/mg Protein |
| Residual Viable | SOP ANL007 | <100 pfu/10¹⁰ | 0.1 | pfu/10¹⁰ Particles |
| Bacteriophage | | Particles | | |
| Quantitation of | SOP ANL008 | >5 × 10¹¹ | 4.32 × 10¹² | |
| Particle | | Particles/mL | Particles/mL | |
| Concentration by | | | | |
| Particle Analysis | | | | |
| Determination of | SOP ANL008 | Report Result | 55 | nm |
| Median Particle Size | | | | |
| by Particle Analysis | | | | |
| Protein | SOP ANL009 | Report Result | 0.6 | μg/10¹⁰ Particles |
| Determination | | | | |
| Potency by Antigen | SOP ANL015 | Report Result | 5.8 | ng equivalents/ |

| | | | |
|---|---|---|---|
| ELISA | | | 10¹⁰ Particles |
| Western Blot | SOP ANL012 | Main band at ~50 kDa | Main band at ~50 kDa |
| SDS-PAGE | SOP ANL011 | Main band at ~35 kDa, | Main band at ~35 |
| | | secondary band | kDa, secondary band |
| | | at ~60 kDa | at ~60 kDa |
| Bioburden | USP <61> | Total Aerobic Microbial | TAMC: ≤1 CFU/mL |
| | | Count | TYMC: ≤1 CFU/mL |
| | | (TAMC): ≤10 CFU/mL, | |
| | | Total Combined Yeast | |
| | | and Molds Count | |
| | | (TYMC): ≤10 CFU/mL | |

### Example 2: Reduction of Endotoxin in Bacteriophage Lambda Vaccine Manufacturing Process

### Background

The reduction of bacterial endotoxins in bacteriophage produced from E. coli fermentation is important for drug safety. The U.S. Food and Drug Administration has set an upper limit of 5 EU (endotoxin units) per kg body weight for drugs administered parenterally. In previous analyses of bacteriophage lambda preparations, endotoxin co-purified with the bacteriophage and could not be removed by tangential flow filtration alone. The levels of endotoxin present in the vaccine preparations limited the potential human dose and did not provide a sufficient safety margin. Further process development work was required to reduce the endotoxin.

### Process Development for Reduction of Endotoxin

Several chemical treatment methods were evaluated to dissociate the endotoxin from the bacteriophage or to destroy the endotoxin in the centrifuged, diafiltered bacterial lysate that contained the bacteriophage. Each sample was treated initially for 2 hours, then any additional processing, i.e., neutralization, dilution or phase separation, was done, followed by overnight incubation at room temperature. The following day, samples were centrifuged to eliminate any precipitates and the supernatants were tested for bacteriophage and endotoxin. These chemical treatments and the rationale for their use are listed below in Table 2 and the endotoxin measurements are presented in Table 3.

TABLE 2
Chemical Treatment of Processed Lysate for Reduction of Bacterial Endotoxins

| Chemical Treatment | Rationale for Treatment | Treatment Result |
|---|---|---|
| 2M sodium chloride (NaCl) | High salt concentration can | Less than one log |
| Room temperature | dissociate biological materials | reduction in endotoxin |
| incubation, 2 h | from each other | |
| Sodium hydroxide (NaOH) | NaOH can dissociate precipitate | Less than one log |
| Room temperature | or chemically destroy biological | reduction in endotoxin at |
| incubation, 2 h with | materials | 0.05M NaOH and |
| 0.05M and 0.1M | | undesirable precipitation |
| NaOH, then neutralize pH | | of materials at 0.1M |
| with 1M HC1 | | NaOH |
| 5 mg/mL delipidated human | Delipidated HSA is known to | Observed too much |
| serum albumin (HSA) | bind to lipids, possibly would | precipitation, loss of |
| 37° C. incubation for 2 h, | dissociate endotoxin from the | bacteriophage |
| then dilute with phosphate- | bacteriophage | |
| buffered saline, pH 7.2 | | |
| Octanol extraction | Octanol is known to extract | Less than one log |
| Room temperature | lipid-containing materials | reduction in endotoxin |
| incubation, 2 h with | (such as bacterial endotoxins) | |
| octanol in 2:3 ratio to | from aqueous solutions | |
| lysate, allow phases to | | |
| separate, collect aqueous | | |
| phase | | |
| Ethanol | Ethanol can both dissociate and | Increase in endotoxin |
| Room temperature | precipitate biological materials | reduction from 10-30% |
| incubation, 2 h | | ethanol, heavy precipitate |
| with 10-50% ethanol | | and loss of bacteriophage |
| | | at 40-50% ethanol |

TABLE 3
Endotoxin Data for Chemical Treatment of Processed Lysate

| | Trial 1 | Trial 2 | Bacteriophage Loss and |
|---|---|---|---|
| | Endotoxin | Endotoxin | Net Gain in Endotoxin |
| Chemical Treatment | (EU/mL) | (EU/mL) | Removal |
| None-Starting Lysate | 2.6 × 10⁵ | 1.2 × 10⁵ | — |
| 2M NaCl | 7.1 × 10⁴ | ND | Loss of both endotoxin and |
| | | | phage, no net gain |
| 0.05M NaOH | 8.4 × 10⁴ | ND | Loss of both endotoxin and |
| | | | phage, no net gain |
| 0.1M NaOH | Precipitate | ND | No net gain due to |
| | | | precipitation |
| 5 mg/mL delipidated | Precipitate | ND | No net gain due to |
| HSA | | | precipitation |
| Octanol extraction | ND | 6.7 × 10⁴ | Loss of both endotoxin and |
| | | | phage, no net gain |
| 10% Ethanol | 6.0 × 10⁴ | 4.4 × 10⁴ | Loss of both endotoxin and |
| | | | phage, no net gain |
| 20% Ethanol | 4.0 × 10³ | 8.4 × 10² | Approx. 2 log gain in |
| | | | endotoxin, <1 log loss of |
| | | | phage, net gain of 1-1.5 log |
| | | | endotoxin removal |
| 30% Ethanol | 1.5 × 10² | <0.6 × 10² | Approx. 3 log gain in |
| | | | endotoxin, approx.. 1 log |
| | | | loss of phage, net gain of 2 |
| | | | log endotoxin removal |
| 40%, 50% Ethanol | Precipitate | ND | No net gain due to |
| | | | precipitation |

Only ethanol treatment was effective in reducing endotoxin relative to recovery of bacteriophage. An increasingly effective removal was observed with levels of ethanol up to 30%. The two trials presented in Table 3 gave consistent results in endotoxin removal. Subsequent manufacturing of bacteriophage vaccine incorporated a 30% ethanol treatment step, followed by filtration to remove precipitates and diafiltration. This step was inserted prior to ultraviolet irradiation and final diafiltration.

Comparison of Endotoxin in Manufactured Batches of Bacteriophage Vaccine with and without the Ethanol Treatment Step

Endotoxin levels in bacteriophage vaccine batches were compared for materials manufactured with and without the 30% ethanol treatment step. The data for 5 batches prepared without ethanol treatment and 9 batches prepared with ethanol treatment are presented in Table 4. There is a clear reduction in endotoxin of approximately 2 logs for the batches made with the ethanol treatment step. This reduced level of endotoxin now provides for effective dosing of the vaccine and also a good safety margin.

TABLE 4
Endotoxin Levels in Bacteriophage Lambda Vaccine
Lots Manufactured With or Without Ethanol Treatment

| | Mean | |
|---|---|---|
| | Endotoxin ± | Range |
| | Std. Dev. | Endotoxin |
| | (EU/10¹⁰ | (EU/10¹⁰ |
| Batch Description | Particles) | Particles) |
| Batches Manufactured Without | 257 ± 117 | 89-398 |
| Ethanol Treatment (N = 5) | | |
| Batches Manufactured With Ethanol | 4.8 ± 3.6 | 0.5-9.3 |
| Treatment (N = 9) | | |

### Example 3: Phase 1 Clinical Trial of Cancer Vaccine Targeting Human Aspartyl (Asparaginyl) β-Hydroxylase in Men with Biochemically Relapsed Prostate Cancer

A phase 1 clinical trial of cancer vaccine (SNS-301) targeting human aspartyl (asparaginyl) 3-hydroxylase (alternatively abbreviated as HAAH or ASPH) in men with biochemically relapsed prostate cancer was carried out. One third of patients experience a biochemical recurrence (BCR) after radical prostatectomy or radiation therapy (RT). These patients are relatively healthy, immunocompetent and not yet recommended for androgen deprivation therapy. After a decline in PSA test usage, there has been an increased burden of late-stage disease, while the decline in prostate cancer mortality has leveled off. In 2010, GS 7 disease became the most prevalent presentation of prostate tumors at diagnosis at 40% and increasing slightly to 41% in 2014.

There is no FDA-approved immunotherapy for patients who have not progressed to metastatic castration resistant prostate cancer status. The SNS-301 trial was designed for patients with BCR after prostatectomy±RT with no evidence of metastases. It was hypothesized that SNS-301 targeting ASPH overexpressed in prostate cancer would break self-tolerance and lead to anti-tumor immunity. The antigen-specific T-cell mediated response could confer anti-tumor effect as well as demonstrate an increase in the percent of antigen-specific T cells producing IFNγ which serve as a correlate for clinical improvement. SNS-301 delivered using 3M ID device is designed to enhance immunologic responses, thus leading to stabilization of disease progression in BCR prostate cancer patients.

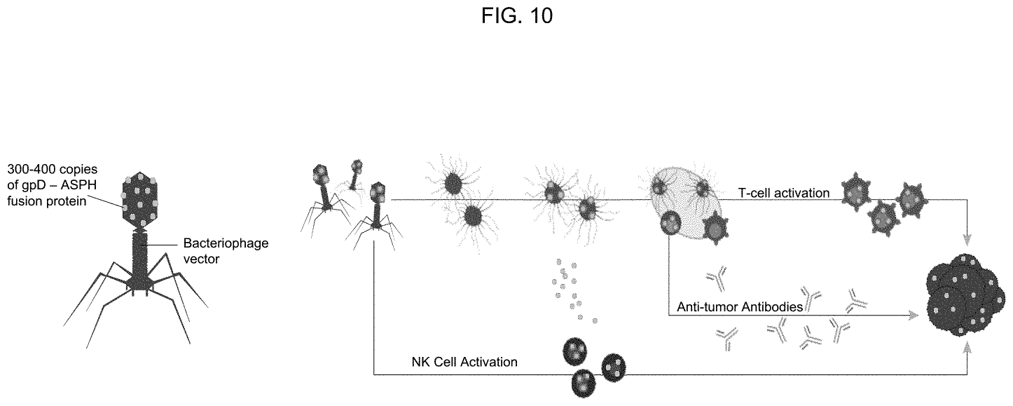

SNS-301 (HAAH Nanoparticle Vaccine, HAAH-1λ) is a T cell immunotherapy targeting human aspartyl (asparaginyl) β-hydroxylase (abbreviated as HAAH or ASPH). SNS-301 is an ASPH-targeted vaccine in which the antigen is integrated onto the coat of a bacteriophage. SNS-301 is an engineered, inactivated bacteriophage expressing a fusion protein of native bacteriophage gpD protein and a selected domain of ASPH (FIG. 10). The fusion protein has the amino acid sequence of SEQ ID NO:4. Bacteriophage is innately immunogenic and requires no exogenous adjuvant. SNS-301 displays a high density of each ASPH fusion product on its surface; 2-3 times more compared to alternate vector systems. SNS-301 targets immune cells to activate ASPH-specific B- and T-cell responses. The vaccine is delivered intradermally to maximize access to sentinel dendritic (Langerhans) cells located in the skin.

SNS-301 contains HAAH-1λ Bulk Drug Substance as produced by the methods described in Example 1. The SNS-301 product is the drug substance filled in a single-use glass cartridge with rubber stopper and crimp cap that is delivered using an intradermal injection device produced by 3M Company, Drug Delivery Systems Division.

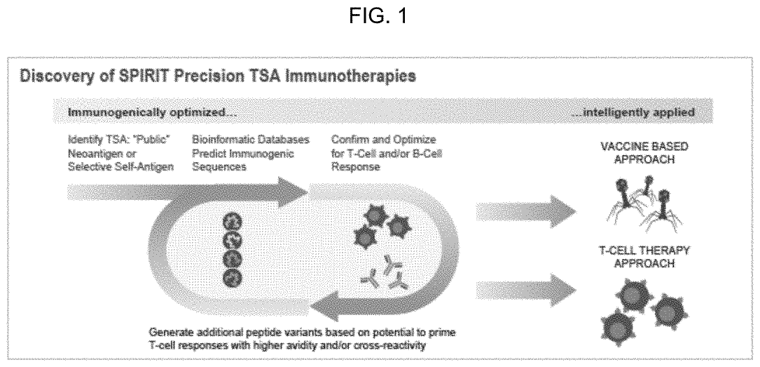

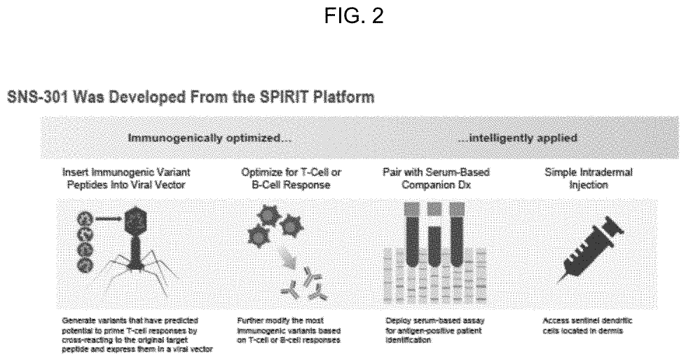

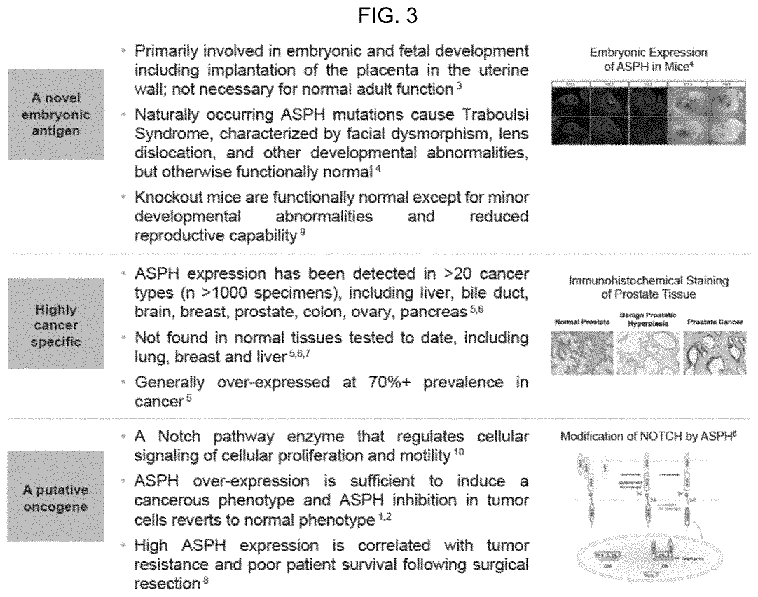

SNS-301 was developed using the SPIRIT platform for generating tumor specific antigen (TSA) immunotherapies (FIG. 1 and FIG. 2). The key features of SNS-301 are shown in Table 5. SNS-301 targets ASPH, an emerging tumor specific antigen (FIG. 3).

TABLE 5
SNS-301 key features

| | |
|---|---|
| Activates | Immediately and potently activates an |
| | innate and adaptive immune response |
| Well-Tolerated | Shown to be well-tolerated and safe in patients |
| Engineerable | Engineered to produce best-in-class |
| | 200-300 copies per particle |
| Conveniently | Delivered intradermally quickly and easily |
| Delivered | using a 3M® micro-needle system |
| Sustainable | Allows for sustained enhancement of immunity |
| | and repeat administrations; >100 doses |
| | administered in cancer patients |
| Readily | Can be manufactured through a simple and |
| Manufacturable | cost-effective process with high yields |

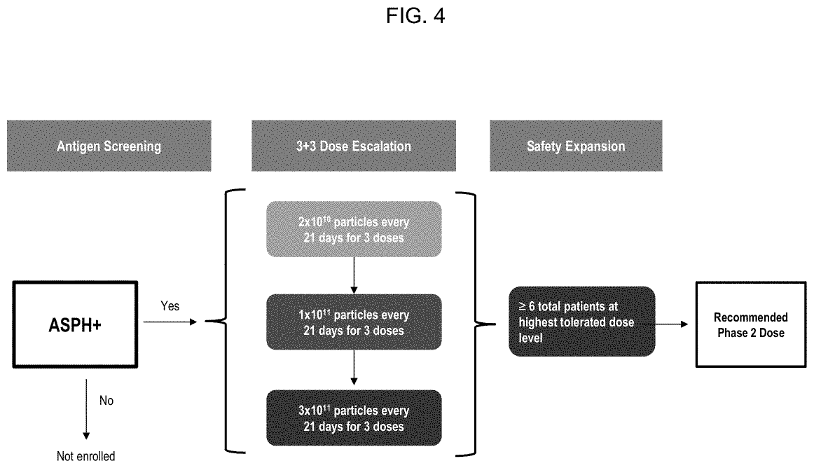

The clinical trial study design is shown in FIG. 4. The objective of the trial was to determine the maximum tolerated dose (MTD) and overall safety of SNS-301 in ASPH+ Prostate Cancer Patients with Biochemical Recurrence (BRPC). 19 subjects were screened at three urological practices. Subjects were screened using a serum-based sandwich ELISA employing anti-ASPH monoclonal antibodies. 17/19 patients screened tested positive for serum ASPH antigen (≥2 ng/ml). (5 subjects either failed other inclusion criteria or decided not to enroll in the study.) 12 subjects were enrolled and treated. These subjects received 6-18 doses of SNS-301 (median=10 doses). The following doses of SNS-301 were administered to subjects: (low dose) 2×10¹⁰ particles every 21 days for 3 doses; (mid dose) 1×10¹¹ particles every 21 days for 3 doses; or (high dose) 3×10¹¹ particles every 21 days for 3 doses.

Patients were vaccinated via intradermal injection every 21 days and whole blood and serum samples were obtained at each visit prior to vaccination. While only required to complete 3 doses, all patients opted to continue on study for at least 6 cycles.

Only 6 adverse events (AEs) considered by the physician to be study related were identified (3 in the same patient) and all were less than or equal to grade 3. The adverse event summary is described in more detail below.

All patients remained progression free while on study (>7 months). All patients experienced dose-dependent ASPH-specific immune responses including B-cell, T-cell and antibody responses.

The primary endpoints of the Phase 1 study were safety and tolerability, recommended phase 2 dose (RP2D). The secondary endpoints were: (1) Immunogenicity as measured by ASPH-specific B-cell and T-cell responses and ASPH-specific antibody production; (2) Activation of the innate immune system as measured by NK cell count; (3) Efficacy as measured by changes in PSA velocity and doubling times.

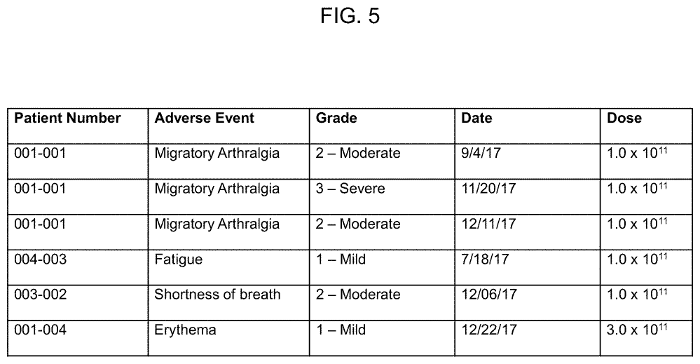

The Phase 1 adverse event (AE) safety summary is shown in FIG. 5. N=6 AEs were considered by physician to be related to study drug, all less than or equal to grade 3 (N=39 AEs total). N=0 severe adverse events (SAEs) were considered to be related to study drug (N=1 SAE total). Safety on all three dosing cohorts was established and recommended phase 2 dose (RP2D) was identified. No dose limiting toxicity was observed. No grade 4-5 AEs were noted. One grade 3 AE of migratory arthralgia (possibly related) was observed as patient was diagnosed with rheumatoid arthritis, and immunization may have contributed to the pain flare.

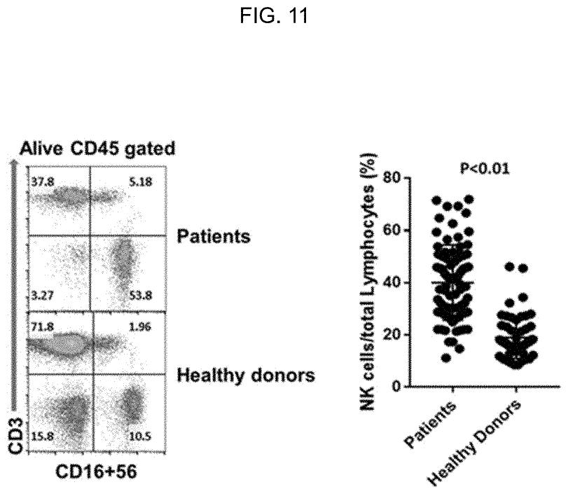

Natural Killer (NK) levels in patients treated with SNS-301 were higher than NK cell levels in healthy donors, indicating activation of the innate immune system by the phage vaccine (FIG. 11). NK cells were detected by flow cytometry. PBMCs were stained with antibodies against CD45, CD3, CD16 and CD56. NK cells were CD45⁺, CD3, CD16⁺ and CD56⁺.

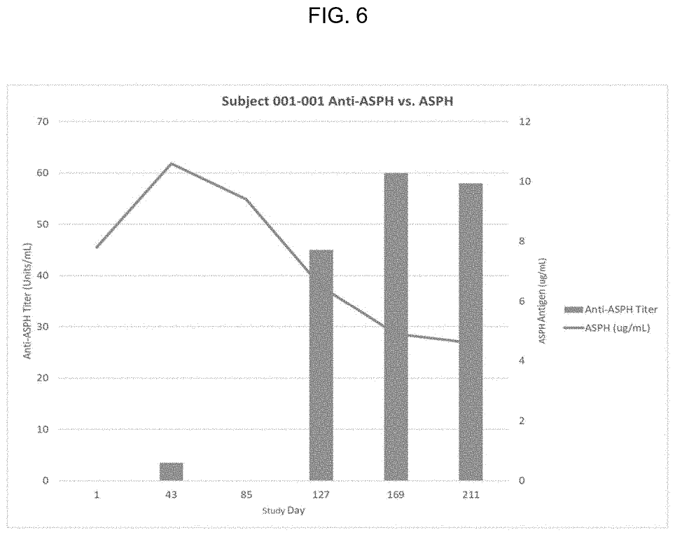

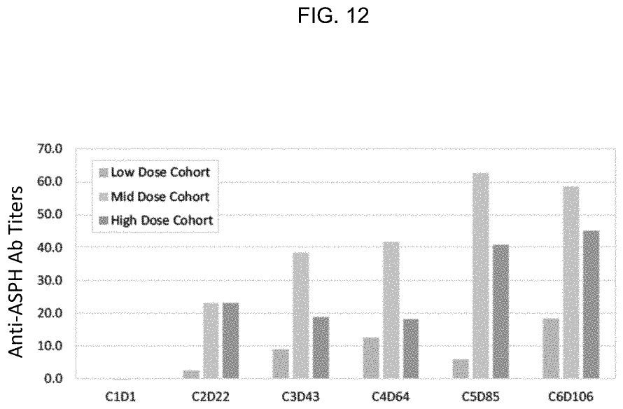

ASPH-specific antibody titers increased as serum ASPH decreased in treated subjects (FIG. 12). Anti-ASPH antibody titers in patient sera were determined with an ELISA method using H460 as the plate-coating antigen. The H460 cell line expresses ASPH and provides a measure of antibodies that have reactivity with native ASPH. Mean anti-ASPH antibody titers for the 3 dose cohorts through the first six patient visits (study day 0-106), were calculated to allow a direct comparison of dose response between dose cohorts (evaluable samples: n=3 for low and mid dose cohorts, n=6 high dose cohort). Results from a representative example (patient 001-001) are shown in FIG. 6.

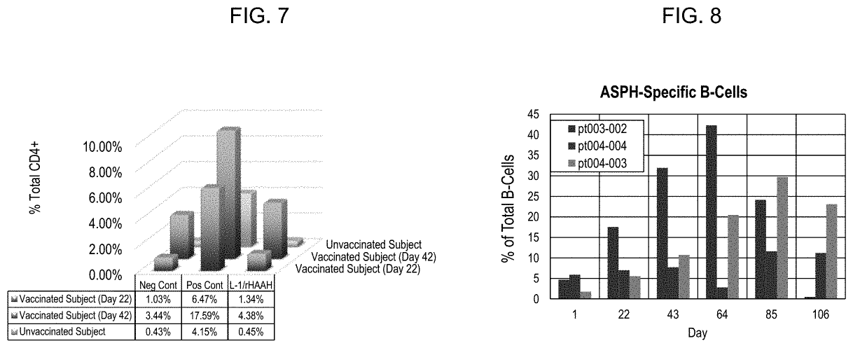

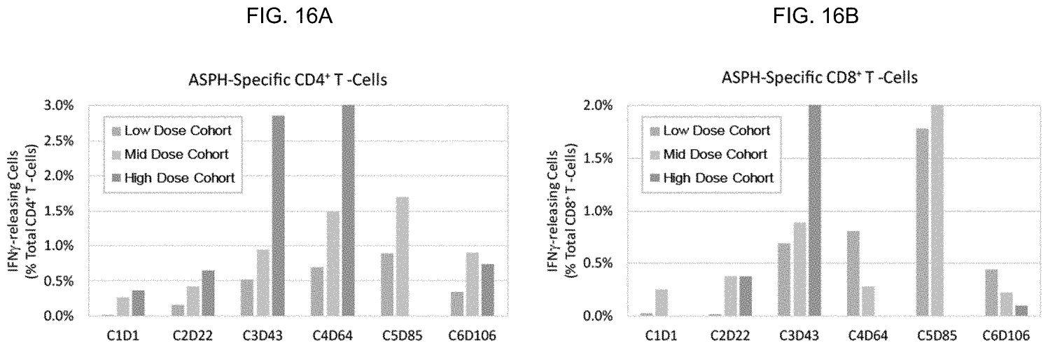

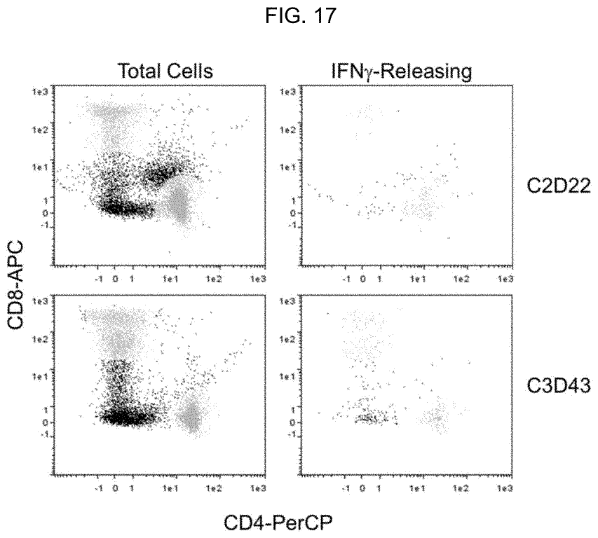

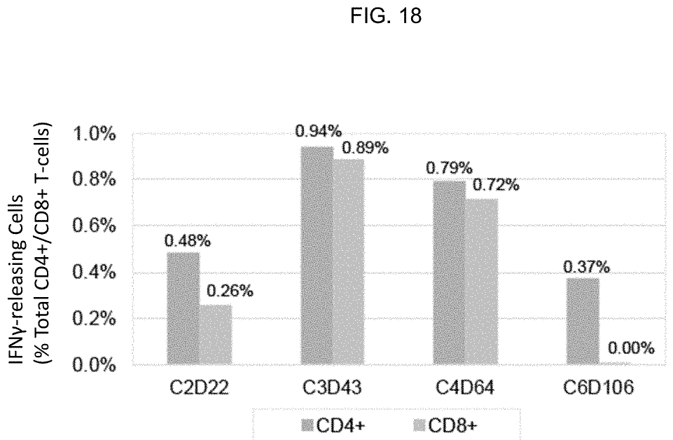

SNS-301 immunotherapy led to ASPH-specific T-cell responses (FIG. 16A and FIG. 16B). ASPH-specific T-cell responses were determined by flow cytometry. Isolated PBMCs were incubated in a mixed lymphocyte culture in the presence of SNS-301 and a recombinant form of ASPH (rASPH) for 16 hours (overnight) to 7 days. When incubated for multiple days, the SNS-301 and rASPH were refreshed in the culture every 3 days. Controls included cells incubated in the absence of any added in vitro stimulation or with a general stimulator of T-cell activation. On the day T-cell numbers were determined, cells were loaded with IFN-γ catch reagent, a bispecific antibody which binds to a T-cell surface marker and to IFN-γ. Cells were incubated for a further 45 minutes to “catch” released IFN-γ on the cellular surface. Cells were subsequently stained with fluorescently labeled antibodies against CD4, CD8 and IFN-γ. The anti-IFN-γ antibody was labeled with FITC. IFN-γ producing T-cells were selected using anti-FITC coated magnetic beads. An unselected portion of the sample was used to determine the total numbers of CD4⁺ and CD8⁺ T-cells and a selected portion of the sample was used to count the numbers of IFN-γ producing CD4⁺ and CD8⁺ T-cells. The percentage of activated, IFN-γ producing, CD4⁺ and CD8⁺ T-cells out of total CD4⁺ and CD8⁺ T-cells were calculated and shown as the average percentage of ASPH-specific T-cells at each treatment cycle per dosage group (evaluable samples: n=3 for low and mid dose cohorts, n=1 high dose cohort). Increased percentage of CD4⁺ T-cells was noted in patients treated with SNS-301 as compared to cells from a control unvaccinated individual, thus demonstrating SNS-301 vaccine/ASPH-specific T-cell stimulation. Similar results were observed across the patients analyzed, with those from a representative patient (patient 001-001) shown in FIG. 7. Results from patient 003-002 are shown in FIG. 17 and FIG. 18. Increases in activated, IFN-γ releasing T-cells were demonstrated. Both ASPH-stimulated CD4⁺ helper T cells and CD8⁺ cytotoxic T-cells showed dose dependent activation over the first 6 cycles of vaccine delivery.

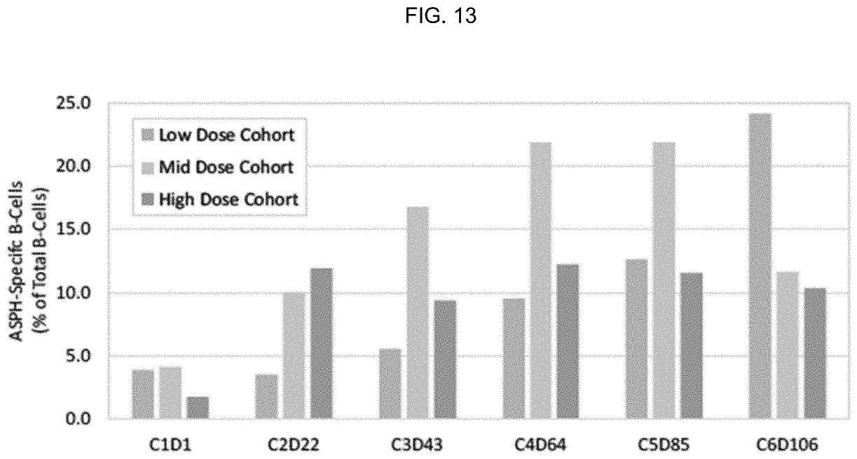

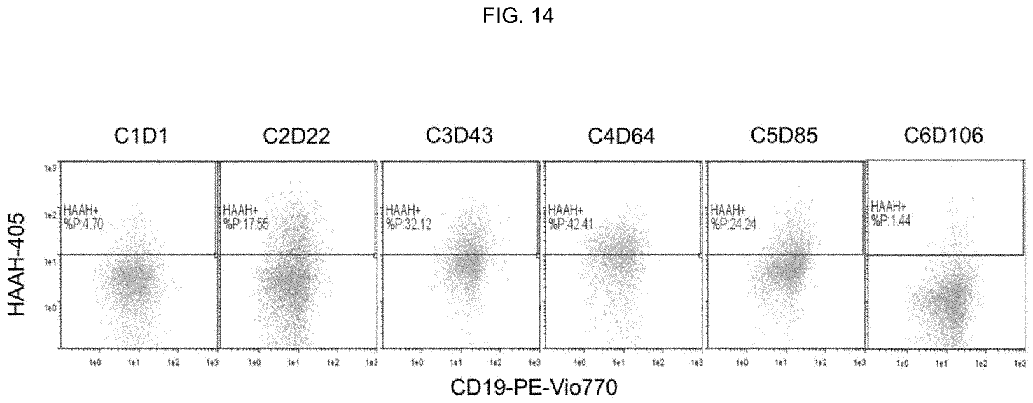

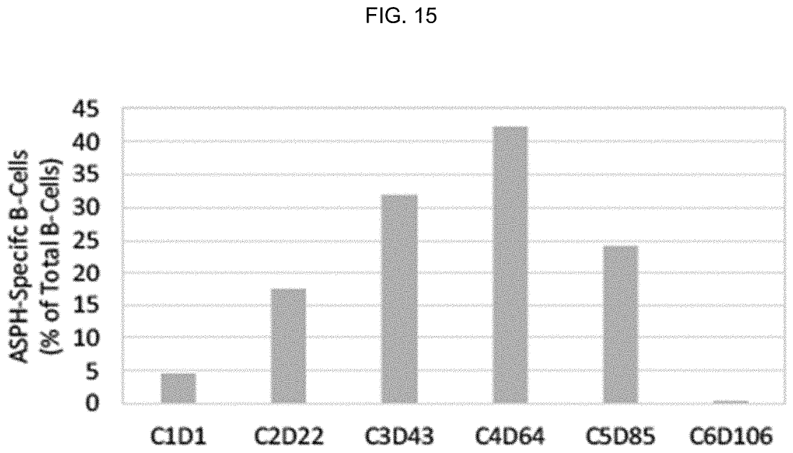

SNS-301 immunotherapy also led to robust ASPH-specific B-cell responses (FIG. 13). ASPH-specific B-cells were quantified by flow cytometry. Fresh PBMCs were isolated from patient blood using a Ficoll-Paque density gradient and cells were stained with fluorescently labeled antibodies against CD45, CD19, and CD20, a fluorescently-labeled recombinant form of ASPH and co-incubated with magnetic beads coated with anti-CD19. Total B-cells were selected by an in-line magnetic column prior to analysis by flow cytometry. Anti-CD45, CD19 and CD20 were used for gating and detection of total B-cells and B-cells that were also stained with recombinant ASPH were counted. The % of ASPH-specific B-cells were calculated and reported as mean % ASPH-specific B-cells at each treatment cycle per dosage group (evaluable samples: n=3 for low and mid dose cohorts, n=6 high dose cohort). ASPH-specific B-cell responses in patients to mid dose are shown in FIG. 8. Results from patient 003-002 are shown in FIG. 14 and FIG. 15. B-cell responses increased over time in patients, reaching a peak and then tapering off over the time period analyzed.

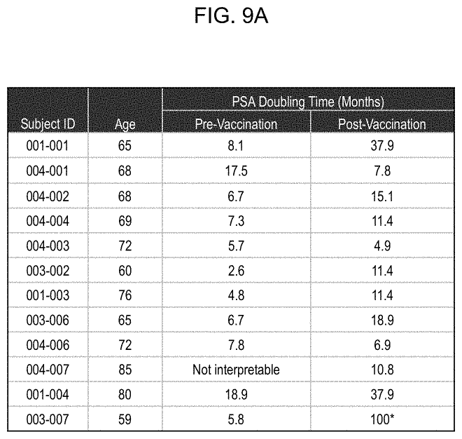

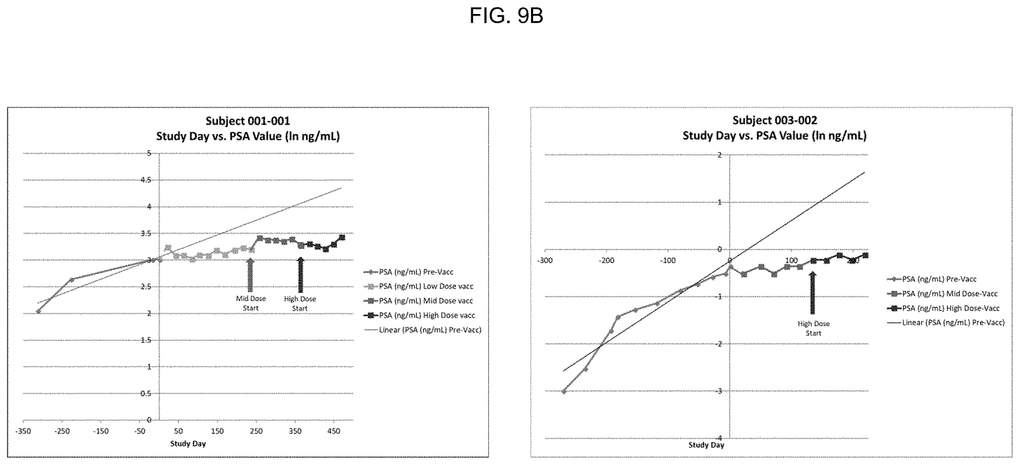

SNS-301 immunotherapy led to increased PSA (Prostate-specific Antigen) doubling time as PSA velocity was decreased (FIG. 9A). Baseline/pre-vaccination PSA doubling time (PSADT) values were calculated using available pre-treatment PSA values, post-baseline/post-vaccination values were calculated using Day 22 through Cycle 6 PSA data (Table 6). Patients with a negative PSA velocity (PSAV) (i.e., slope), PSADT cannot be calculated and these values are denoted as Negative. Improvements in PSADT and PSAV are denoted in bold font, while non-improvements are in non-bold font. PSA response to SNS-301 immunotherapy is shown in two representative patients (patient 001-001 and patient 003-002) (FIG. 9B).

TABLE 6
Effects of SNS-301 immunotherapy on PSA values

| | | | PSA Doubling Time (PSADT, months)/PSA Velocity (PSAV) | | | |
|---|---|---|---|---|---|---|
| Patient | | | Pre-Vaccination | | Post-Vaccination | |
| Number | Age | Dose | PSADT | PSAV | PSADT | PSAV |
| 001-001 | 65 | 2 × 10¹⁰ | 13.8 | 0.0017 | Negative | −0.0017 |
| 004-001 | 68 | 2 × 10¹⁰ | 18.2 | 0.0012 | 6.1 | 0.0038 |
| 004-002 | 68 | 2 × 10¹⁰ | 6.7 | 0.0034 | Negative | −0.000016 |
| 004-004 | 69 | 1 × 10¹¹ | 7.4 | 0.003 | Negative | −0.0017 |
| 004-003 | 72 | 1 × 10¹¹ | 5.6 | 0.004 | 6.0 | 0.0038 |
| 003-002 | 60 | 1 × 10¹¹ | 3.5 | 0.0065 | 16.1 | 0.0014 |
| 001-003 | 76 | 3 × 10¹¹ | 17.4 | 0.001 | 11.0 | 0.002 |
| 003-006 | 65 | 3 × 10¹¹ | 6.9 | 0.003 | 16.6 | 0.0014 |
| 004-006 | 72 | 3 × 10¹¹ | 7.9 | 0.0029 | 6.6 | 0.0034 |
| 004-007 | 85 | 3 × 10¹¹ | 64.0 | 0.00035 | 12.3 | 0.0018 |
| 001-004 | 80 | 3 × 10¹¹ | 24.1 | 0.0009 | 34.2 | 0.0006 |
| 003-007 | 59 | 3 × 10¹¹ | 5.9 | 0.0038 | Negative | −0.0017 |

In conclusion, dose dependent immunogenicity was observed across B-cell and T-cell parameters. PSA doubling time improved for 8 of 11 patients. Overall immune responses occurred faster and were more robust at the two higher doses vs. the lower dose. Immunologic efficacy generally correlated with biochemical responses in these patients. Results from representative patients are shown in Tables 7 and 8. Two mid-dose patient individual values are shown pre and post SNS-301 immunotherapy in these tables. A study is ongoing to compare efficacy and immunogenicity of 6 months off vs. 6 months on therapy.

TABLE 7
Changes in clinical activity parameters correlate
with antibody responses and ASPH-specific
B-cell and T-cell activation in individual patients

| | Pre- | Post- | Effect or |
|---|---|---|---|
| Patient Number: 003-002 | Vaccination | Vaccination | Fold-increase |
| PSADT/PSAV | 3.5/0.0065 | 16.1/0.0004 | 4.6 |
| ASPH-Specific CD4⁺ T-cells | 0% | 0.94% | Increased |
| (% of Total CD4⁺ T-cells, | | | |
| cycle 3) | | | |
| ASPH-Specific CD8⁺ | 0% | 0.89% | Increased |
| T-cells (% of Total CD8⁺ | | | |
| T-cells, cycle 3) | | | |
| ASPH-Specific B-Cells | 4.70% | 32.0% | 6.8 |
| (% Total B-Cells, cycle 3) | | | |
| Anti-ASPH Antibody | 0 | 76 | Increased |
| Titer (units/ml) | | | |

TABLE 8
Changes in clinical activity parameters correlate with antibody responses
and ASPH-specific B-cell and T-cell activation in individual patients

| | Pre- | Post- | Effect or |
|---|---|---|---|
| Patient Number: 004-004 | Vaccination | Vaccination | Fold-increase |
| PSADT/PSAV | 7.4/0.003 | Negative/ | Improved/ |
| | | −0.0017 | Decreased |
| ASPH-Specific CD4⁺ T-cells (% | 0.27% | 1.70% | 6.3 |
| of Total CD4⁺ T-cells, cycle 5) | | | |
| ASPH-Specific CD8+ T-cells (% | 0.25% | 2.10% | 8.4 |
| of Total CD8⁺ T-cells, cycle 5) | | | |
| ASPH-Specific B-Cells (% Total | 5.90% | 11.6% | 2.0 |
| B-Cells, cycle 5) | | | |
| Anti-ASPH Antibody Titer | 0 | 61 | Increased |
| (units/ml) | | | |

Longer-term immune responses were measured within the first 20 cycles of SNS-301 immunotherapy treatment. Longer-term immune responses measured included anti-ASPH antibody titers, percentages of ASPH-specific B-cells and production of anti-phage antibodies.

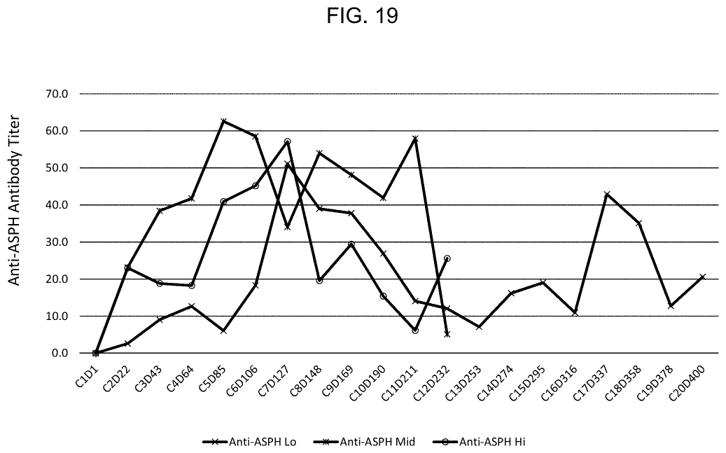

FIG. 19 shows anti-ASPH antibody levels measured in three cohorts of patients after treatment with SNS-301 immunotherapy. Anti-ASPH antibody levels were measured every 3 weeks at dosing using a tumor cell-based immunoassay. “Anti-ASPH Lo” refers to patients administered 2×10¹⁰ particles every 21 days for 3 doses (n=3). “Anti-ASPH Mid” refers to patients administered 1×10¹¹ particles every 21 days for 3 doses (n=3). “Anti-ASPH Hi” refers to patients administered 3×10¹¹ particles every 21 days for 3 doses (n=6). Arrows indicate peak antibody titers. The subsequent drop in antibody titers may be indicative of immune exhaustion. The mid-dose cohort showed the longest period of sustained anti-ASPH antibody in patient serum during treatment.

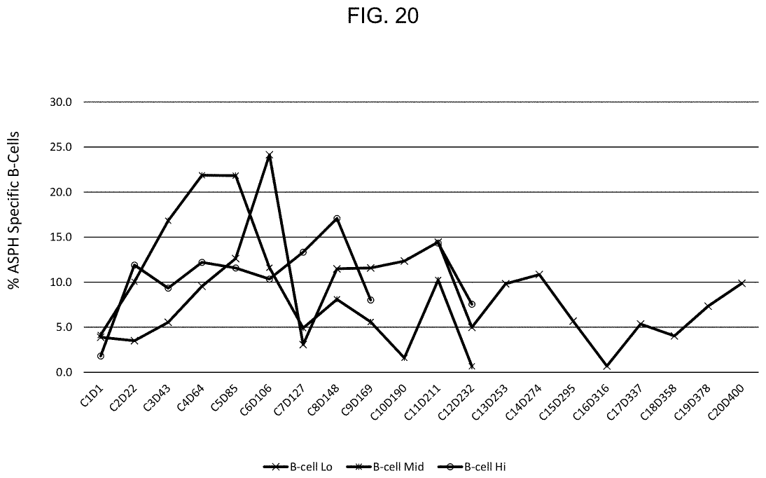

FIG. 20 shows ASPH-specific B-cell responses in three cohorts of patients after treatment with SNS-301 immunotherapy. ASPH-specific B-cells were assessed by flow cytometry using fluorescently labeled recombinant ASPH protein. Assessments were from PBMCs collected every 3 weeks at dosing. “B-cell Lo” refers to patients administered 2×10¹⁰ particles every 21 days for 3 doses (n=3). “B-cell Mid” refers to patients administered 1×10¹¹ particles every 21 days for 3 doses (n=3). “B-cell Hi” refers to patients administered 3×10¹¹ particles every 21 days for 3 doses (n=6). Arrows indicate peak B-cell responses. The subsequent drop in specific B-cells may be indicative of immune exhaustion. The mid-dose cohort had the earliest rise in ASPH specific B-cells.

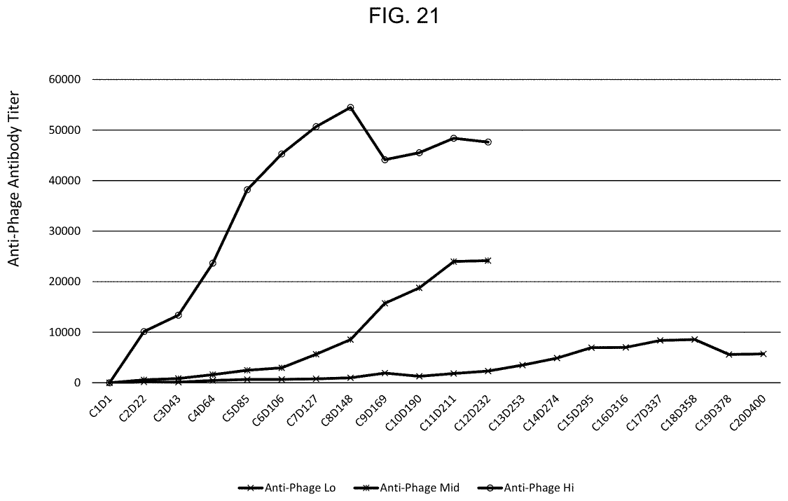

FIG. 21 shows anti-phage antibody titers in three cohorts of patients after treatment with SNS-301 immunotherapy. Anti-phage antibody levels were measured every 3 weeks at dosing using a tumor cell-based immunoassay. “Anti-Phage Lo” refers to patients administered 2×10¹⁰ particles every 21 days for 3 doses (n=3). “Anti-Phage Mid” refers to patients administered 1×10¹¹ particles every 21 days for 3 doses (n=3). “Anti-Phage Hi” refers to patients administered 3×10¹¹ particles every 21 days for 3 doses (n=6). The low-dose and mid-dose cohorts had significantly lower levels of anti-phage antibodies. The later rise in anti-phage antibody titers in these cohorts were correlated to the time at which these patients were switched to the high dose.

All patients experienced dose-dependent ASPH-specific immune responses including B-cell, T-cell and antibody responses, demonstrating that SNS-301 is capable of breaking immune self-tolerance to ASPH. Anti-ASPH antibody titers showed an initial increase in a dose-dependent fashion over the first 4-6 cycles (80-120 days) of SNS-301 administration. After an initial peak, titers dropped and then fluctuated up and down. At the low dose and the mid dose of SNS-301, ASPH-specific B-cell levels peaked at close to 20% of total B-cells at cycle 6 and 4, respectively, and subsequently dropped and then fluctuated up and down. At the high dose of SNS-301, ASPH-specific B-cell levels increased rapidly, but only to a maximum level of 10-15% of total B-cells. B-cell levels peaked prior to anti-ASPH antibody titers, which makes sense given that the B-cells are antibody-producing cells. The drop in ASPH-specific immune responses and subsequent fluctuation is suggestive of immune fatigue likely resulting from too frequent dosing of patients. Anti-phage antibody responses generally increased over the entire treatment period, however, were much lower at the low dose and mid dose than at the high dose. Furthermore, there was a significant lag period in this rise (past cycle 6 for mid dose and past cycle 11 for the low dose). This later rise correlates to the time at which low and mid dose patients were converted to the high dose.

In this phase 1 setting, the SNS-301 vaccine induced antigen-specific immune responses, which generally correlated with biochemical responses. The mid dose gave the earliest peak response to ASPH with relatively low anti-phage antibody titers and is thus recommended as phase 2 dose. Further, the data demonstrates a certain amount of immune fatigue suggesting increased spacing in time of boosting doses after the first 6 cycles.

TABLE 9
HAAH Construct sequences

MAP HAAH-Construct Ia
DRAMAQRKNAKSSGNSSSSGSGSGSTSAGSSSPGARRETKHGGHKNGR
KGGLSGTSFFTWFMVIALLGVWTSVAVVWFDLVDYEEVLGKLGIYDAD
GDGDFDVDDAKVLLGLKERSTSEPAVPPEEAEPHTEPEEQVPVEAEPQ
NIEDEAKEQIQSLLHEMVHAEHVEGEDLQQEDGPTGEPQQEDDEFLMA
TDVDDRFETLEPEVSHEETEHSYHVEETVSQDCNQDMEEMMSEQENPD
SSEPVVEDERLHHDTDDVTYQVYEEQAVYEPLENEGIEITEVTAPPED
NPVEDSQVIVEEVSIFPVEEQQEVPP
(SEQ ID NO: 1)
MAP HAAH-Construct II
LDAAEKLRKRGKIEEAVNAFKELVRKYPQSPRARYGKAQCEDDLAEKR
RSNEVLRGAIETYQEVASLPDVPADLLKLSLKRRSDRQQFLGHMRGSL
LTLQRLVQLFPNDTSLKNDLGVGYLLIGDNDNAKKVYEEVLSVTPNDG
FAKVHYGFILKAQNKIAESIPYLKEGIESGDP
(SEQ ID NO: 2)
MAP HAAH-Construct III
GTDDGRFYFHLGDAMQRVGNKEAYKWYELGHKRGHFASVWQRSLYNVN
GLKAQPWWTPKETGYTELVKSLERNWKLIRDEGLAVMDKAKGLFLPED
ENLREKGDWSQFTLWQQGRRNENACKGAPKTCTLLEKFPETTGCRRGQ
IKYSIMHPGTHVWPHTGPTNCRLRMHLGLVIPKEGCKIRCANETRTWE
EGKVLIFDDSFEHEVWQDASSFRLIFIVDVWHPELTPQQRRSLPAI
(SEQ ID NO: 3)
MAPHAAH-Construct I
STSEPAVPPEEAEPHTEPEEQVPVEAEPQNIEDEAKEQIQSLLHEMVH
AEHVEGEDLQQEDGPTGEPQQEDDEFLMATDVDDRFETLEPEVSHEET
EHSYHVEETVSQDCNQDMEEMMSEQENPDSSEPVVEDERLHHDTDDVT
YQVYEEQAVYEPLENEGIEITEVTAPPEDNPVEDSQVIVEEVSIFPVE
EQQEVPP
(SEQ ID NO: 5)

### Example 4: Phase 2 Clinical Trial of Cancer Vaccine Targeting Human Aspartyl (Asparaginyl) β-Hydroxylase in Patients with High Risk Myelodysplastic Syndrome and Chronic Myelomonocytic Leukemia

### Summary of Phase 2 Clinical Trial

An open-label, multi-center phase 2 clinical trial evaluating the cancer vaccine (SNS-301), which targets human aspartyl (asparaginyl) β-hydroxylase (alternatively abbreviated as HAAH or ASPH), in patients with high risk myelodysplastic syndrome (MDS) and chronic myelomonocytic leukemia (CMML) will be performed. The formulation of SNS-301 will be 1×10¹¹ particles in 1 mL intradermal (ID) injection.

### Study Design

Trial Population

Approximately 20 patients with ASPH+ high risk MDS and CMML (≤5/20 patients) will be enrolled in up to 15 institutions within the United States. The trial population will be divided into two groups: 1) MDS: satisfaction of Revised International Prognostic Scoring System (IPSS-R) criteria for categorization≥Intermediate Risk-3 (IR-3), and 2) CMML: satisfaction of World Health Organization (WHO) criteria for CMML-2, characterized by peripheral blasts of 5% to 19%, and 10% to 19% bone marrow blasts and/or presence of Auer rods.

Patient Enrollment

After consenting to participate in this clinical trial, participants will be screened for enrollment. The patient must have measurable ASPH expression in fresh bone marrow aspirate by flow cytometry to be eligible for the trial. An archival sample (aspirate or biopsy) will be requested, if available, for research purposes. On-treatment bone marrow aspirations/biopsies will be collected on day 42 (+/−3 days), at Week 12 and then every 12 weeks and as indicated at the discretion of the investigator until documentation of response. For patients who respond and subsequently progress as determined per International Working Group (IWG) criteria, a bone marrow aspirate/biopsy will be obtained at the time of disease progression at the discretion of the investigator.

The following procedures will be performed for all patients: (1) For eligible patients, the study treatment of SNS-301 will commence on Day 0 (first dose). (2) A fresh bone marrow aspirate will be collected at the time of screening to determine ASPH expression eligibility. A bone marrow aspirate/biopsy will be collected at 42 days (±3 days), Week 12 then every 12 weeks until documentation of disease response or first evidence of disease progression if clinically feasible. Patients who are unable to undergo bone marrow aspirate/biopsy sample collection but otherwise meet criteria listed in the protocol may continue to receive study treatment. A bone marrow biopsy is acceptable with the exception of screening where a bone marrow aspirate is required for flow cytometry. (3) SNS-301 will be administered ID using the 3M® hollow microstructured transdermal system (hMTS) every 3 weeks (±3 days) for 4 doses then every 6 weeks (+3 days) for 6 additional doses, thereafter every 12 weeks (+3 days) until confirmed disease progression, unacceptable toxicity, deemed intolerable by the investigator or up to 24 months in patients without disease progression. Survival follow up will be for three years after the patient discontinues treatment. The maximum amount of time that the patient will be on study is five years. (4) In patients who discontinue trial therapy for any reason other than confirmed disease progression, a bone marrow assessment should be performed at the time of treatment discontinuation (+4 weeks). If previous assessment was obtained within 4 weeks prior to the date of discontinuation, then additional assessment at treatment discontinuation is not required. (5) Patients will be followed for all adverse events (AEs) for 30 days and for adverse events of special interest (AESI) and serious adverse events (SAEs) occurring up until 90 days after the last dose of study treatment or until the start of a new anti-cancer treatment, whichever comes first. If the Investigator becomes aware of an AESI or SAE that is considered related to study treatment after discontinuation from the trial, those events should be reported to the Sponsor within 24 hours. (6) All patients who experience disease progression, have unacceptable toxicity or start a new anti-cancer therapy and are discontinued from the trial will be followed for survival and subsequent anti-cancer therapy. Patients should be contacted (i.e. by telephone) every 3 months to assess for survival status for up to 3 years until death or patient withdraws consent. A pregnancy test is also required for patients of WOCBP every 3 months. (7) Patients who discontinue from study treatment for reasons other than disease progression (e.g., toxicity) will continue scheduled tumor assessments until disease progression, withdrawal of consent, or start of new anti-cancer therapy, death, or trial termination by Sponsor, whichever occurs first.

Study Treatment

### Statistical Methods

Patients with MDS and CMML will be reviewed together as the ITT population as well as analyzed separately. AEs, based on CTCAE v5.0, will be recorded by AE name, grade, and attribution to treatment, with start and resolution dates. AEs will be summarized using frequencies, percentages and confidence intervals.

Sample Size

Approximately 20 patients with ASPH+ high risk MDS and CMML (≤5/20 patients) will be enrolled in up to 15 institutions within the United States.

Safety

The safety analysis will be based on the Intent-to-Treat (ITT)/Safety Population, which comprises all participants who receive at least 1 dose of the study treatment. Safety and tolerability will be assessed through AEs, clinical laboratory parameters, vital signs, and physical examination finding.

Efficacy

The primary efficacy analysis will be performed on the ITT/Safety population. The PP population will be the subset of the Safety Population that is compliant with the protocol and excludes subjects with major protocol violations and have at least 1 post baseline efficacy response assessment per IWG 2006 criteria. The protocol violation criteria will be defined in the SAP. Analyses of efficacy variables will be performed on subgroups of interest (MDS & CMML) and will be outlined in the SAP.

Immunology Population

Immunology Population: All patients who receive at least 1 dose of the study treatment and have at least one valid post baseline immunologic assessment available will be analyzed in the Immunology Population.

### Trial Schedule

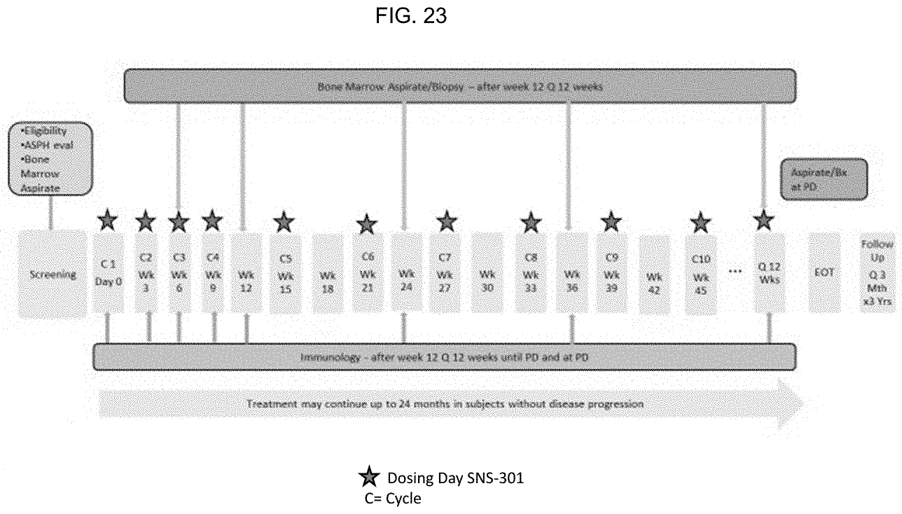

Table 10 illustrates the schedule of events for the Phase 2 clinical trial. FIG. 23 shows a timeline of the dosing and administration of SNS-301.

TABLE 10
Trial Schedule of Events

| | | Cycle | Cycle | Cycle | Cycle | | | | Dis- | |
|---|---|---|---|---|---|---|---|---|---|---|
| | Screening^{a} | 1 | 2 | 3 | 4 | Every | Every | Every | continu- | |
| | Day −28 to | Day | Week | Week | Week | 3 | 6 | 12 | ation | Follow- |
| | Day −1 | 0 | 3 | 6 | 9 | weeks | weeks | weeks | Visit^{b} | up |
| Signed | X | | | | | | | | | |
| Informed | | | | | | | | | | |
| Consent | | | | | | | | | | |
| Form(s) | | | | | | | | | | |
| Medical, | X | | | | | | | | | |
| surgical, | | | | | | | | | | |
| and | | | | | | | | | | |
| cancer | | | | | | | | | | |
| histories, | | | | | | | | | | |
| including | | | | | | | | | | |
| demographic | | | | | | | | | | |
| Inclusion/ | X | X | | | | | | | | |
| Exclusion | | | | | | | | | | |
| criteria | | | | | | | | | | |
| Complete | X | | | | | | | | | |
| Physical | | | | | | | | | | |
| exam^{d} | | | | | | | | | | |
| Targeted | | X^{e} | X^{e} | X^{e} | X^{e} | X^{e} | | | X | |
| Physical | | | | | | | | | | |
| exam^{e} | | | | | | | | | | |
| ECOG | X | X | X | X | X | X | | | X | |
| performance | | | | | | | | | | |
| HIV, Hep B | X | | | | | | | | | |
| and Hep C | | | | | | | | | | |
| serology^{f} | | | | | | | | | | |
| Concomitant | X | X | X | X | X | X | | | X | |
| Medications^{g} | | | | | | | | | | |
| Anticoagulant | X | X | X | X | X | After week 9, | | | X | |
| specific drug | | | | | | Q6w until | | | | |
| and/or | | | | | | week 45, | | | | |
| anticoagulant | | | | | | thereafter | | | | |
| factor Xa | | | | | | Q12w | | | | |
| levels^{h} | For patients on anticoagulants only | | | | | | | | | |
| Vital Signs | X | X | X | X | X | X | | | X | |
| and Weightⁱ | | | | | | | | | | |
| Height | X | | | | | | | | | |
| 12-lead | X | | | | | | | | | |
| ECGP^{j} | | | | | | | | | | |
| IWG | X | | | X | At week 12 and Q12w thereafter | | | | | |
| Assessment | | | | | until disease progression as well | | | | | |
| | | | | | as at disease progression | | | | | |
| Hematology^{k} | X | X^{e} | X^{e} | X^{e} | X^{e} | X^{e} | | | X | |
| Serum | X | X^{e} | X^{e} | X^{e} | X^{e} | X^{e} | | | X | |
| Chemistry^{l} | | | | | | | | | | |
| Coagulation | X | | | | | | | | | |
| Panel | | | | | | | | | | |
| (aPTT, INR) | | | | | | | | | | |
| Urinalysis^{m} | X | | | X | | | X | | X | |
| Urineⁿ | | X | X | | X | Week 18, week 27, | | | | |
| | | | | | | week 36, week 45 and | | | | |
| | | | | | | disease progression | | | | |
| Pregnancy^{o} | X | X | X | X | X | X | | | X | X |
| CPK | X | | | | | | | | X | |
| Plasma, Serum | | X | X | X | X | At week 12 and | | | | |
| and Whole | | | | | | Q12w thereafter until | | | | |
| blood ample | | | | | | disease progression | | | | |
| for | | | | | | as well as at | | | | |
| immunology^{p} | | | | | | disease progression | | | | |
| Bone | X | | | X | At week 12 and Ql2w thereafter | | | | | |
| marrow | | | | | until disease progression | | | | | |
| aspirate/ | | | | | as well as at | | | | | |
| biopsy^{q} | | | | | disease progression | | | | | |
| Adverse Events^{r} | X | X | X | X | X | X | X | X | X | X |
| SNS-301^{s} | | X | X | X | X | After week 9, Q6w | | | | |
| | | | | | | for 6 more doses | | | | |
| | | | | | | (week 45), | | | | |
| | | | | | | thereafter Q12w | | | | |
| | | | | | | until PD or 24 | | | | |
| | | | | | | months if no PD | | | | |
| Survival | | | | | | | | | | X |
| and new | | | | | | | | | | |
| anti-cancer | | | | | | | | | | |
| therapy | | | | | | | | | | |
| follow-up^{t} | | | | | | | | | | |

Note:
Assessments scheduled on the days of study treatmentshould be performed before the study treatment unless otherwise noted. “Q” refers to “every”. “w” refers to “weeks”.
^{a}Written informed consent can be obtained up to 28 days prior to Day 0 and is required for performing any trial-specific tests or procedures. Biopsy sample maybe submitted up to 28 days prior to Day 0. Results of standard-of-care tests or examinations performed prior to obtaining informed consent and within 28 days prior to Day 0 may be used for screening assessments rather than repeating such tests. Screening labs (CBC and chemistry) may be used for Day 0 if they are within 10 days of Day 0.
^{b}Patients who discontinue early from study treatment for progression (i.e., progression, adverse event, etc.) will be asked to return to the clinic within 30 days after the last dose for a treatment discontinuation visit.
^{c}Cancer history includes stage, date of diagnosis, and prior anti-tumor treatment. Previous progression data will be collected as well. Demographic information includes sex, age, and self-reported race/ethnicity. Reproductive status and smoking/alcohol history should also be captured.
^{d}A complete physical exam will include head, eyes, ears, nose, throat and cardiovascular, dermatological, musculoskeletal, respiratory, gastrointestinal and neurological systems. Height and weight will also be collected. Any signs and symptoms, other than those associated with a definitive diagnosis, should be collected at baseline during the study. A targeted, symptom-directed exam will be performed as clinically indicated.
^{e}PECOG performance status, targeted physical exam, and local laboratory assessments may be obtained <72 hours before each dosing visit.
^{f}Patients should be tested for HIV locally prior to the inclusion into the trial if the investigator suspects HIV infection and HIV-positive patients will be excluded from the clinical trial. Hepatitis B surface antigen, anti-HBc antibody, anti-HBs antibody, and Hepatitis C antibody immunoassays should be tested only per investigator's clinical suspicion during screening and tested locally. In patients who have positive serology for the anti-HBc antibody, HBV DNA should be tested prior to Day 0.
^{g}Concomitant medications include any prescription medications or over-the-counter medications. At screening, any medications the patient has used within the 7 days prior to the screening visit should be documented. At subsequent visits, changes to current medications or medications used since the last documentation of medications will be recorded.
^{h}Specific anticoagulant drug and/or anticoagulant factor Xa levels will be obtained only on patients receiving anticoagulant therapy. Drug levels will also be obtained at any time of clinical bleeding. Traditional testing methods can be used for warfarin, heparin (e.g., PT/INR, aPTT, TT). Novel oral anticoagulants may require anticoagulant factor Xa levels or anticoagulant drug specific level testing.
ⁱVital signs include heart rate, respiratory rate, blood pressure and temperature. For the first injection, the subject's vital signs should be determined within 60 minutes before the injection. Vital signs should be recorded at 30 (±5) minutes after the injection.
^{j}ECG recordings will be obtained during screening and as clinically indicated at other time points. Patients should be resting and in a supine position for at least 10 minutes prior to ECG collection.
^{k}Hematology consists of CBC, including RBC count, hemoglobin, hematocrit, WBC count with automated differential (absolute counts of neutrophils, lymphocytes, eosinophils, monocytes, basophils, and other cells (if any)), and platelet count. A manual differential should be done. Peripheral blast counts will also be collected.
^{l}Serum chemistry includes BUN or urea, creatinine, sodium, potassium, magnesium, chloride, bicarbonate or CO2, calcium, phosphorus, glucose, total bilirubin (direct bilimbin only if total bilirubin is elevated), ALT, AST, alkaline phosphatase, lactate dehydrogenase, total protein, and albumin.
^{m}Urinalysis includes specific gravity, pH, glucose, protein, ketones, blood, and a microscopic exam if abnormal results are noted. Urinalysis to be performed every 6 weeks.
ⁿA urine sample will be collected at Day 0, week 3, week 9, week 18, week 27, week 36, week 45 and disease progression.
^{o}Serum pregnancy test (for women of childbearing potential, including women who have had a tubal ligation) must be performed and documented as negative within 72 hours prior to each dose.
^{p}Immunology samples and bone marrow assessments are to be drawn at screening (after the patient has been deemed ASPH+), Week 3, Week 6, Week 9, Week 12 and thereafter every 12 weeks until disease progression, as well as at disease progression and discontinuation visit.
qA pre-treatment fresh bone marrow aspirate sample will be analyzed for ASPH expression as part of the screening process. After signing of the Informed Consent Form, bone marrow aspirate and archival sample(s) should be submitted in a timely manner. The bone marrow aspirate sample will be collected and analyzed preferably before other non-SOC procedures. Eligibility based on ASPH expression will be provided back to the sites within 5-8 business days. An archival sample (aspirate or biopsy), ideally treatment naive will be requested, if available. Bone marrow aspirate/biopsy assessments will be collected at 42 days (3 days), Week 12 then after approximately 12 weeks until documentation of disease response or first evidence of disease progression if clinically feasible. Patients who are unable to undergo bone marrow aspirate/biopsy sample collection but otherwise meet criteria listed in the protocol may continue to receive study treatment.. A bone marrow biopsy is acceptable with the exception of screening where a bone marrow aspirate is required for flow cytometry.
^{r}PAEs will be collected from the time of informed consent until 30 days after the last dose of study treatment or until initiation of another anti-cancer therapy, whichever occurs first. SAEs and AESIs will be collected from the time of informed consent until 90 days after the last dose of study treatment of until initiation of anti-cancer therapy, whichever occurs first.
^{s}SNS-301 is administered every 3 weeks until week 12 (ie.,4 doses). Then every 6 weeks for 6 more doses (until week 45). Thereafter it will be administered every 12 weeks until confirmed disease progression, unacceptable toxicity, deemed intolerable by investigator or up to 24 months in patients without disease progression. The window for each visit is 3 days unless otherwise noted. For the first injection, the patient should be observed for 60 minutes. For subsequent injections, a 30-minute observation period is recommended after each study treatment.
^{t}Survival follow-up information will be collected via telephone calls, patient medical records, and/or clinical visits approximately every 3 months for up to 3 years until death, lost to follow-up, withdrawal of consent, trial termination by Sponsor. All patients will be followed for survival and new anticancer therapy information unless the patient requests to be withdrawn from follow-up; this request must be documented in the source documents and signed by the investigator. If the patient discontinues study treatment without documented clinical disease progression, every effort should be made to follow up regarding survival, progression (if not already progressed), and new anti-cancer therapy.

### Detailed Summary of Phase 2 Clinical Trial

### Pharmaceutical and Therapeutic Background

Human aspartyl-asparaginyl-β-hydroxylase (HAAH), also known as aspartate-β-hydroxylase (ASPH), is an ˜86 kDa type 2 transmembrane protein that belongs to the α-ketoglutarate-dependent dioxygenase family (Jia, S. et al. 1992. The Journal of Biological Chemistry 267:14322-14327). It is a highly conserved enzyme, which catalyzes the hydroxylation of aspartyl and asparaginyl residues in epidermal growth factor-like domains of proteins including Notch and homologs (Lavaissiere, L. et al. 1996. The Journal of Clinical Investigation 98:1313-1323). ASPH was initially identified in a novel screen to identify cell surface proteins up-regulated in hepatocellular carcinoma. It has subsequently been detected in a diverse array of solid and blood cancers, including: liver, bile duct, brain, breast, colon, prostate, ovary, pancreas, and lung cancers as well as various leukemias (Table 11). ASPH is not found in significant quantities in normal tissue or in proliferative disorders.

TABLE 11
ASPH Expression

| | ASPH % Positive Expression (Number Tested) | | | | |
|---|---|---|---|---|---|
| | IHC of Tissue | | Serum | | Flow |
| Tumor Type | Samples | | ELISA | | Cytometry |
| Normal Bone Marrow | 0% | (130) | NT | | NT |
| Breast | 85% | (47) | 94% | (181) | NT |
| Cholangiocarcinoma | 100% | (27) | NT | | NT |
| Colon Cancer | 75% | (41) | 99% | (145) | NT |
| Gastric | 80% | (51) | NT | | NT |
| Glioblastoma | 98% | (15) | NT | | NT |
| Head and Neck | 91% | (22) | 75% | (12) | NT |

| | | | | |
|---|---|---|---|---|
| Hepatocellular | 92% | (87) | NT | NT |
| Carcinoma | | | | |
| Lymphoid Leukemia | 49% | (80) | NT | NT |

| | | | | | |
|---|---|---|---|---|---|
| MDS | NT | 50% | (10) | 91% | (11) |

| | | | | | |
|---|---|---|---|---|---|
| Mesothelioma | 100% | (3) | 100% | (12) | NT |

| | | | | | |
|---|---|---|---|---|---|
| Myeloid Leukemia | 88% | (79) | NT | 33% | (42) |

| Lung | 82% | (304) | 99% | (160) | NT |
|---|---|---|---|---|---|
| Osteosarcoma | 80% | (18) | NT | | NT |
| Pancreatic | 97% | (109) | NT | | NT |
| Prostate Cancer | 96% | (46) | 95% | (233) | NT |

| | | | | |
|---|---|---|---|---|
| Renal cancer | 83% | (49) | NT | NT |
| Soft Tissue Sarcoma | 84% | (30) | NT | NT |

NT = not tested

Over-expression of ASPH has been demonstrated to be sufficient to induce cellular transformation, increase cellular proliferation and cellular motility while suppression of ASPH expression (small interfering ribonucleic acid) or neutralized activity (monoclonal antibodies) returns cancer cells to a normal phenotype. In cancer cells, ASPH has been shown to be translocated to the cellular surface where it is not normally located. Because ASPH is an embryonic antigen, and as such presents self-antigen tolerance, it is difficult to elicit a robust immune response against it and break immune tolerance. Thus, we hypothesized that effective priming of antigen-presenting cells (APC) by ASPH antigen is an essential step to overcome immune tolerance. Indeed, in vitro activation of dendritic cells with ASPH, prior to re-administration to patients with hepatocellular carcinoma has been performed (Shimoda, M. et al. 2012. Journal of Hepatology 56:1129-1135).

Bacteriophage offers a simple, inexpensive and practical way of achieving favorable presentation of peptides to the immune system. The phage contains deoxyribonucleic acid (DNA) fragments that present the phage CpG motifs, which are known to stimulate the innate immune response and activate the major histocompatibility class II (MHC-II) pathway in APC. Previous findings have revealed that recombinant bacteriophage can prime strong CD8+ T-lymphocyte (CTL) responses both in vitro and in vivo against epitopes displayed in multiple copies on their surface, activate helper T cells and elicit the production of specific antibodies without requiring any exogenous adjuvants. (De Berardinis, P. et al. 2000. Nature Biotechnology 18(8):873-876.; De Berardinis, P. et al. 1999. Vaccine 17 (11-12):1434-1441.; Di Marzo et al. 1994. Journal of Molecular Biology 243(2):167-172.; Perham, R. 1995. FEMS Microbiology Reviews 17(1-2):25-31.)

Thus, we have selected bacteriophage as a platform for eliciting anti-ASPH immune responses. Bacteriophages are ubiquitous and essentially innocuous to humans, however, as an added safety mechanism, they may be neutralized rendering them non-infective to host bacteria while retaining their immunostimulant properties. Once neutralized, the bacteriophage effectively becomes a nanoparticle, for enhanced delivery of protein fragments to APC.

We have designed a bacteriophage lambda system to display ASPH peptides fused at the C terminus of the head protein gpD of phage lambda. The phages carry 200-300 copies of the gpD protein on their head and thus display many copies of an approximately 25 kDa molecular weight fragment of ASPH on their surface. The drug substance is one of these ASPH bacteriophage lambda constructs: HAAH-1λ (SNS-301).

### Pre-Clinical, Clinical Trials, and Other Ongoing Trials

The following paragraphs describe pre-clinical experiments, previous clinical trials, and other ongoing trials.

Pre-Clinical

Nonclinical studies have focused on the immunogenicity and efficacy of the ASPH Nanoparticle Vaccine in rodent models. Nonclinical toxicology studies have been completed as well.

The ASPH Nanoparticle Vaccine was administered 3 times in rodent models, at a dosing frequency of weekly in mice and every 3 weeks in rats. Demonstration of immunogenicity in mice and rats was accomplished by showing ASPH-specific activation of both humoral and cellular immunity. A dose response to the amount of vaccine delivered as well as to the number of doses given was observed. In rats, the intradermal vs intramuscular routes of administration were evaluated. ASPH-specific immunogenicity was clearly superior with intradermal delivery. Antibodies to the lambda bacteriophage portion of the vaccine are generated in a dose level- and dose number-dependent manner but appear to have no negative (neutralizing) effect on the immunogenicity of subsequent doses of the vaccine.

Efficacy was evaluated in multiple studies in immune-competent rodent tumor models by examining tumor growth and metastatic potential. Two mouse models were evaluated, one using the BNLT3 cell line, a BALB/c-derived hepatocellular carcinoma cell line that produces solid tumors when administered subcutaneously and metastatic tumors when injected into the spleen or peritoneum, and the BALB/c-derived breast cancer cell line, 4T1, that is injected into the mammary gland and typically forms both a solid tumor and metastases in other organs, such as the lung. In each model, animals were injected with tumor cells prior to, or simultaneous with, the first injection of ASPH Nanoparticle Vaccine. Solid tumor growth was significantly reduced in vaccinated compared to control animals in all studies. Likewise, BNLT3 peritoneal metastases and 4T1 lung metastases were significantly reduced in vaccinated animals. A rat model of prostate cancer was also evaluated using the MLLB-2 cell line that is derived from Copenhagen rats and can cause hind limb paralysis due to metastasis. Hind limb paralysis was reduced by ⅔ in vaccinated compared to control animals.

In the experiments described above, there were no local reactogenicity or adverse events (AEs) associated with the administration of multiple doses of the ASPH Nanoparticle Vaccine in both mice and rats. While these vaccines were immunogenic and showed efficacy in three tumor model systems, they were safe in the doses given to rodents. These data strongly support the potential utility and expected safety of using the SNS-301 vaccine in patients for cancer immunotherapy.

A repeated dose study in rats has been conducted that assessed the toxicity of the SNS-301 (previously named PAN-301-1) ASPH nanoparticle vaccine when administered intradermally at the same three dose levels (2×10¹⁰, 1×10¹¹ and 3×10¹¹ particles) and same dose schedule (3 doses given at 21 day intervals) as was ultimately undertaken in the Phase 1 human study (SNS0216), followed by 2 week and 4 week recovery periods. Treatment with SNS-301 at doses up to 3×10¹¹ particles had no effect on mortality, physical examinations, cage-side observations, dermal Draize observations, body weights or body weight changes, food consumption, body temperature, ophthalmologic observations, gross pathology, absolute and relative organ weights, hematology or clinical chemistry. A slightly prolonged but non-adverse prothrombin time (PT) in males given >1×10¹¹ particles and females given 3×10¹¹ particles persisted through the first recovery period (2 weeks post-3rd injection). The prolonged PT resolved in males by the second recovery period (4 weeks post-3rd injection) but remained minimally prolonged in females given 3×10¹¹ particles. Test article-related microscopic findings were present in the injection site in animals given >1×10¹¹ particles and consisted of mild or moderate mononuclear or mixed inflammatory cell infiltrates in the dermis and/or subcutis. These findings were considered non-adverse and resolved during recovery. Additionally, the SNS-301 vaccine demonstrated a significant antibody response that was dependent on both the dose level and number of doses administered.

The toxicology results demonstrate safety of the SNS-301 ASPH nanoparticle vaccine and supported the multiple dose Phase 1 study (SNS0216) of the ASPH Nanoparticle Vaccine in humans that was subsequently completed.

The Sponsor completed a Phase 1 clinical study (SNS0216, previously PAN0216), in Biochemically Recurrent Prostate Cancer (BRPC) patients which was a 3+3 dose-escalation study (also described in Example 3), where the starting dose and the subsequent dose escalations were determined from the results of a repeated dose toxicology study conducted previously in rats and was described above.

The selection rationale for the specific dose levels evaluated in the Phase 1 study was based on the in vivo results of this range of doses in mice and rats that have shown both immunogenicity and efficacy of the SNS-301 vaccine. The vaccine has demonstrated immunogenicity in rats by IgG antibody response to recombinant ASPH and to the recombinant bacteriophage ASPH-1λ drug substance at each dose level, 2×10¹⁰, 1×10¹¹ and 3×10¹¹ particles. This antibody response was both dose level-dependent and dose number-dependent. In the SNS0216 patients, a significant percentage of ASPH-specific B-cells and high levels of anti-ASPH specific antibodies were detected in patient peripheral blood; these levels seemed to plateau at the 1×10¹¹ dose. Anti-phage antibodies could also be detected at all dose levels but were significantly higher at the 3×10¹¹ particle dose than at the 1×10¹¹ particle dose. Despite the high levels of anti-phage antibodies, these antibodies did not neutralize further doses of vaccine. As previously described, the SNS-301 vaccine has demonstrated inhibition of solid tumor growth and metastases in in vivo mouse tumor models with the mouse hepatocellular carcinoma cell line, BNLT3, and with the mouse breast cancer cell line, 4T1 and have demonstrated inhibition of metastases in a rat tumor model with the rat prostate cancer cell line, MLLB-2.

The SNS-301 dose and schedule selected for this study (1×10¹¹ dose/1 mL) ID injection using the 3M® hMTS device to be administered every 3 weeks (+3 days) until week 12 (i.e., 4 doses) then every 6 weeks for 6 more doses (until week 45). Thereafter, it will be administered every 12 weeks until confirmed disease progression, unacceptable toxicity, deemed intolerable by investigator or up to 24 months in patients without disease progression, as based on the Phase 1 Study SNS0216 safety, immunogenicity and efficacy study.

Review of Current Clinical Data with SNS-301

### Rationale

Immunotherapy has become a pillar of cancer therapy along with surgery, radiation, chemotherapy and biologic targeted therapy. In this space, cancer vaccines are well suited to elicit a potent and focused immune response to lead to a clinically meaningful anti-tumor response.

### Rationale for the Trial and Selected Patient Population

SNS-301 is a cancer vaccine designed to generate functional cytotoxic T cells that traffic to the tumor and elicit a potent anti-tumor response leading to clinically meaningful improvements in patients with MDS/CMML who have failed standard of care (SoC) therapy with hypomethylating agents (HMA). In the setting of high-risk MDS/CMML, the overall survival (OS) remains low and there is a need to improve clinical outcomes for these patients. Sensei Bio plans to develop SNS-301 with the goal of improving OS for these patients.

### Unmet Medical Need

More than 15,000 new cases of MDS (pre-acute myeloid leukemia [AML]) are diagnosed each year in the United States and long-term survival is <5%. Siegel et al. C A Cancer J Clin. 2017 January; 67(1):7-30.; Cogle C. Curr Hematol Malig Rep (2015) 10: 272-281. Bejar R. Blood. 30 Oct. 2014. 124(18): 2794-2803. The only potentially curative approach is allogeneic stem cell transplantation.

However, due to advanced age and the presence of comorbid conditions, only ˜5% of diagnosed patients currently undergo this procedure. There are three drug therapies approved in the United States for MDS: the parenterally administered nucleoside analog DNA methyltransferase inhibitors (“hypomethylating agents”) Vidaza® (azacytidine) and Dacogen® (decitabine), and the orally administered “immunomodulatory” agent Revlimid® (lenalidomide). Azacitidine and decitabine are FDA-approved for all MDS risk groups, but are primarily used in higher-risk patients, and lenalidomide's approval is limited to transfusion-dependent lower-risk MDS with the 5q-chromosome abnormality. All 3 therapies are associated with treatment-emergent cytopenias and other adverse events, even in patients who achieve complete remission (CR) by conventional parameters, neoplastic stem cells persist in the marrow. Available drug therapies can induce hematologic improvement, but are not curative, and only azacitidine has been demonstrated to modestly improve survival in higher-risk patients (median 24 months with azacitidine versus 15 months for controls). Fenaux P F et al. 2009 (10): 223-232.; Steensma D. Mayo Clin Proc. 2015; 90(7): 969-983. Many MDS patients are also treated off-label with hematopoietic growth factors, which can provide some palliative benefit.

Patients with MDS for whom a hypomethylating agent has failed have a poor overall survival, with a median life expectancy of <6 months. Steensma D. Mayo Clin Proc. 2015; 90(7): 969-983. For these patients, options are limited and there are no agents known to increase survival in this setting, so most patients receive only supportive care. Furthermore, MDS progresses to AML in approximately 25% patients. These patients have an initial CR rate achieved after standard induction therapy between 45% and 60%. However, the probability of remaining in remission 3 years after diagnosis is below 10%, the median overall survival is 5-10 months, and the 5-year survival rate is 6-12%.

Under the French-American British (FAB) classification, Chronic Myelomonocytic Leukemia (CMML) has been interpreted as an MDS sub-category and, as such, constitutes approximately 10% of all MDS. Under the current World Health Organization (WHO) classification, however, it has been re-characterized as a new category of myelodysplastic myeloproliferative overlap syndromes. It is a clonal hematological malignancy characterized by increased peripheral and bone-marrow monocytes and blasts, ineffective hematopoiesis, and an increased risk of transformation to AML Padron E. et al. Clin Advances in Hem & Onc. 2014; 12(3): 172-178. Onida F. et al. Haematologica. 2013; 98(9): 1344-1352.

The prognosis of patients with CMML is poor overall, with a median survival of only 20 to 30 months and leukemia transformation rates of 15% to 20%. These survival rates compare unfavorably to MDS survival rates, suggesting that CMML is an even more aggressive disease.

Therapeutic options for CMML patients continue to be limited by the lack of CMML-specific trials. The treatment of CMML has progressed from cytotoxic chemotherapy with high toxicity and low response rates, with agents such as etoposide and hydroxyurea, to hypomethylating agents with higher response rates and lower toxicity. Allogeneic stem cell transplantation is the only strategy that may lead to cure in patients with CMML. However, due to the advanced age of the vast majority of patients (median age of 65-75 years), this treatment option is rarely feasible Padron E. et al. Clin Advances in Hem & Onc. 2014; 12(3): 172-178. Onida F. et al. Haematologica. 2013; 98(9): 1344-1352.

Like MDS, CMML represents an unmet medical need with significant need for clinically meaningful novel therapeutic approaches for these patients.

### ASPH Expression Testing in MDS/CMML

The sponsor has tested 42 acute myeloid leukemia bone marrow aspirates for cell surface expression of ASPH on blasts using flow cytometry. In that study, 14 (38%) samples were positive, including 4 of 4 (100%) of AML patients whose disease was preceded by MDS. In a separate study of 11 bone marrow aspirates obtained from individuals diagnosed with MDS, 10 out of 11 (91%) of samples were positive for ASPH expression. Of the 10 positive patients, six of the samples were taken at diagnosis and two were at relapse, one of which had been treated with 6 cycles of azacytidine, and one was a patient who was progressing after treatment with 5 cycles of azacytidine.

### Rationale for Translational Biomarkers

The sponsor has developed an analytical method to screen for ASPH expression in patients with MDS and AML. Flow cytometry for detection of ASPH on the surface of cancer cells in blood or bone marrow will be performed.

All ASPH (+) samples and a random sample of ASPH negative samples will be banked in the event that they may be necessary for future analysis or development of a companion diagnostic.

### Rationale for Dose Selection/Regimen

SNS-301 (1×10¹¹ particles in 1 ml ID injection) is planned to be administered every 3 weeks for 4 doses (12 weeks), and then every 6 weeks for 6 more doses (45 weeks). Thereafter, SNS-301 is to be administered every 12 weeks for up to 24 months or until confirmed disease progression, unacceptable toxicity, or deemed intolerable by the investigator.

The SNS-301 dose was chosen based on the safety, immunogenicity and preliminary efficacy data that were available from the Phase 1 dose escalation study (Study SNS0216) conducted in patients with biochemically relapsed prostate cancer.

As of the last cutoff date, regarding the overall safety profile, SNS-301 was considered to be tolerable with no dose limiting toxicities (DLTs) observed. There were no discernable safety differences noted across the 3 doses tested.

The immunogenicity of SNS-301 was evaluated for both antibody and cellular responses. At all dose levels tested, SNS-301 was able to generate specific anti-ASPH responses, however, the mid-dose level (and proposed Phase 2 clinical dose) demonstrated the best ASPH-specific antibody and cellular responses.

The clinical efficacy of SNS-301 was evaluated by examining PSA kinetics such as the effect of SNS-301 on PSA doubling time (PSADT), absolute PSA levels and PSA velocity (PSAV). A positive effect in lengthening the time in months to double the PSA value in the treatment phase compared to the pre-treatment phase was observed in 2 of 3 patients in both the low dose (2×10¹⁰ particles) and the mid dose (1×10¹¹ particles) treatment groups. In the high dose (3×10¹¹ particles) group, 3 of 6 patients showed a positive treatment effect.

In summary, SNS-301 was considered to be well tolerated at all dose levels evaluated with no DLTs or grade 4/5 AEs noted. Three patients experienced a total of five adverse events considered by investigators to be at least possibly related to study drug and all AEs were considered to ≤grade 3.

One patient experienced an AE in the form of migratory arthralgia that was attributed as possibly related to the study drug given that the patient was diagnosed with RF+ rheumatoid arthritis and the immunization contributed to the pain flare.

One patient 001-004 in the high dose cohort experienced mild erythema at the injection site which resolved within 3 days.

There was one serious treatment related TEAE reported. The patient (004-003), a 72-year-old, white male experienced positional vertigo that was deemed definitely not related by the reporting Investigator.

No other noteworthy AEs have been observed. The efficacy analysis showed a disease stabilizing effect of SNS-301 as evidenced by significant improvements in PSADT post-therapy for the patients. We believe that the excellent safety profile of SNS-301 coupled with the preliminary efficacy observed in patients with prostate cancer warrants further evaluation of SNS-301 at the mid-dose level of 1×10¹¹ in the unmet medical need patient population of patients with MDS/CMML.

### Benefit/Risk

The median OS of high risk MDS patients post-HMA failure is about 5.6 months with a 2-year OS of 15%. Therefore, this population represents a high unmet medical need and overall the clinical benefit potential outweighs the risks associated with SNS-301.

There have been no significant safety findings with no related Grade 3 or Grade 4 adverse events and no related serious adverse events with SNS-301. Of note, no studies assessing the reproductive and developmental toxicity of SNS-301 have been conducted to date. It is not known whether SNS-301 can cross the placenta or cause harm to the fetus when administered to pregnant women or whether it affects reproductive capacity. However, the antigen targeted by SNS-301 is involved in uterine implantation of the embryo. Therefore, SNS-301 should not be administered to pregnant women and pregnancy testing will be performed in women of childbearing potential (WOCBP) at screening and prior to each dose. WOCBP and male partners of such women should take necessary precautions to avoid pregnancy while receiving SNS-301, for the protocol defined period following the last dose of investigational product.

It is not known whether SNS-301 is excreted in human milk. Because of the unknown potential for serious adverse drug reactions in nursing infants, investigational product should not be administered to nursing mothers.

### Objectives and Endpoints

The primary objectives of the phase II trial will be to determine the safety and tolerability of SNS-301 delivered by intradermal injection (ID) using the 3M® hollow microstructured transdermal system (hMTS) device in patients with ASPH+ high risk MDS and CMML and to evaluate the anti-tumor activity of SNS-301 delivered by intradermal injection (ID) using the 3M® hollow microstructured transdermal system (hMTS) device in patients with ASPH+ high risk MDS and CMML. Associated endpoints to assess the primary objectives of the Phase 2 clinical trial will include evaluation of adverse events (AEs), as classified by the Common Terminology Criteria for Adverse Events (CTCAE) version 5.0. Adverse events will be evaluated using clinically significant changes in safety laboratory parameters from baseline. Safety laboratory parameters will include: complete blood count (CBC) with differential, chemistry panel, urinalysis, creatine phosphokinase (CPK), adverse events of special interest (AESI) classified by system organ class (SOC), preferred term (PT), and severity and relationship to drug. Criteria from the International Working Group in 2006 (IWG 2006), such as the objective response rate (ORR), minimal residual disease (MRD), duration of response (DoR), disease control rate (DCR), progression free survival (PFS), and overall survival (OS), will be measured.

A secondary objective of the phase II trial will be to evaluate the preliminary immune response to SNS-301 delivered by intradermal injection (ID) using the 3M® hollow microstructured transdermal system (hMTS) device in patients with ASPH+ high risk MDS and CMML. Associated secondary endpoints will include assessment of antigen-specific cellular immune responses. Non-limiting examples of antigen-specific cellular immune responses will include interferon-y secreting T lymphocytes in peripheral blood mononuclear cells (PBMCs) by ELISpot, assessment of T cell activation and cytolytic cell phenotype in PBMCS or secretion of immune molecules by flow cytometry or ELISpot, assessment of B cell activation and antibody secretion, assessment of myeloid derived suppressor cells (MDSCs), T cell receptor (TCR) sequencing of PBMCs for diversity and putative antigen specificity, immune gene transcript profiling of PBMCs, and assessment of proinflammatory and immunosuppressive elements in neoplastic and adjacent normal tissue, where feasible.

An exploratory objective of the phase II trial will be to evaluate tumor and immune biomarkers and their association with treatment outcome (antitumor activity and/or safety) in ASPH+ patients with high risk MDS and CMML. Associated exploratory endpoints will include immune related gene expression to predict treatment efficacy evaluating pretreatment and post-treatment in peripheral blood samples and pre- and post-treatment tumor tissue, expression of tumor specific oncoproteins including but not limited to ASPH, correlation of serum ASPH levels as determined by ELISA with bone marrow expression using flow cytometry, miRNA profiling to predict treatment efficacy using pretreatment and post-treatment peripheral blood samples and urine samples, cytokine and chemokine profiles in urine pretreatment and post-treatment and longitudinally throughout the trial, and assessment of the genetic landscape and changes in circulating tumor DNA pretherapy and post-therapy and correlation of the genetic landscape and changes in the circulating tumor DNA with clinical endpoints.

This phase 2, open-label, multi-center trial will evaluate the safety, immunogenicity and preliminary clinical efficacy of intradermally-delivered SNS-301 delivered using the 3M® hollow microstructured transdermal system (hMTS) device in patients with ASPH+ high risk MDS and CMML.

### Study Design

### Research Hypothesis

SNS-301 delivered by intradermal injection (ID) using the 3M® hollow microstructured transdermal system (hMTS) device will be generally safe, well tolerated, immunogenic and lead to anti-tumor activity in adult patients with ASPH-expressing high risk MDS and CMML.

### Overall Design

This phase 2, open-label, multi-center trial to evaluate the safety, immunogenicity and preliminary clinical efficacy of intradermally-delivered SNS-301 delivered using the 3M® hollow microstructured transdermal system (hMTS) device in patients with ASPH+ high risk MDS and CMML. Approximately 20 patients will be enrolled up to 15 institutions within the United States.

After consenting to participate in this clinical trial, participants will be screened for enrollment. The patient must have measurable ASPH expression in fresh bone marrow aspirate by flow cytometry conducted at a central laboratory. Ideally, patients should also provide an archival bone marrow sample from prior biopsy that is HMA treatment naive. On-treatment bone marrow aspirations/biopsies will be collected on day 42 (+/−3 days), then every 12 week and as indicated at the discretion of the investigator until documentation of response. For patients who respond and subsequently progress as determined per IWG criteria, a bone marrow aspirate/biopsy will be obtained at the time of disease progression at the discretion of the investigator. Enrollment may be allowed for patients unable to provide newly obtained bone marrow aspirate/biopsy with no intervening therapy or lacking requested archival, treatment-naïve samples after consultation with the Sponsor as long as there is an available sample to verify ASPH expression status.

### End of Study Definition

The clinical trial will be considered completed when all patients have had their three-year follow-up visit, death, lost to follow-up, withdrawal of consent, or when the Sponsor deems the study completed, whichever comes first.

### Study Population

Following informed consent, preliminary review of the inclusion/exclusion criteria and prior to any non-SOC procedures, bone marrow aspirate will be tested for the expression of ASPH. Patients who test positive for ASPH expression in fresh bone aspirate may continue screening in the study as per Table 10 and FIG. 23.

### Inclusion Criteria

The following paragraphs discuss the inclusion criteria.

(1) Each patient must provide signed IRB approved informed consent in accordance with institutional guidelines. (2) Each patient must be 18 years of age or older on the day of signing the informed consent, and able and willing to comply with all trial procedures. (3) Each patient must have a confirmed diagnosis of MDS or CMML excluding acute promyelocytic leukemia (APL, FAB M3). (4) High-risk-MDS/CMML status is evaluated: High-risk MDS is evaluated using IPSS-R criteria for categorization≥Intermediate Risk-3. High-risk CMML is evaluated using WHO criteria for CMML-2 characterized by peripheral blasts of 5% to 19%, and 10% to 19% bone marrow blasts and/or presence of Auer rods. (5) Each patient must be willing to provide a fresh bone marrow aspirate sample at pre-treatment and demonstrate ASPH expression by flow cytometry at a central laboratory. An archival sample (aspirate or biopsy) will be requested, if available. (6) Patients who have relapsed or are refractory/intolerant of hypomethylating agents (HMAs) or not responding to 4 treatment cycles of decitabine or 6 treatment cycles of azacytidine or progressing at any point after initiation of an HMA are eligible. Note: intolerance to azacitidine or decitabine defined as drug-related ≥Grade 3 liver or renal toxicity leading to discontinuation during past 2 years. (7) A patient that refuses or is not considered a candidate for intensive induction chemotherapy using consensus criteria for defining such patients. (8) Patients with CMML must have been treated with at least 1 prior therapy (hydroxyurea or an HMA). (9) A patient that has a performance status of 0 or 1 on Eastern Cooperative Oncology Group (ECOG) Performance Scale is included. (10) Each patient must have an ECG with no clinically significant findings conduction abnormalities or active ischemia as assessed by the investigator performed within 28 days prior to first dose. (11) Patients must demonstrate adequate organ function: renal, hepatic, coagulation parameters as defined below and obtained within 28 days prior to the first study treatment. Adequate end-organ function will be evaluated. Creatinine or calculated creatinine clearance will be calculated. Creatine clearance will be calculated per the Cockgroft-Gault formula. Creatinine should be ≤1.5 upper limit of normal (ULN) or ≥30 mL/min for a patient with a creatinine level >1.5× institutional ULN. Hepatic organ function will be assessed by measuring the total bilirubin, aspartate aminotransferase (AST) (serum glutamic oxaloacetic transaminase-SCOT) and alanine aminotransferase (ALT) (serum glutamic pyruvic transaminase-SGPT), and albumin levels. Total bilirubin should be ≤1.5×ULN or Direct bilirubin ≤ULN for patients with total bilirubin levels >1.5×ULN. AST and ALT should be ≤2.5×ULN. Albumin should be ≥3.0 g/dL. The international normalized ratio (INR) or prothrombin time (PT) and the activated partial thromboplastin time (aPTT) will be measured to assess a patient's coagulation. A patient's international normalized ratio (INR) or prothrombin time (PT) should be ≤1.5×ULN unless patient is receiving anticoagulant therapy as long as PT or partial prothrombin time (PTT) is within therapeutic range of intended use of anticoagulants. The activated partial thromboplastin time (aPTT) should be ≤1.5×ULN unless patient is receiving anticoagulant therapy as long as PT or PTT is within therapeutic range of intended use of anticoagulants. (12) Women of childbearing potential must agree to remain abstinent by refraining from heterosexual intercourse or using two highly effective contraceptive methods that result in a combined failure rate of <1% per year during the study course treatment period and for 180 days after the last dose of study drug. A woman is considered to be of childbearing potential if she is postmenarchal, has not reached a postmenopausal state (≥12 continuous months of amenorrhea with no identified cause other than menopause), and has not undergone surgical sterilization (removal of ovaries and/or uterus). Examples of contraceptive methods with a failure rate of <1% per year include bilateral tubal ligation, male sterilization, established, proper use of hormonal contraceptives that inhibit ovulation, hormone-releasing intrauterine devices, and copper intrauterine devices. The reliability of sexual abstinence should be evaluated in relation to the duration of the clinical trial and the preferred and usual lifestyle of the patient. Periodic abstinence (e.g., calendar, ovulation, sympto-thermal, or post-ovulation methods) and withdrawal are not acceptable methods of contraception. Male patients must agree that during the period specified above, men will not father a child. Male patients must remain abstinent (refrain from heterosexual intercourse with women of childbearing potential), must be surgically sterile (e.g., vasectomy) or use contraceptive methods that result in a failure rate of <1% per year during the treatment period and for at least 180 days after the last dose of study drug.

### Exclusion Criteria

The following paragraphs describe exclusion criteria. Patients who meet any of the following criteria will be excluded from trial entry. The sponsor will utilize both cancer-specific exclusion criteria and general medical exclusion criteria.

(1) A patient will be excluded if the patient has been administered any approved anti-cancer therapy including chemotherapy, targeted small molecule therapy or radiation therapy within 2 weeks prior to trial Day 0, or if the patient has not recovered (i.e., Less than or equal to grade 1 or returned to baseline level) from adverse events due to a previously administered agent; the following exceptions are allowed: hormone-replacement therapy or oral contraceptives and patients with grade 2 neuropathy or grade 2 alopecia. (2) Patients with evidence of rapid progression on prior therapy resulting in rapid clinical deterioration will be excluded from participation in the trial. (3) Patients that are currently participating and receiving trial therapy or has participated in a trial of an investigational agent within 28 days prior to Day 0 will be excluded. Patients who have entered the follow-up phase of an investigational trial may participate if it has been 28 days since the last dose of the previous investigational agent or device. (4) Patients with malignancies other than indications open for enrollment within 3 years prior to Day 0, are excluded with the exception of those patients that have a negligible risk of metastasis or death, those patients that have an expected curative outcome, and those patients undergoing active surveillance or treatment-naïve for indolent tumors. (5) Patients with a diagnosis of a core binding factor leukemia (t(8;21), t(16;16); or inv(16)) or diagnosis of acute promyelocytic leukemia (t(15;17)) will be excluded. (6) Patients that are pregnant or lactating or intending to become pregnant or father children within the projected duration of the trial starting with the screening visit through 180 months after the last dose of SNS-301 will be excluded. (7) Patients with an active or history of autoimmune disease or immune deficiency will be excluded. Non-limiting examples of autoimmune disease are Acute disseminated encephalomyelitis, Addison's disease, Ankylosing spondylitis, Antiphospholipid antibody syndrome, Aplastic anemia, Autoimmune hemolytic anemia, Autoimmune hepatitis, Autoimmune hypoparathyroidism, Autoimmune myocarditis, Autoimmune oophoritis, Autoimmune orchitis, Autoimmune thrombocytopenic purpura, Behcet's disease, Bullous pemphigold, Chronic inflammatory demyelinating polyneuropathy, Chung-Strauss syndrome, Crohn's disease, Dermatomyositis, Diabetes mellitus Type I, Dysautonomia, Epidermolysis bullosa acquista, Gestational pemphigold, Giant cell arteritis, glomerulonephritis, Goodpasture's syndrome, Granulomatosis with polyangiitis, Grave's disease, Guillain-Barré syndrome, Hashimoto's disease, IgA nephropathy, Inflammatory bowel disease, Interstitial cystitis, Kawasaki's disease, Lambert-Eaton myasthenia syndrome, Lupus erythematosus, Systemic Lupus erythematosus Lyme disease—chronic, Mooren's ulcer, Morphea, Multiple sclerosis, Myasthenia gravis, myositis, Neuromyotonia, Opsoclonus myoclonus syndrome, Optic neuritis, Ord's thyroiditis, Pemphigus, Pernicious anemia, Polyarteritis nodusa, Polyarthritis, Polyglandular autoimmune syndrome, Primary biliary cirrhosis, Psoriasis, Reiter's syndrome, Rheumatoid arthritis, Sarcoidosis, Scleroderma, Sjögren's syndrome, Takayasu's arteritis, Ulcerative colitis, vasculitis and Vogt-Kovanagi-Harada disease. Patients with a history of autoimmune-related hypothyroidism on a stable dose of thyroid replacement hormone as well as patients with adrenal insufficiency may be eligible for this trial. Patients with controlled Type I diabetes mellitus on a stable dose of insulin regimen may be eligible for this trial. (8) Patients with a history of HIV will be excluded from the trial. HIV antibody testing will be recommended per investigator's clinical suspicion. (9) Patients with active hepatitis B (hepatitis B surface antigen reactive) or active hepatitis C (HCV qualitative RNA detected) will be excluded. A patient will be tested for active hepatitis or hepatitis C per investigator's clinical suspicion. (10) Patients with severe infections within 4 weeks prior to enrollment, including, but not limited to, hospitalization for complications of infection, bacteremia, or the presence of any active infection requiring systemic therapy, will be excluded. (11) Patients that have received therapeutic oral or IV antibiotics within 2 weeks prior to Day 0 will be excluded. Patients receiving prophylactic antibiotics (e.g., for prevention of a urinary tract infection or to prevent chronic obstructive pulmonary disease exacerbation) are eligible. (12) Patients with a history or current evidence of any condition, therapy or laboratory abnormality that in the opinion of the treating investigator might confound the results of the trial or interfere with the subject's participation for the full duration of the trial will be excluded. Patients that have received a live, attenuated vaccine within 28 days prior to randomization or anticipation that such a live attenuated vaccine will be required during the trial will be excluded. (13) The influenza vaccination should be given during influenza season only (approximately October to March). Patients must not receive live, attenuated influenza vaccine (e.g., FluMist®) within 28 days prior to Day 0, during treatment, or within 90 days following the last dose of study treatment. (14) A patient with a known previous or ongoing, active psychiatric or substance abuse disorders that would interfere with cooperation with the requirements of the trial will be excluded. (15) A prisoner or patient who is compulsorily detained (involuntarily incarcerated) for treatment of either a psychiatric or physical (i.e. infectious disease) illness will be excluded. (16) A patient has been treated with systemic immunomodulating agents (including but not limited to IFNs, IL-2) within 6 weeks or five half-lives of the drug, whichever is shorter, prior to first dose, will be excluded. (17) A patient that has been treated with systemic immunosuppressive medication (including, but not limited to, corticosteroids, cyclophosphamide, azathioprine, methotrexate, thalidomide, and anti-TNF-α agents) within 2 weeks prior to initiation of study treatment, or anticipation of need for systemic immunosuppressive medication during the course of the study will be excluded unless the patient has received acute, low-dose (≤10 mg/day) systemic immunosuppressant medication or a one-time pulse dose of systemic immunosuppressant medication (e.g., 48 hours of corticosteroids for a contrast allergy), received inhaled, topical and intranasal steroids, or received mineral corticosteroids (e.g., fludrocortisone), corticosteroids for chronic obstructive pulmonary disease (COPD) for asthma, or low-dose corticosteroids for orthostatic hypotension or adrenal insufficiency.

### Screen Failures

Patients will be considered screen failures if they do not test positive for ASPH expression by flow cytometry in fresh bone marrow aspirate samples.

### Strategies for Recruitment and Retention

It is anticipated that up to 15 sites will be participate in the study in the United States. Patients will be recruited from either standalone outpatient clinics or hospital clinics.

### Study Treatment Administration: Description

The ASPH Nanoparticle Vaccine drug substance is a recombinant bacteriophage lambda construct that is engineered to display a fusion protein of phage gpD and a portion of the ASPH protein sequence. The HAAH-1λ (SNS-301) construct contains 199 amino acids from the N terminal region (amino acids 113-311) of the molecule.

The drug substance is characterized by testing that includes appearance, pH, and identity by dot blot using ASPH-specific monoclonal antibody, impurities (bioburden, endotoxin, host cell protein), determination of size distribution by particle analysis, quantitation by particle analysis, protein determination and potency by antigen enzyme-linked immunosorbent assay (ELISA). The drug product is a sterile, preservative-free solution.

### Study Treatment Administration: Dosing, Administration, and Preparation

The study treatment will be administered only to patients included in this study following the procedures set out in this clinical study protocol. Administration of the study treatment will be supervised by the Investigator or sub Investigator. Details of the exact time of administration of medication (day/month/year, hour:minute) will also be documented in the eCRF.

The vaccine will be delivered intra-dermally by a single-use 3M® hollow microstructured transdermal system (hMTS) device. Patients will be administered intradermally the SNS-301 dose of 1.0×10¹¹ particles in 1 mL per administration. Patients will receive SNS-301 on a staged schedule starting every three weeks for four doses, every six weeks for 6 doses and thereafter every twelve weeks for up to 24 months unless unacceptable toxicity.

For the first injection, the patient should be observed for 60 minutes. For subsequent injections, a 30-minute observation period is recommended after each study treatment.

Patients will receive their study treatment as described until disease progression, unacceptable toxicity, deemed intolerable by investigator or up to 24 months in patients without disease progression. There will be no dose reductions.

### Product Storage and Stability

SNS-301 will be stored at 2-8° C. Temperature excursions to <25° C. for less than 24 hours are acceptable. Storage at <25° C. for less than 24 hours is cumulative. Time spent at this temperature should be recorded in the drug accountability records.

The 3M® hollow microstructured transdermal system (hMTS) device should be maintained at room temperature.

### Randomization and Blinding

This is an open-label study, there will be no randomization or blinding.

All patients who sign the informed consent form (ICF) will be assigned a patient study number which will be retained for the duration of the study.

### Concomitant Therapy

All treatments including any prescription or over the counter medications taken by the patients seven days prior to screening and at any time during the study are regarded as concomitant treatments and must be documented in the appropriate section of the e-CRF. At subsequent visits, changes to current medications or medications used since the last documentation of medications will be recorded. Concomitant medications will be collected until 30 days after the last dose of study medication or until the start of a new anti-cancer treatment, whichever comes first.

The following concomitant treatments are permitted during the Phase 2 trial: supportive treatment as medically indicated, prophylactic antiemetic premedication including corticosteroids (low dose ≤10 mg/day prednisone equivalent) and 5 hydroxytryptamine 3 antagonists, and supportive treatment with cannabis if medically indicated.

The following medications are not permitted during the Phase 2 trial: concurrent treatment with other investigational drugs, concurrent treatment with any other anticancer therapy including radiotherapy, traditional herbal medicines because the ingredients of many herbal medicines are not fully studied, and their use may result in unanticipated drug-drug interactions that may cause or confound assessment of toxicity. However, if the investigator feels herbal medication is warranted the Sponsor should be consulted.

The initiation or increased dose of granulocyte colony-stimulating factors (e.g., granulocyte colony-stimulating factor, granulocyte/macrophage colony-stimulating factor, and/or pegfilgrastim) is strongly discouraged unless they are used per institutional policy in treating patients with MDS/CMML. Patients should be on a stable dose for at least six weeks prior to Day 0.

Patients are not allowed to receive immunostimulatory agents, including but not limited to IFN-α, IFN-γ, or IL-2, during the entire trial. These agents, in combination with study treatment, could potentially increase the risk for autoimmune conditions.

### Rescue Medication

Although most immune-mediated adverse events observed with immunomodulatory agents have been mild and self-limiting, such events should be recognized early and treated promptly to avoid potential major complications. Discontinuation of the study treatment may not have an immediate therapeutic effect due to the long half-life of the drug or longer drug effect, and there is no available antidote for the study treatment. In the unlikely event of an immune-mediated effect from a potent immune activation owing to SNS-301, management is recommended per investigator's discretion and/or institutional guidelines. In severe cases, immune-mediated toxicities may be acutely managed with topical corticosteroids, systemic corticosteroids, mycophenolate, or TNF-α inhibitors per investigator's discretion.

Patients should receive appropriate medical intervention necessary to treat medical conditions as they arise.

Dose Modification and/or Interruption

There will be no dose reductions for this study.

In case of an AE (grade 2 NCI-CTCAE V5.0 drug related AE), the dosing interval of 3 weeks can be extended to up 42 days to allow the recovery from a related toxicity and the subject will resume at the same dose. If the subject experiences the same grade or higher toxicity requiring a dose-delay at the subsequent cycle, the subject should be discontinued from study treatment.

Should there be a clinically significant AE or SAE recorded relating to a patient receiving anticoagulants, such as clinically noted bleeding, administration of SNS-301 will be held until the AE/SAE returns to baseline. Should there be two individual events of SNS-301 interruption for the same patient, then SNS-301 will be discontinued after consultation with the Medical Monitor and Study Sponsor.

### Study Intervention Discontinuation and Participant Discontinuation/Withdrawal

Discontinuation for the study treatment does not mean discontinuation from the study and the remaining study procedures should be completed as per the Time and Event Schedule. The patients may withdraw from study treatment if they decide to do so, at any time and irrespective of the reason. In addition, the Investigator or the Sponsor has the right to withdraw the patient from the study at any time. All efforts should be made to document the reason for discontinuation and this should be documented in the electronic case report form (eCRF).

Other criteria for possible discontinuation include disease progression, unacceptable toxicity as judged by the principal investigator, adverse events which are dose-limiting toxicities, withdrawal of consent, patient is lost to follow-up, patient non-compliance, use of another non-protocol anti-cancer treatment, and pregnancy.

Withdrawn patients will be followed according to the study procedures as specified in this protocol.

The patients may withdraw from the study follow-up period, before study completion if they decide to do so, at any time and irrespective of the reason. The reason for withdrawal from the study treatment or study follow-up will be documented in the eCRF. Patients may be replaced at the discretion of the Sponsor.

### Lost to Follow-Up

The Investigator should make every effort to re-contact the subject, to identify the reason why he/she failed to attend the visit, and to determine his/her health status, including at least his/her vital status. Attempts to contact such patients must be documented in the patient's records (e.g. times and dates of attempted telephone contact, receipt for sending a registered letter). It is suggested that the Investigator attempts to contact the patient three times before considering the patient lost to follow up.

### Study Assessment and Procedures

Table 10 shows an outline of the procedures required at each visit along with their associated windows. All patients must sign and date the most current approved ICF before any study specific procedures are performed. Procedures conducted as per standard of care or routine clinical management that are obtained before signing of the ICF may be utilized for screening/baseline purposes. All screening assessments may be performed within 28 days of Day 0 with the exception of screening labs (hematology and chemistry) which may be performed within 10 days of Day 0. Patients who discontinue will be asked to return to the clinic within 30 days of the last dose for a discontinuation visit. Generally, protocol waivers or exemptions will not be granted without discussion with the Sponsor.

### Efficacy Assessments: Bone Marrow Aspiration/Biopsy Assessment

Clinical responses will be assessed using 2006 International Working Group (IWG) response criteria for MDS and the international consortium proposal of uniform response criteria for myelodysplastic/myeloproliferative neoplasms (MDS/MPN) and CMML, published in 2015. Table 12 shows The Modified International Working Group response criteria for altering natural history of MDS (Cheson, et al. Blood, 15 Jul. 2006, volume 108, number 2).

TABLE 12
Tumor Assessment Criteria.

| Category | Response criteria (responses must last at least 4 wk) |
|---|---|
| Complete | Bone marrow: ≤5% myeloblasts with normal |
| remission | maturation of all cell lines* |
| | Persistent dysplasia will be noted*† |
| | Peripheral bloods‡ |
| | Hgb ≥ 11 g/dL |
| | Platelets ≥ 100 × 10⁹/L |
| | Neutrophils ≥ 1.0 × 10⁹/L† |
| | Blasts 0% |
| Partial remission | All CR criteria if abnormal before treatment except: |
| | Bone marrow blasts decreased by ≥50% over |
| | pretreatment but still >5% |
| | Cellularity and morphology not relevant |
| Marrow CR† | Bone marrow: ≤5% myeloblasts and decrease |
| | by ≥50% over pre-treatmentt |
| | Peripheral blood: if HI responses, they will be noted |
| | in addition to marrow CR† |
| Stable disease | Failure to achieve at least PR, but no evidence of |
| | progression for >8 wks |
| Failure | Death during treatment or disease progression |
| | characterized by worsening of cytopenias, |
| | increase in percentage of bone |
| | marrow blasts, or progression to a more |
| | advanced MDS FAB |
| | subtype than pretreatment |
| Relapse after | At least 1 of the following: |
| CR or PR | Return to pre-treatment bone marrow |
| | blast percentage |
| | Decrement of ≥50% from maximum remission/ |
| | response levels in granulocytes or platelets |
| | Reduction in Hgb concentration by ≥ 1.5 g/dL or |
| | transfusion dependence |
| Cytogenetic | Complete |
| response | Disappearance of the chromosomal abnormality |
| | without appearance of new ones |
| | Partial |
| | At least 50% reduction of the chromosomal |
| | abnormality |
| Disease | For patients with: |
| progression | Less than 5% blasts: ≥50% increase in blasts |
| | to > 5% blasts |
| | 5%-10% blasts: ≥50% increase to >10% blasts |
| | 10%-20% blasts: ≥50% increase to >20% blasts |
| | 20%-30% blasts: ≥50% increase to >30% blasts |
| | Any of the following: |
| | At least 50% decrement from maximum |
| | remission/response in granulocytes or platelets |
| | Reduction in Hgb by ≥2 g/dL |
| | Transfusion dependence |
| Survival | Endpoints (modified for SNS-301 study): |
| | Overall: death from any cause |
| | PFS: disease progression or death from MDS |

To convert hemoglobin from grams per deciliter to grams per liter, multiply grams per deciliter by 10.
MDS indicates myelodysplastic syndromes; Hgb, hemoglobin; CR, complete remission; HI, hematologic improvement; PR, partial remission; FAB, French-American-British; AML, acute myeloid leukemia; PFS, progression-free survival; DFS, disease-free survival.
*Dysplastic changes should consider the normal range of dysplastic changes.
†Modification to IWG response criteria.
‡In some circumstances, protocol therapy may require the initiation of further treatment (eg, consolidation, maintenance) before the 4-week period. Such patients can be included in the response category into which they fit at the time the therapy is started. Transient cytopenias during repeated chemotherapy courses should not be considered as interrupting durability of response, as long as they recover to the improved counts of the previous course.

Additionally, archival samples (aspirate or biopsy) will be requested, if available. After signing of the Informed Consent Form, bone marrow biopsy sample should be submitted in a timely manner. All patients will undergo a bone marrow aspirate at screening. Bone marrow aspirate/biopsy assessments (will be collected at 42 days (+3 days), Week 12 then after approx. 12 weeks until documentation of disease response or first evidence of disease progression if clinically feasible. Patients who are unable to undergo bone marrow aspirate/biopsy sample collection but otherwise meet criteria listed in the protocol may continue to receive study treatment. A bone marrow biopsy is acceptable with the exception of screening where a bone marrow aspirate is required for flow cytometry. For patients who respond and subsequently progress, an optional aspirate/biopsy may be obtained at the time of disease progression.

Fresh and archival tumor tissue samples should be representative tumor specimens in formalin-fixed paraffin embedded (FFPE) blocks (preferred) or at least 15 unstained slides, with an associated pathology report, should be submitted for intra-tumoral immunology assessments. Tissue slices of 4-5 microns are mounted on positively charged glass slides. Slides should be unbaked and stored cold or frozen.

For archival samples, the remaining tumor tissue block for all patients enrolled will be returned to the site upon request or 18 months after final closure of the trial database, whichever is sooner.

The remainder of samples obtained for trial-related procedures will be destroyed no later than 5 years after the end of the trial or earlier depending on local regulations. If the patient provides optional consent for storing samples for future research, the samples will be destroyed no later than 15 years after the date of final closure of the clinical database.

Peripheral blast counts will also be assessed as part of the efficacy measures. Peripheral blast counts that are done at the local laboratory will be collected on the eCRF.

### Safety Assessments: Demographics and Medical History

Demographics will include gender, year of birth, race and ethnicity. Medical history will include details regarding the patients overall medical and surgical history as well as detailed information regarding the subject's previous treatment, including systemic treatments, radiation and surgeries, pathology, risk stratification, immunophenotype, etc. since their original diagnosis. Progression data will be collected for all patients. Reproductive status and smoking/alcohol history will also be captured.

### Safety Assessments: Physical Examinations

A complete physical exam will include, at a minimum head, eyes, ears, nose, throat and cardiovascular, dermatological, musculoskeletal, respiratory, gastrointestinal and neurological systems. Height (screening only) and weight will also be collected. Additionally, any signs and symptoms, other than those associated with a definitive diagnosis, should be collected at baseline and during the study.

During the study, a targeted, symptom-directed exam, as clinically indicated will be performed within 72 hours of each dosing visit.

### Safety Assessments: Eastern Cooperative Oncology Performance Status

The health, activity and well-being of the patient will be measured by the ECOG performance status and will be assessed on a scale of 0 to 5 with 0 being fully active and 5 being dead. ECOG performance status will be collected within 72 hours of each dosing visit. Table 13 describes the ECOG Performance status scale.

TABLE 13
ECOG Performance Status
ECOG PERFORMANCE STATUS*

| GRADE | ECOG |
|---|---|
| 0 | Fully active, able to carry on all pre-disease |
| | performance without restriction |
| 1 | Restricted in physically strenuous activity but ambulatory |
| | and able to carry out work of a light or sedentary nature, |
| | e.g., light house work, office work |
| 2 | Ambulatory and capable of all self-care |
| | but unable to carry out any work activities. |
| | Up and about more than 50% of waking hours |
| 3 | Capable of only limited self-care, confined to |
| | bed or chair more than 50% of waking hours |
| 4 | Completely disabled. Cannot carry on any self-care. |
| | Totally confined to bed or chair |
| 5 | Dead |

*As published in: Am. J Clin. Oncol.: Oken, M.M. et al. Am J Clin Oncol 5:649-655, 1982.

### Safety Assessments: Vital Signs

Vital signs will include temperature, blood pressure, pulse rate and respiratory rate. For the first injection, the subject's vital signs should be determined within 60 minutes before the injection. Vital signs should be recorded at 60 (+5) minutes after the injection for the first injection. For subsequent injections, a 30-minute observation period is recommended. Patients will be informed about the possibility of delayed post-infusion symptoms and instructed to contact their trial physician if they develop such symptoms.

### Safety Assessments: Electrocardiograms

A 12-lead ECG will be obtained at screening and when clinically indicated. Patients should be resting in a supine position for at least 10 minutes prior to ECG collection.

### Safety Assessments: Clinical Safety Laboratory Assessments

Hematological toxicities will be assessed in term of hemoglobin value, white blood cell, neutrophil, platelet and, lymphocyte count according to NCI-CTCAE V5.0 AE grading.

Laboratory abnormalities (grade 1 and greater that are listed in the NCI-CTCAE V5.0) should be recorded on the AE page regardless of their causality. Laboratory abnormalities associated with a definitive diagnosis will not be recorded as and AE unless it has become worse since baseline. Test analytes include hematology analytes, such as hematocrit (Hct), hemoglobin (Hgb), platelet count, red blood cell (RBC) count, white blood cell (WBC) count, neutrophils, lymphocytes, eosinophils, monocytes, basophils, other cells, if any, platelets, and peripheral blast counts. Test analytes include coagulation analytes such as international normalized ratio (INR), activated partial thromboplastin time (aPTT), and other anticoagulant monitoring (if required). A HIV screen and/or hepatitis screen will be performed if suspected. Serum chemistry will measure albumin, alanine aminotransferase (ALT), aspartate aminotransferase (AST), alkaline phosphatase (ALP), blood urea nitrogen (BUN) or urea, bicarbonate or carbon dioxide (CO₂), creatinine, creatine phosphokinase (CPK), electrolytes (sodium, potassium, magnesium, chloride, calcium, phosphorous), glucose (either fasting or non-fasting), lactate dehydrogenase (LDH), total bilirubin (direct bilirubin if elevated), and total protein. Urinalysis will be performed to evaluate a patient's urine's specific gravity, pH, glucose, protein, ketones, and blood. A microscopic exam of the urine will be performed if abnormalities are found. A pregnancy test may be administered to confirm a patient is not pregnant. Table 10 describes the timing and frequency of these tests. Safety labs will be performed within 72 hours of each dosing visit.

### Hepatitis and HIV Screening

Patients should be tested for HIV locally prior to the inclusion into the trial if the investigator suspects HIV infection and HIV-positive patients will be excluded from the clinical trial. Hepatitis B surface antigen, anti-HBc antibody, anti-HBs antibody, and Hepatitis C antibody immunoassays should be collected per investigator's clinical suspicion during screening and tested locally. In patients who have positive serology for the anti-HBc antibody, HBV DNA should be collected prior to Day 0.

### Pregnancy Test

A Serum pregnancy test (for women of childbearing potential, including women who have had a tubal ligation) must be performed and documented as negative within 72 hours prior to each dose.

### Urinalysis

Urinalysis includes specific gravity, pH, glucose, protein, ketones, blood, and a microscopic exam if abnormal results are noted.

### Creatine Phosphokinase (CPK)

CPK will be performed at screening and at the discontinuation visit.

### Immunogenicity Assessments: Urine

Urine samples will be obtained for biomarker evaluation. Samples may be tested for the presence and level of various cytokines by ELISA which may be indicative of activated immune responses. Samples may also be tested by ELISA for the presence and level of ASPH and/or other cancer biomarkers which may be indicative of cancer status. Samples may also be processed to obtain tumor cells (and their derivatives) for further determination and analysis of cancer status. miRNA profiling of pre and post-treatment urine samples may also be performed to predict treatment efficacy.

### Immunogenicity Assessments: Blood Assays

Blood assays include those measured in serum, plasma and whole blood/PBMCs.

### Immunogenicity Assessments: Serum and Plasma

Serum and plasma are collected for the direct measure of ASPH levels, anti-ASPH antibodies, anti-phage antibodies and other tumor biomarkers.

### Immunogenicity Assessments: ASPH

Subject sera and/or plasma will be tested for the presence of ASPH and/or exosomes that contain ASPH on their surface by ELISA using several different monoclonal antibodies that are reactive with the ASPH protein. The presence of ASPH in serum or plasma is an indicator of cancer status. Alterations in ASPH levels may be indicative of response to treatment.

### Immunogenicity Assessments: Anti-ASPH Antibodies

Production of anti-ASPH antibodies is a direct result of an active immune response to the vaccine. Levels of anti-ASPH antibody are expected to rise during an active immune response and should reach a plateau level at maximal response. Continued and regular boosting of the vaccine during the course of treatment is expected to maintain or restore this level of anti-ASPH antibody in serum.

### Immunogenicity Assessments: Anti-Phage Antibodies

Because the vaccine is delivered using a bacteriophage vector, production of anti-phage antibodies is also expected and is a direct result of an active immune response to the vaccine. High levels of anti-phage antibody may result in neutralization of further doses/boosts of vaccine. During the Phase 1 clinical study it was found that the use of a lower dose of vaccine during initial vaccination attenuated the production of anti-phage antibodies relative to anti-ASPH antibodies and this finding contributed to the selection of the dose for the current trial. Levels of anti-phage antibodies will be monitored here to ascertain if any correlation exists between the production of anti-phage antibodies and reduced efficacy of the vaccine.

### Immunogenicity Assessments: Other Tumor and Immune Biomarkers

Levels of other cancer biomarkers and cytokines may also be tested in serum and/or plasma and may also be used to monitor cancer status and response to treatment. miRNA profiling of pre and post-treatment serum and/or plasma samples may also be performed to predict treatment efficacy.

Immunogenicity Assessments: Whole Blood/Peripheral Blood Mononuclear Cells (PBMCs) and/or Bone Marrow Aspirates (BMMCs)

PBMCs are collected to monitor overall and specific immune responses.

### Immunophenotyping

Immunophenotyping will be performed by flow cytometry to monitor the levels of all immune cells including B-cells, CD4+ T-cells, CD8+ T-cells, NK cells, monocytes, neutrophils, eosinophils and myeloid derived suppressor cells (MDSCs). In patients mounting an active immune response it is expected for the percentages of certain cell types to increase.

Gene transcript signatures from PBMCs to assess the profile of immune-related gene transcripts may be performed on PBMCs with or without prior in vitro stimulation.

### B-Cells

B-cells form the humoral (antibody) response arm of the immune system. Vaccination with SNS-301 is expected to result in maturation of anti-ASPH specific B-cells.

### B-Cell Profiling

As B-cells mature they transition through multiple stages that are distinguishable by the analysis of the presence or absence of specific surface antigens. Percentages of naïve B-cells, transitional B-cells, activated B-cells, plasmablasts, plasma cells and memory B-cells will be determined by multi-parameter flow cytometry.

### ASPH-Specific B-Cells

ASPH-specific B-cells are a direct measure of the immune response to the SNS-301 vaccine. Flow cytometry will be used to determine the changes in the levels of ASPH-specific B-cells. Furthermore, these B-cells may be isolated, cloned and expanded ex vivo and the resulting anti-ASPH antibodies characterized via epitope mapping.

### T-Cells

T-cells form the cellular arm of the immune response. Vaccination with SNS-301 is expected to result in maturation and activation of ASPH specific T-cells.

### T-Cell Profiling

The cellular immune response can generally be characterized as having two primary arms, CD4+ helper T-cell responses and CD8+ cytotoxic T-cell responses. In preclinical studies as well as the phase 1 clinical trial of SNS-301, activation of both T-cell subsets was noted. Furthermore, immune responses are often hampered by the presence of regulatory T-cells which may downregulate T-cell responses. Multi-parameter flow cytometry will be used to characterize the various subsets of T-cells in peripheral blood during the entire course of the study. Flow cytometric assays will also be utilized to assess the presence of cells that are known to play a role in immune suppression and may include an examination of the influence of these cells on the induction or expansion of an immune response after immunotherapy. Markers that may be used for this purpose include CD3, CD16, CD19, CD20, CD56, CD1 b, CD14, CD15, CD33 and HLA-DR.

### ASPH-Specific T Cells

T cell responses will be assessed using antigen-specific IFN-γ ELISpot assay using antigen presenting cells loaded with either full-length recombinant ASPH protein or overlapping peptide libraries covering the SNS-301 antigens. Antigen specific T cell responses will also be assessed via flow cytometry. Flow cytometric assays may include an examination of the influence of immunotherapy on the ability of patient T cells to exhibit phenotypic markers associated with cytolytic potential, activation or exhaustion after stimulation by peptides corresponding to SNS-301 antigens. Markers that may be used for this purpose include CD3, CD4, CD8, CD137, CD69, CD38, PD 1, Granzyme A, Granzyme B and Perforin. These markers may change relative to new data becoming available that is informative for this assessment. Additionally, T-cell responses to general immune stimulators may be evaluated in order to track general cellular immune competence during the trial.

Additionally, ASPH-specific T-cells may be isolated, cloned and expanded ex vivo. For expansion antigen presenting cells loaded with either full-length recombinant ASPH protein or overlapping peptide libraries covering the SNS-301 antigens would be employed. These T-cells may be characterized by sequencing of their T-cell receptors (TCRs) to assess diversity and putative antigen specificity.

### Tissue

Tissue will be collected as described in the Bone Marrow Aspiration/Biopsy Assessment section herein.

### Tissue Assays

Available tumor tissue collected from pre- and post-treatment may be assessed for the presence of immune cells using immunohistochemistry or immunofluorescence. The presence of immune signatures may also be analyzed through the assessment of various transcripts suggestive of an inflammatory or an immunosuppressive tissue microenvironment.

Tumor tissue will be collected for immunology assessments including but not limited to markers related to inflammation, suppression, T cell infiltration, and associated tumor microenvironment characteristics. Tumor infiltrating lymphocytes may be isolated and subjected to single cell expression profiling and/or TCR sequencing. In addition, exploratory biomarkers may be evaluated.

### ASPH Flow Cytometry Test

ASPH expression (positive or negative) in bone marrow aspirate as assessed by flow cytometry for enrollment is determined based on a cut-off of ≥20% ASPH positive blasts out of total blasts. ASPH positive blasts out of total blasts. Bone marrow aspirates or peripheral blood is collected from the patient and mononuclear cells are isolated by density gradient centrifugation. Cells are stained with ClearLLab M reagents (Beckman Coulter, Cat # B66812, DEN160047) as well as an antibody specific for ASPH and read on a Navios EX flow cytometer (Beckman Coulter, K162897). The ClearLLab M reagents include antibodies specific to the following cell surface markers and labelled with the indicated fluorophores, CD7-FITC/CD13-PE/CD34-ECD/CD33-PC5.5/CD45-PC7. The anti-ASPH antibody is labelled with alexa 647. A gating strategy is used to identify blasts by selecting the CD45dim, SSClow population and then selecting the CD33+, CD34+ population. This population of cells is taken as total blasts. The percentage of total blasts that stain with anti-ASPH (MFI≥10) are subsequently determined. The cut-off of ≥20% ASPH positive blasts out of total blasts was determined based on preliminary studies of a panel of patient-derived bone marrow aspirates from individuals with a known diagnosis of MDS. The percentage of ASPH+ blasts out of the total blast population is determined however, for eligibility the ASPH expression will be expressed as positive or negative. Testing for ASPH via flow cytometry will be conducted in a central laboratory.

Analysis of ASPH via peripheral blood (PBMCs) will be conducted in the same manner throughout the study

### ASPH Immunohistochemistry

Tissues are supplied as formalin-fixed paraffin embedded (FFPE) blocks. Tissue slices of 4-5 microns are mounted on positively charged glass slides. Tissue is deparaffinized and rehydrated, quenched with hydrogen peroxide and blocked with horse serum. Slides are stained overnight at 4° C. with an ASPH-specific murine monoclonal or a non-relevant mouse IgG as a negative control. Detection employs a secondary anti-mouse antibody and a chromogenic substrate. Slides are counterstained with hematoxylin and cover slipped. Semiquantitative analysis of staining intensity and distribution of ASPH levels is evaluated according to the following scale (0, negative; 1+, moderate; 2+, strong; and 3+, very strong immunoreactivity).

### Future Biomedical Research

The following samples are obtained as part of the study, if any leftover samples remain, they may be used for future biomedical research either during the course of the study or after the study has completed. The samples include: leftover tumor tissue, leftover RNA or DNA isolated from biological samples (blood, urine, tumor), and leftover biomarker samples (serum, plasma and PMBCs)

### Concomitant Medications

Concomitant medications include any prescription medications or over-the-counter medications. At screening, any medications the patient has used within the 7 days prior to the screening visit should be documented. At subsequent visits, changes to current medications or medications used since the last documentation of medications will be recorded.

Patients who are receiving anticoagulants will have anticoagulant specific drug level and/or anticoagulant specific factor Xα levels obtained at baseline, at each administration of SNS-301, and at the end of study visit to ensure that these levels remain within therapeutic range throughout the duration of the trial. In the event of clinically noted bleeding, these tests will be obtained at the time of bleeding as well. Investigators should use tests routinely used in clinical practice to monitor patients receiving Warfarin, Heparin and/or Low Molecular Weight Heparins, along with the monitoring schedule provided above. Should there be two individual events of SNS-301 interruption for the same patients, then SNS-301 will be discontinued after consultation with the Medical Monitor and Study Sponsor. Clinical management and further workup of the coagulation pathway disturbance will be at the discretion of the investigator. The following medications are not permitted during the Phase 2 trial: concurrent treatment with other investigational drugs, concurrent treatment with any other anticancer therapy including radiotherapy, traditional herbal medicines because the ingredients of many herbal medicines are not fully studied, and their use may result in unanticipated drug-drug interactions that may cause or confound assessment of toxicity. However, if the investigator feels herbal medication is warranted the Sponsor should be consulted.

The initiation or increased dose of granulocyte colony-stimulating factors (e.g., granulocyte colony-stimulating factor, granulocyte/macrophage colony-stimulating factor, and/or pegfilgrastim) is strongly discouraged unless they are used per institutional policy in treating patients with MDS/CMML. Patients should be on a stable dose for at least six weeks prior to Day 0.

Patients are not allowed to receive immunostimulatory agents, including but not limited to IFN-α, IFN-γ, or IL-2, during the entire trial. These agents, in combination with study treatment, could potentially increase the risk for autoimmune conditions

### Follow Up

Survival follow-up information will be collected via telephone calls, patient medical records, and/or clinical visits approximately every 3 months for up to 3 years, until death, lost to follow-up, withdrawal of consent, trial termination by Sponsor. All patients will be followed for survival and new anticancer therapy information unless the patient requests to be withdrawn from follow-up; this request must be documented in the source documents and signed by the investigator. If the patient discontinues study treatment without documented clinical disease progression, every effort should be made to follow up regarding survival, progression (if not already progressed), and new anti-cancer therapy.

### Adverse Events

AEs will be collected from the time of informed consent until 30 days after the last dose of study treatment or until initiation of another anti-cancer therapy, whichever occurs first. SAEs and AESIs will be collected from the time of informed consent until 90 days after the last dose of study treatment of until initiation of anti-cancer therapy, whichever occurs first. See Section 9.3 for additional details on Adverse Events and Serious Adverse Events.

### Definition of Adverse Event

Adverse event is defined as any untoward medical occurrence associated with the use of a drug in humans, whether or not considered drug related and occurs after the patient is given the first dose of study drug. Any AE that occurs prior to the first dose is part of the medical history.

Abnormal laboratory values should not be listed as separate AEs if they are considered to be part of the clinical syndrome that is being reported as an AE unless worsened on study treatment. It is the responsibility of the Investigator to review all laboratory findings in all patients and determine if they constitute an AE. Medical and scientific judgment should be exercised in deciding whether an isolated laboratory abnormality should be classified as an AE. Any laboratory abnormality (grade 1 and greater that are listed in the NCI-CTCAE V5.0 considered to constitute an AE should be reported on the Adverse Event CRF.

Pre-planned procedures (surgeries or therapies) that were scheduled prior to the start of study drug exposure are not considered AEs. However, if a pre-planned procedure is performed earlier than anticipated (e.g., as an emergency) due to a worsening of the pre-existing condition, the worsening of the condition should be captured as an AE.

Progression of the cancer under trial is not considered an adverse event unless it is considered to be drug related by the investigator. Patients will be encouraged to spontaneously report any AE.

Patients will be encouraged to spontaneously report any AE. Patients will be questioned and/or examined by the Investigator and his/her medically qualified designee for evidence of AEs. The questioning of study patients with regard to the possible occurrence of AEs will be generalized, such as, “How have you been feeling since your last visit?” Information gathering for AEs should generally not begin with direct solicitation from patients regarding the presence or absence of specific AEs. Study personnel will ask open ended questions to obtain information about AEs at every visit. Date and time of onset and resolution (if applicable) of the AE will be documented in the patient's clinical notes.

A suspected adverse reaction means any AE for which there is a “reasonable possibility” that the drug caused the AE. For the purpose of reporting under this protocol, “reasonable possibility” means there is evidence to suggest a causal relationship between the drug and the AE.

An AE is considered unexpected if the AE is not listed in the current IB or is not listed in the IB at the specificity or severity observed.

### Definition of Serious Adverse Events

A serious adverse event (SAE) is an AE that: is fatal, is life-threatening, meaning the patient was, in the view of the Investigator, at immediate risk of death from the reaction as it occurred, e.g., it does not include a reaction that, had it occurred in a more serious form or progressed, might have caused death, is a persistent or significant disability or incapacity or substantial disruption of the ability to conduct normal life functions, requires or prolongs inpatient hospitalization, is a congenital anomaly or birth defect. Other important medical events may be considered SAEs when, based upon appropriate medical judgment, they may jeopardize the patient and may require medical or surgical intervention to prevent one of the outcomes as listed above in this definition. Examples of such medical events include allergic bronchospasm requiring intensive treatment in an emergency room or at home, blood dyscrasias or convulsions that do not result in inpatient hospitalization, or the development of drug dependency or drug abuse

The Medical Monitor will advise the Investigator regarding the nature of any further information or documentation that is required. The Investigator should provide the following documentation at the time of notification if available: a SAE Form, a AE (CRF) page, concomitant and support medication pages, relevant diagnostic reports, relevant laboratory reports, and admission notes and hospital discharge summary (when available).

### Classification of Serious Adverse Events (SAEs)

Death in itself is not an AE. Death is an outcome of an AE. Progression of the cancer under trial is not considered an adverse event unless it is considered to be drug related by the investigator. The patient may not have been receiving an investigational medicinal product at the occurrence of the event. Dosing may have been given as treatment cycles or interrupted temporarily before the onset of the SAE but may have contributed to the event. Complications that occur during hospitalizations are AEs. If a complication prolongs the hospitalization, it is an SAE. Inpatient hospitalization means that the patient has been formally admitted to a hospital for medical reasons, for any length of time. This may or may not be overnight. It does not include presentation and care within an emergency department nor does it include full day or overnight stays in observation status.

The following hospitalization scenarios are not considered to meet the criteria for a serious adverse event: hospitalization for respite care, hospitalization to perform an efficacy measurement for the trial and hospitalization for an elective surgery for a pre-existing condition.

The Investigator will attempt to establish a diagnosis of the event on the basis of signs, symptoms, and/or other clinical information. In such cases, the diagnosis will be documented as the AE and/or SAE and not the individual signs/symptoms.

### Classification of an Adverse Event: Severity

Adverse events will be graded by the Investigator using the NCI-CTCAE 5.0 graded 1-5. Grade refers to the severity of the AE. For events not described in the NCI CTCAE, the Investigator will assign grades as 1=mild, 2=moderate, 3=severe, 4=life-threatening, and 5=fatal based on this general guideline: Grade 1: Mild; asymptomatic or mild symptoms; clinical or diagnostic observations only; intervention not indicated; Grade 2: Moderate; minimal, local or noninvasive intervention indicated; limiting age-appropriate instrumental ADL; Grade 3: Severe or medically significant but not immediately life-threatening; hospitalization or prolongation of hospitalization indicated; disabling; limiting self-care ADL; Grade 4: Life-threatening consequences; urgent intervention indicated; Grade 5: Death related to AE. (Grade 5 (Death) may not appropriate for some AEs and therefore may not be an option.) The highest level of severity attained for each AE will be recorded in the CRFs. An instrumental activity of daily living (ADL) refer to preparing meals, shopping for groceries or clothes, using the telephone, managing money, etc. A self-care ADL refers to bathing, dressing and undressing, feeding self, using the toilet, taking medications, and not bedridden.

### Determination of Relationship to Study Intervention

Events will be considered treatment related if classified by the Investigator as possible related, probable related, or related associated with the use of the drug. Association of events to the study treatment will be made using the following definitions:

The assessment of relationship of AEs to the administration of study drug is a clinical decision based on all available information at the time of the completion of the CRF. The following categories will be used to define the causality of the AE. The highest level of relatedness attained for each AE will be recorded in the CRFs.

The category “Not Related” is applicable to those AEs that are clearly due to extraneous causes (concurrent drugs, environment, etc.) and do not meet the criteria for drug relationship listed under Unlikely Related; Possibly; Probably; and Related.

An AEs that is judged to be unlikely related to the study drug administration is called “Unlikely Related”. An AE may be considered to be Unlikely Related when it meets at least two (2) of the following criteria: the AE does not follow a reasonable temporal sequence from administration of the study drug, the AE could readily have been produced by the patient's clinical state, environmental or toxic factors, or other modes of therapy administered to the patient, the AE does not follow a known or expected response pattern to the study drug, the AE does not reappear or worsen when the study drug is re-administered.

An AE that is judged to be perhaps related to the study drug administration is called “Possibly Related.” An AE may be considered possibly related when the AE meets at least one of the following criteria: the AE follows a reasonable temporal sequence from administration of the study drug; the AE could not readily have been produced by the patient's clinical state, environmental or toxic factors, or other modes of therapy administered to the patient; or the AE follows a known or expected response pattern to the study drug.

An AE that is felt with a high degree of certainty to be related to the study drug administration is “Probably Related.” An AE is considered Probably Related when it meets at least two of the following criteria: the AE follows a reasonable temporal sequence from administration of the study drug; the AE could not be reasonably explained by the known characteristics of the patient's clinical state, environmental or toxic factors, or other modes of therapy administered to the patient; the AE disappears or decreases on cessation or reduction in study drug dose; and the AE follows a known or expected response pattern to the study drug. There are exceptions when an AE does not disappear upon discontinuation of the drug, yet drug relatedness clearly exists (e.g., bone marrow depression, fixed drug eruptions, tardive dyskinesia, etc.).

An AE that is incontrovertibly related to study drug administration is “Related.” An AE may be assigned to this category if it meets at least the first three of the following criteria: (i) the AE follows a reasonable temporal sequence from administration of the study drug, (ii) the AE could not be reasonably explained by the known characteristics of the patient's clinical state, environmental or toxic factors, or other modes of therapy administered to the patient, (iii), It disappears or decreases on cessation or reduction in study drug dose. There are exceptions when an AE does not disappear upon discontinuation of the drug, yet drug relatedness clearly exists (e.g., bone marrow depression, fixed drug eruptions, tardive dyskinesia, etc.), (iv) the AE follows a known or expected response pattern to the study drug, (v) the AE reappears or worsens when the study drug is re-administered.

Any event deemed possibly related, probably related or related will be reported as a treatment emergent adverse event.

### Serious and Unexpected Suspected Adverse Reactions

The Sponsor must report any suspected adverse reaction that is both serious and unexpected. The Sponsor must report an adverse event as a suspected adverse reaction only if there is evidence to suggest a causal relationship between the drug and the adverse event, such as:

1. A single occurrence of an event that is uncommon and known to be strongly associated with drug exposure (e.g., angioedema, hepatic injury, Stevens-Johnson Syndrome);

2. One or more occurrences of an event that is not commonly associated with drug exposure, but is otherwise uncommon in the population exposed to the drug (e.g., tendon rupture);

An aggregate analysis of specific events observed in a clinical trial (such as known consequences of the underlying disease or condition under investigation or other events that commonly occur in the study population independent of drug therapy) that indicates those events occur more frequently in the drug treatment group than in a concurrent or historical control group.

Reports will be made as soon as possible, and in no event later than seven (7) calendar days if the event is a death or is life threatening and 15 calendar days for all other reportable events after the Sponsor's initial receipt of the information. Each written notification may be submitted on a CIOMS-I form, a FDA Form 3500A, or in a tabular or narrative format in accordance with regulatory requirements. In each report, the Sponsor will identify all safety reports previously filed concerning a similar suspected adverse reaction and will analyze the significance of the suspected adverse reaction in light of the previous, similar reports.

Follow-up information to a safety report will be submitted as soon as the relevant information is available. If the results of a Sponsor's investigation show that an AE not initially determined to be reportable is, in fact, reportable, the Sponsor will report the suspected AE in a written safety report as soon as possible, but in no event later than 15 calendar days after the determination is made. Results of investigations of other safety information will be submitted, as appropriate, in an information amendment or annual report.

If an investigator receives an IND safety report or other specific safety information (e.g., SUSAR, summary or listing of SAEs) from the sponsor, the investigator will review and file along with the Investigator's Brochure and will notify the IRB/IEC, if appropriate according to local regulations. In these instances, the ICF may need to be revised to inform the patient of any new safety concern.

### Unanticipated (Serious) Adverse Device Effect (UADE)

A UADE is any serious adverse effect on health or safety or any life-threatening problem or death caused by, or associated with, a device, if that effect, problem, or death was not previously identified in nature, severity, or degree of incidence in the investigational plan or application (including a supplementary plan or application), or any other unanticipated serious problem associated with a device that relates to the rights, safety, or welfare of patients.

Per the definition above, a UADE is a type of SAE that requires expedited reporting on the part of the Sponsor. As a reminder, all SAEs regardless of relationship to device, drug or procedure are to be reported to Sponsor by the trial Investigator within 24 hours. Sponsor will assess each device related SAE to determine if anticipated based on prior identification within the investigational plan. The Sponsor may notify a regulatory authority within the time frame specified by local requirements but no later than 10 business days for UADE.

### Stopping Rules

Stopping rules for adverse events will be employed for this trial. The trial will be stopped if any adverse experience of any related death, grade 4 autoimmune toxicity or any grade 4 toxicity that is furthermore considered possibly, probably or definitely related to study drug should occur. Any related death, grade 4 autoimmune toxicity and any grade 4 toxicity that is furthermore considered to be possibly, probably or definitely related to study drug will be submitted will be submitted to regulatory agencies within the expedited safety reporting criteria.

### Adverse Events of Special Interest

Adverse events that occur during or within 24 hours after study treatment administration and are judged to be related to study treatment infusion should be captured as a diagnosis (e.g., “infusion-related reaction”) on the Adverse Event eCRF. If possible, avoid ambiguous terms such as “systemic reaction.” If a patient experiences both a local and systemic reaction to the same dose of study treatment, each reaction should be recorded separately on the Adverse Event eCRF.

Administration site reaction will be considered an adverse event of special interest (AESI). The area around the administration site will be assessed by a medically qualified individual for adverse reactions at least 30 minutes post study drug administration. The Investigator will grade any ASRs according to the NCI-CTCAE V5.0 (excluding the actual expected micro-injection punctures).

Patients will be required to report any change in the administration site and return to the clinic for evaluation by the Investigator.

### Guidance for Investigators

Based on the preclinical data and the role of ASPH in post-translational modification of proteins involved in the clotting and anticoagulant pathways (Factors VII, IX, X, Protein C), there may a potential for abnormal coagulation with SNS-301. Should there be a clinically significant AE or SAE recorded, such as clinically noted bleeding, administration of SNS-301 will be held until the AE/SAE returns to baseline. Should there be two individual events of SNS-301 interruption for the same patient, then SNS-301 will be discontinued after consultation with the Medical Monitor and Sponsor. Clinical management and further workup of the coagulation pathway disturbance will be at the discretion of the treating physician.

### Reporting of Pregnancy

If pregnancy occurs in a female subject, or female partner of a male patient while the patient is on treatment or until six months after the last dose, the sponsor will be notified within 24 hours of learning of the pregnancy. The pregnancy will be followed until birth or termination. Abnormal pregnancy outcomes (e.g., spontaneous abortion, fetal death, stillborn, congenital anomalies, ectopic pregnancy) are considered SAEs.

### Time Period and Frequency for Event Assessment and Follow Up

All AESIs and SAEs, including death due to any cause, that occur during this study and until 90 days after the last dose of study treatment or until the start of a new anti-cancer treatment, whichever comes first, whether or not expected and regardless of causality, must be reported to the Medical Monitor immediately upon discovery of the event, using an SAE Form.

All AEs will be collected from the time of signing the informed consent form until 30 days after the last dose after the last dose of study treatment or until the start of a new anti-cancer treatment, whichever comes first.

Any medical condition that begins before the start of study intervention but after obtaining informed consent will be recorded on the Medical History section of the case report form not the AE section. However, if the patient's condition worsens during the study, the event will be recorded as an AE.

All AEs/SAEs will be captured on the appropriate case report form. Information to be collected includes event description, date of onset, severity, relationship to study intervention and date of resolution.

### Statistical Considerations

Sample Size Determination: Approximately 20 patients with ASPH+ high risk MDS and CMML (≤5/20 patients) will be enrolled. The sample size for this study is in alignment with other oncology studies with objectives of assessing safety and tolerability and initial estimates of the antitumor activity rather than on statistical power calculations

### Populations for Analysis

The following analysis populations will be used for presentation of the data: Safety Population: The safety analysis will be based on the Safety Population, which comprises all patients who receive at least 1 dose of the study treatment; Per Protocol Population: All patients who receive at least 1 dose of the study treatment and have at least one post baseline efficacy response assessment per the IWG without any protocol deviation(s) that would compromise the effectiveness of the treatment will be analyzed in the Per Protocol Population. Subjects who discontinue the study after at least one post baseline efficacy assessment due to disease clinical progression will be included; Immunology Population: All patients who receive at least 1 dose of the study treatment and have at least one valid post baseline immunologic assessment available will be analyzed in the Immunology Population.

### Statistical Analyses

A detailed methodology for summaries and displays of the data collected in this study will be documented in a Statistical Analysis Plan (SAP) that will be finalized prior to database lock. The study analyses will not include any formal statistical testing. All analyses will be considered descriptive and exploratory.

### General Methods

For continuous variables, descriptive statistics (number (n), mean, median, standard deviation, minimum and maximum) will be presented. For categorical variables, frequencies and percentages will be presented. For time-to-event variables, percentages of patients experiencing that event will be presented and median time-to-event will be estimated using the Kaplan-Meier method. As appropriate, a 95% CI will be presented. Graphical displays will be presented, as appropriate.

Subjects demographic characteristics including age, gender, and race will be analyzed, with categorical variables summarized in frequency tables while continuous variables summarized using mean (standard deviation) and median (range).

All data collected will be presented in by-patient data listings.

### Efficacy Analyses

The primary efficacy analysis will be performed on the ITT/Safety population. The PP population will be the subset of the Safety Population that is compliant with the protocol and excludes subjects with major protocol violations and have at least 1 post baseline efficacy response assessment per IWG 2006 criteria. The protocol violation criteria will be defined in the SAP. Analyses of efficacy variables will be performed on subgroups of interest (MDS & CMML) and will be outlined in the SAP.

ORR is defined as the proportion of patients with a confirmed best response of CR or PR by IWG. Overall response rate will be estimated, and 95% CI based on the exact binomial distribution will be presented, including number and percent of patients in each overall response category.

The primary analysis will be based on the objective response rate (CR+PR). An additional analysis of ORR will be performed based on the best overall response (BOR) during the study.

DOR, or the time from date of first response to date of progression, where patients without progression are censored at date of last valid disease assessment, will be calculated. DCR, or the proportion of patients with SD or better (CR+PR+SD) will be calculated. PFS, or the time from date of start of treatment to date of progression, where patients without progression are censored at date of last valid disease assessment, will be calculated. OS, or the time from date of start of treatment to date of death or censored at date of last contact, will be calculated.

Marrow CR and cytogenic responses will be analyzed separately.

### Safety Analyses

The safety analysis will be based on the Intent-to-Treat (ITT)/Safety Population, which comprises all participants who receive at least 1 dose of the study treatment.

Safety evaluations will be based on the incidence, severity, attribution and type of AEs, and changes in the patient's vital signs, and clinical laboratory results, analyzed using the safety analysis set.

Summarization of toxicity data will focus on incidence of treatment-emergent adverse events. Treatment-emergent adverse events are defined as any AE that occurs during or after administration of the first dose of treatment through 30 days after the last dose, any event that is considered study drug-related regardless of the start date of the event, or any event that is present at baseline but worsens in intensity. The incidence of serious adverse events, adverse events, drug-related adverse events, and adverse events leading to discontinuation or death will be presented in tabular form by system organ class and preferred term. Adverse events will be assessed for severity according to the NCI CTCAE, version 5.0.

Other safety evaluations including vital sign, laboratory and physical exam results will be presented over time.

### Other Analyses

The Immunology Population will be used to assess immune response. Antigen-specific cellular immune response assessed by but not limited to Interferon-γ secreting T lymphocytes will be summarized by visit. Immune related gene expression will be evaluated with pre- and post-treatment tissue biopsies. Cytokine and chemokine profiles will be summarized by visit.

Additional exploratory analyses may be performed, including evaluation of relationship between efficacy endpoints and immunology parameters.

### Pharmacodynamics

Exploratory pharmacodynamic (PD) analysis will be performed using dose, vaccine-specific antibody response (geometric mean titer), antigen-specific T and B cell indices, and the relative expression of ASPH in each subject's tumor. The PD will be balanced and optimized to the degree of antigen-specific immune response and minimized for the production of regulatory immune processes.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

1. A method of purifying and concentrating a bacterial lysate comprising a lambda-phage expressing a cancer antigen or a fragment thereof to produce a nanoparticle vaccine, the method comprising:
i) performing tangential flow filtration (TFF) on the bacterial lysate comprising a lambda-phage expressing a cancer antigen or a fragment thereof to produce a concentrated bacterial lysate;
ii) adding 100% ethanol to the concentrated bacterial lysate to produce a bacterial lysate and ethanol mixture having an about 25% ethanol concentration;
iii) performing TFF on the bacterial lysate and ethanol mixture to produce a concentrated ethanol-treated bacterial lysate;
iv) diluting the ethanol-treated bacterial lysate and treating the ethanol-treated bacterial lysate with ultraviolet (UV) light to produce a UV-treated, ethanol-treated bacterial lysate;
v) performing TFF on the UV-treated, ethanol-treated bacterial lysate to produce a nanoparticle vaccine.

2. The method of claim 1, wherein the TFF is performed at a feed flow rate of about 400 mL/minute and a permeate flow rate of about 100 mL/minute.

3. The method of claim 1, wherein the TFF is performed at a Feed pressure (Fp) of about 5.5, a Retentate pressure (Rp) of about 3.5, a Permeate pressure (Pp) of about 2.0 and a Transmembrane pressure (TMP) of about 2.5.

4. The method of claim 1, wherein step ii) comprises the steps of
(a) adding 200 proof dehydrated alcohol at 42.85 mL per 100 mL of concentrated bacterial lysate to a final concentration of 30% ethanol and stirring the mixture for about 2.5 hours at room temperature;
(b) incubating the mixture produced in step (a) overnight at room temperature to allow a precipitate and a clear ethanol-lysate phase to form;
(c) separating the clear ethanol-lysate phase from the precipitate; and
(d) adjusting the ethanol concentration of the ethanol-lysate phase to 25%.

5. The method of claim 1, wherein step ii) reduces a level of endotoxin in the concentrated bacterial lysate.

6. The method of claim 1, wherein step iii) comprises concentrating the ethanol-treated bacterial lysate to about 50 mL.

7. The method of claim 1, wherein step iv) comprises using a UV water purifier system with UV monitor to treat the ethanol-treated bacterial lysate.

8. The method of claim 1, wherein step iv) inactivates lambda-phage in the ethanol-treated bacterial lysate.

9. The method of claim 1, wherein a level of endotoxin in the nanoparticle vaccine is below about 10 EU/10¹⁰ particles, below about 1.5 EU/10¹⁰ particles, below about 1.2 EU/10¹⁰ particles or below about 1.0 EU/10¹⁰ particles.

10. The method of claim 1, wherein the level of endotoxin in the nanoparticle vaccine is reduced about 25%, about 30%, about 35%, about 40%, about 45%, about 50%, about 75%, about 80%, about 90% or about 99% compared to the level of endotoxin in the bacterial lysate.

11. The method of claim 1, wherein the cancer antigen is expressed on human cancer cells.

12. The method of claim 1, wherein the cancer antigen is human aspartyl (asparaginyl) β-hydroxylase (HAAH).

13. The method of claim 1, wherein the lambda-phage expresses amino acids 113-311 from the N-terminal region of HAAH fused at the C-terminus of the lambda-phage head decoration protein D (gpD).

14. The method of claim 1, wherein the lambda-phage expresses or comprises a protein comprising the amino acid sequence of SEQ ID NO:5 fused at the C-terminus of the lambda-phage head decoration protein D (gpD).

15. The method of claim 1, wherein the lambda-phage expresses or comprises a protein comprising the amino acid sequence of SEQ ID NO:4.

16. The nanoparticle vaccine produced by the method of claim 1.

17. A method for eliciting an antibody response in a subject, the method comprising administering to the subject an effective amount of the nanoparticle vaccine of claim 16.

18. The method of claim 17, wherein the subject has prostate, liver, bile duct, brain, breast, colon, ovarian or pancreatic cancer or a hematological malignancy.

19. A method for treating a symptom of or ameliorating cancer in a subject, the method comprising administering to the subject an effective amount of the nanoparticle vaccine of claim 16.

20. The method of claim 17, wherein the cancer is head-and-neck, lung, prostate, liver, bile duct, brain, breast, colon, ovarian or pancreatic cancer or a hematological malignancy.

21. The method of claim 18, wherein the subject has a biochemical recurrence of prostate cancer.

22. The method of claim 18, wherein the hematological malignancy is chronic myelomonocytic leukemia or myelodysplastic syndrome.

23. The method of claim 18, wherein the cancer is HAAH-expressing cancer.

24. The method of claim 17, wherein the nanoparticle vaccine is administered at a dose from about 2×10¹⁰ particles up to about 3×10¹¹ particles.

25. The method of claim 17, wherein the nanoparticle vaccine is administered at a dose of about 1×10¹¹ particles.

26. The method of claim 17, wherein up to 15 cycles of the nanoparticle vaccine are administered, and wherein each cycle comprises a treatment period and a rest period.

27. The method of claim 26, wherein the treatment period is about 1 day, and the rest period is about 20 days.

28. The method of claim 26, wherein the treatment period is about 1 day, and the rest period is about 41 days.

29. The method of claim 26, wherein the treatment period is about 1 day, and the rest period is about 71 days.

30. The method of claim 26, wherein four cycles are administered.

31. The method of claim 26, wherein six cycles are administered.

32. The method of claim 25, wherein a dose of about 1×10¹¹ particles is administered every 3 weeks until week 12; and then a dose of about 1×10¹¹ particles is administered every 6 weeks until week 45.

33. The method of claim 26, wherein the nanoparticle vaccine is administered until the subject exhibits disease progression or toxicity.

34. The method of claim 29, wherein the nanoparticle vaccine is administered for up to 24 months if the subject does not exhibit disease progression.

35. A method for eliciting an antibody response in a subject, the method comprising administering to the subject an effective amount of a nanoparticle vaccine comprising lambda-phage expressing or comprising a protein comprising the amino acid sequence of SEQ ID NO:4, wherein the nanoparticle vaccine is administered at a dose from about 2×10¹⁰ particles up to about 3×10¹¹ particles.

36. The method of claim 35, wherein the subject has head-and-neck, lung, prostate, liver, bile duct, brain, breast, colon, ovarian or pancreatic cancer or a hematological malignancy.

37. A method for treating a symptom of or ameliorating cancer in a subject, the method comprising administering to the subject an effective amount of a nanoparticle vaccine comprising lambda-phage expressing or comprising a protein comprising the amino acid sequence of SEQ ID NO:4, wherein the nanoparticle vaccine is administered at a dose from about 2×10¹⁰ particles up to about 3×10¹¹ particles.

38. The method of claim 37, wherein the cancer is head-and-neck, lung, prostate, liver, bile duct, brain, breast, colon, ovarian or pancreatic cancer or a hematological malignancy.

39. The method of claim 36, wherein the subject has a biochemical recurrence of prostate cancer.

40. The method of claim 36, wherein the hematological malignancy is chronic myelomonocytic leukemia or myelodysplastic syndrome.

41. The method of claim 36, wherein the cancer is HAAH-expressing cancer.

42. The method of claim 35, wherein the nanoparticle vaccine is administered at a dose of about 2×10¹⁰ particles, about 1×10¹¹ particles or about 3×10¹¹ particles.

43. The method of claim 35, wherein up to 15 cycles of the nanoparticle vaccine are administered, and wherein each cycle comprises a treatment period and a rest period.

44. The method of claim 43, wherein the treatment period is about 1 day, and the rest period is about 20 days.

45. The method of claim 43, wherein the treatment period is about 1 day, and the rest period is about 41 days.

46. The method of claim 43, wherein the treatment period is about 1 day, and the rest period is about 71 days.

47. The method of claim 35, wherein four cycles are administered.

48. The method of claim 35, wherein six cycles are administered.

49. The method of claim 42, wherein a dose of about 1×10¹¹ particles is administered every 3 weeks until week 12; and then a dose of about 1×10¹¹ particles is administered every 6 weeks until week 45.

50. The method of claim 35, wherein the nanoparticle vaccine is administered until the subject exhibits disease progression or toxicity.

51. The method of claim 46, wherein the nanoparticle vaccine is administered for up to 24 months if the subject does not exhibit disease progression.
